(19) United States
(12) Patent Application Publication
(10) Pub. No.: US 2026/0366010 A1
(43) Pub. Date: Nov. 19, 2026

(54) RETRIEVAL OF SEQUENCE-VERIFIED NUCLEIC ACID MOLECULES

(71) Applicant: Thermo Fisher Scientific GENEART GmbH, Regensburg (DE)

(72) Inventor: Phillip Kuhn, Regensburg (DE)

(21) Appl. No.: 19/137,930

(22) PCT Filed: Dec. 19, 2022
(86) PCT No.: PCT/EP2022/086706

§ 371 Date: Jun. 11, 2025

Related U.S. Application Data
(60) Provisional application No. 63/291,738, filed on Dec. 20, 2021.

(51) Int. Cl. C12N 15/10

(52) U.S. Cl. CPC C12N 15/1065

(57) ABSTRACT

The present disclosure generally relates to compositions and methods for the identification of desired nucleic acid molecules in one or more mixtures of nucleic acid molecules. Provided, as examples, are compositions and methods for high throughput synthesis, assembly and isolation of nucleic acid molecules in many instances, with high sequence fidelity. The disclosed compositions and methods allow, in part, for efficient production and selection of synthetic nucleic acids of any size without cell-based cloning workflows.

### FIELD OF THE INVENTION

The present disclosure generally relates to compositions and methods for the identification of desired nucleic acid molecules in one or more mixtures of nucleic acid molecules. Provided, as examples, are compositions and methods for high throughput synthesis, assembly and isolation of nucleic acid molecules in many instances, with high sequence fidelity. The disclosed compositions and methods allow, in part, for efficient production and selection of synthetic nucleic acids of any size without cell-based cloning workflows.

### BACKGROUND OF THE INVENTION

Over the years gene synthesis has become more cost effective and efforts to develop high throughput synthesis platforms and miniaturize certain workflows offers new applications and market opportunities.

With progress in genetic engineering, a need for the generation of larger nucleic acid molecules has developed. In many instances, nucleic acid assembly methods start with the synthesis of relatively short nucleic acid molecules (e.g., chemically synthesized oligonucleotides), followed by the generation of double-stranded fragments or sub-assemblies (e.g., by annealing and elongating multiple overlapping oligonucleotides), and often proceeds to build larger assemblies such as genes, operons or even functional biological pathways (e.g., by ligation, enzymatic elongation, recombination or a combination thereof). Where nucleic acid molecules are assembled from chemically synthesized oligonucleotides, such assembled molecules often have errors. The probability of a synthetic nucleic acid sequence being error-free decreases exponentially as its length increases. Thus, a large number of assembly products typically need to be sequenced to obtain one error-free molecule.

One of the bottlenecks in synthetic biology and nucleic acid synthesis is the requirement of cells to produce single clones comprising desired nucleic acid molecules, which can be sequence-verified and further processed. While standard cloning procedures based on cells are useful to select for nucleic acid molecules that have been properly transferred into a vector of choice by using antibiotic resistance markers, single cell clones are also used to screen, identify and select those clones harboring a desired nucleic acid molecule from other clones carrying undesired molecules with erroneous sequences. Because of the time to grow cell colonies (e.g. from transformed bacteria) and the difficulties of automating the process of transforming and growing cells, picking single colonies and analyzing the obtained clones, there is a desire for simplified processes that rely exclusively on molecular biology techniques.

Different approaches to address this problem have been described. The first technique which is generally known as a “dial-out” PCR is based on the tagging of single molecules in a mixture of diverse nucleic acid assembly products with barcodes, sequencing the tagged molecules to identify error-free sequences, and subsequent retrieval of the desired molecules by PCR amplification with barcode-specific primers (Schwartz et al., (2012) Accurate gene synthesis with tag-directed retrieval of sequence-verified DNA molecules. Nat. Methods, 9, 913-915). Although this method allows for efficient retrieval of target nucleic acid molecules from a large pool, it has a couple of disadvantages, namely: (1) the length of nucleic acid fragments that can be analyzed is limited to the read-length of the used next generation sequencing platform; (2) even if a high fidelity polymerase is used, it cannot be completely excluded that new errors will be introduced during the PCR-based retrieval step, (3) a large set of barcode primers is needed to specifically amplify desired molecules from a mixed pool of high complexity, and (4) the retrieved final molecule is not sequence-verified. An alternative method is “digital PCR” (also referred to as single molecule PCR or limiting dilution PCR), where a sample of mixed nucleic acid molecules is partitioned to the level of single molecules (e.g. in wells of a microwell plate). PCR amplification is then performed in all compartments and an all-or-none (i.e. digital) signal is obtained which can be used to either determine the sequence of a target molecule or calculate the number of target molecules by using the Poisson distribution (see e.g. Morley A. “Digital PCR: A brief history” Biomol Detect. Quantif. 2014 1 (1): 1-2). This method, however, requires a multitude of PCR reactions to be performed in well-plate formats to allow for the tracing back of sequence-verified molecules to respective well locations and is therefore tedious and wasteful.

Thus, there is a need for a cell-free cloning and sequence verification process that is agnostic (i.e. compatible with different sequencing platforms) and allows for high quality results at limited investment of time and materials.

The present disclosure addresses this problem by providing an in vitro cloning method (also referred to as “UMI cloning”) that is free of cells and can produce de novo synthesized linear nucleic acid molecules of any size at a high correctness.

### SUMMARY OF THE INVENTION

The present invention in at least some embodiments, overcomes the drawbacks of the background art by providing compositions and methods for producing, identifying and retrieving sequence-correct nucleic acid molecules from complex nucleic acid mixtures. The invention is set out in the appended claims.

In particular, in one embodiment the invention provides a method for identifying a nucleic acid molecule having a desired sequence in a mixture of nucleic acid molecules comprising the following steps: (a) providing one or more mixtures of nucleic acid molecules, each mixture comprising a plurality of nucleic acid molecules designed to have a desired sequence, wherein each nucleic acid molecule optionally comprises a linker at the 5′ end and at the 3′ end; (b) providing a set of nucleic acid tags, each tag comprising at least (i) a barcode, and (ii) a handle or adapter at the 5′ end; (c) optionally determining the concentration of nucleic acid molecules in each of the one or more mixtures; (d) providing one or more first compartments and diluting each of the one or more mixtures of nucleic acid molecules in a separate first compartment to obtain diluted mixtures of nucleic acid molecules, each diluted mixture comprising a predetermined amount of nucleic acid molecules; (e) contacting the diluted mixtures of nucleic acid molecules in one or more of the first compartments with the set of nucleic acid tags and attaching a tag to both ends of substantially each nucleic acid molecule in the one or more first compartments to obtain tagged nucleic acid molecules having a barcode region and a handle or adapter region at both ends; (f) optionally providing one or more pairs of amplification primers designed to hybridize to both ends of the tagged nucleic acid molecules and amplifying the tagged nucleic acid molecules in the one or more first compartments; (g) optionally purifying the amplified nucleic acid molecules; (h) providing a set of barcode primers, each barcode primer of the set comprising a barcode-specific region designed to hybridize to a specific barcode in the set of nucleic acid tags; (i) providing one or more second compartments and contacting at least a portion of each of the optionally amplified and optionally purified tagged nucleic acid molecules with a defined pair of the set of barcode primers in a second compartment and performing an amplification reaction in each second compartment to obtain one or more second compartments comprising: (i) one specific amplification product, (ii) no specific amplification product, and/or (iii) two or more specific amplification products; (j) optionally identifying second compartments (ii) having no specific amplification product and/or second compartments (iii) having two or more specific amplification products and excluding such second compartments from subsequent steps (k) optionally pooling at least a portion of the amplification products from two or more of the second compartments; (1) sequencing the amplification products to obtain sequence data; (m) analysing the sequence data, and (n) identifying one or more second compartments comprising one specific amplification product, thereby identifying one or more nucleic acid molecules having the desired sequence. Specific embodiments and alternatives of method steps are set out in the dependent claims and are further described below.

In an alternative embodiment the invention provides a method for identifying a nucleic acid molecule having a desired sequence in a mixture of nucleic acid molecules comprising the following steps: (a) providing one or more mixtures of nucleic acid molecules, each mixture comprising a plurality of nucleic acid molecules designed to have a desired sequence, wherein each nucleic acid molecule optionally comprises a linker at the 5′ end and at the 3′ end; (b) providing a set of nucleic acid tags, each tag comprising at least (i) a barcode, and (ii) a handle or adapter at the 5′ end; (c) contacting the one or more mixtures of nucleic acid molecules with the set of nucleic acid tags and attaching a tag to both ends of substantially each nucleic acid molecule to obtain tagged nucleic acid molecules having a barcode region and a handle or adapter region at both ends; (d) optionally determining the concentration of tagged nucleic acid molecules in one or more mixtures; (e) providing one or more first compartments and diluting each of the one or more mixtures of tagged nucleic acid molecules in a separate first compartment to obtain diluted mixtures of tagged nucleic acid molecules, each diluted mixture comprising a predetermined amount of tagged nucleic acid molecules; (f) optionally providing one or more pairs of amplification primers designed to hybridize to both ends of the tagged nucleic acid molecules and amplifying the tagged nucleic acid molecules in the one or more first compartments; (g) optionally purifying the amplified nucleic acid molecules; (h) providing a set of barcode primers, each barcode primer of the set comprising a barcode-specific region designed to hybridize to a specific barcode in the set of nucleic acid tags; (i) providing one or more second compartments and contacting at least a portion of each of the optionally amplified and optionally purified tagged nucleic acid molecules with a defined pair of the set of barcode primers in a second compartment and performing an amplification reaction in each second compartment to obtain one or more second compartments comprising: (i) one specific amplification product, (ii) no specific amplification product, and/or (iii) two or more specific amplification products; (j) optionally identifying second compartments (ii) having no specific amplification product and/or second compartments (iii) having two or more specific amplification products and excluding such second compartments from subsequent steps; (k) optionally pooling at least a portion of the amplification products from two or more of the second compartments; (1) sequencing the amplification products to obtain sequence data; (m) analysing the sequence data, and (n) identifying one or more second compartments comprising one specific amplification product, thereby identifying one or more nucleic acid molecules having the desired sequence.

Further embodiments are outlined in the disclosure and dependent claims.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative examples, in which the principles of the disclosure are utilized, and the accompanying drawings of which:

FIG. 1 is a schematic of a workflow of various aspects of the disclosure.

FIG. 2 is a schematic of a PCR-based process for assembling nucleic acid molecules. (a) Overlapping forward and reverse oligonucleotides are extended in the first cycle round. (b) Extended assembly products anneal to each other and are further extended in the second cycle round. (c) Further extensions take place in subsequent rounds and full-length product accumulates. Two terminal primers (1) and (2) can also be universal and applied in excess within the PCR mix to allow a controlled amplification of assembly products.

FIG. 3 is a schematic representation of multiplex nucleic acid assembly workflows. (A) shows beads in a vessel, where oligonucleotides on these beads are released into solution and are then used for assembly of a single nucleic acid fragment. (B) shows beads in a vessel, where oligonucleotides from these beads are components of more than one assembly product (e.g., multiple fragments). These oligonucleotides released into a single pool for multiplex assembly (e.g., by PCR) are presented directly under (B).

FIG. 4 is a comparison of a “dial-out” workflow (A) and a workflow according to various aspects of the disclosure (B). In a “dial-out” procedure a mixture of nucleic acid molecules having different sequences is tagged with an abundant set of barcodes and the barcoded nucleic acid molecules are then diluted and amplified into “families”. The pool of amplified nucleic acid molecules is then subject to next generation sequencing (“NGS”) to identify molecules that do not have errors and determine the barcodes flanking those error-free molecules. Primers with sequences complementary to the respective barcodes flanking the error-free molecules are then synthesized and used to specifically amplify (i.e., dial-out) the desired target molecules. In contrast, in the workflow illustrated in FIG. 4B a predetermined number of barcoded target molecules are optionally amplified in separate compartments (e.g., wells of a microwell plate) with known barcode combination-specific primer pairs before they are sequenced. After identification of an error-free target molecule by sequencing all molecules comprising the correct sequence may be directly retrieved from the respective compartment based on the known position (e.g., a well within a multi-well plate) of the barcode combination-specific primer pairs without further amplification thus avoiding incorporation of additional errors into a sequence-verified nucleic acid fragment. In some examples, molecule mixtures comprising single fragments may first be diluted to a predetermined number of molecules and then tagged with a limited number of barcodes, whereas in other examples, molecule mixtures comprising single fragments may first be tagged with a limited number of barcodes and then diluted to a predetermined number of molecules.

FIG. 5 is a schematic of an exemplary UMI cloning workflow. In step (1) one or more (e.g., between 10 and 1,000) mixtures of nucleic acid molecules some of which comprise errors (indicated by asterisks) are each diluted to a predetermined number of molecules; in step (2) tags comprising a known set of barcodes (having a predefined number of specific barcode sequences) and universal sequencing adapters (or universal handles for attaching sequencing adapters) are added to each mixture of diluted molecules, and the tags are attached (e.g., by PCR or ligation) to both ends of the nucleic acid molecules in step (3). Alternatively, in some embodiments the dilution in step (1) and the tagging in steps (2) and (3) may be conducted in reverse order. In step (4) the tagged molecules are amplified by PCR into “families” with all family members comprising the same barcode combination; in step (5) aliquots of the barcoded and amplified molecules are transferred to a multi-well plate or other suitable compartments and are combined with individual barcode-specific primer pairs of known sequence (e.g., wells of a 96-well plate may be pre-loaded with different primer pairs); in step (6) a “dial-out” PCR is conducted with the preloaded primers which will only result in an amplification product if molecules comprising the complementary barcode combination are present in the respective compartment. As indicated by the circle symbols, the dial-out PCR in step (6) may result in (i) “empty compartments” with no barcode-primer specific amplification product (empty/white circles); (ii) “single clones” with one specific amplification product resulting from one exact match of a barcode-specific primer pair (full black circles), or (iii) two or more “mixed clones” with two or more amplification products resulting from several matches of a barcode-specific primer pair (striped circles); in optional step (7) empty compartments not comprising barcode primer-specific amplification products (and optionally compartments having more than one specific amplification product) may be identified and removed from subsequent steps; in step (8) the samples are sequenced; in step (9) the sequencing results are analysed. Sequences derived from mixed clones will result in ambiguous data and be discarded. Single clones can be screened for correct ones (e.g., by matching against the desired target sequence) and the barcode sequence combination associated with the correct clones is used to locate the desired molecules in respective compartments based on the known position of the corresponding barcode primer pair. Identified molecules may then be selectively retrieved from identified compartments and used for downstream procedures.

FIG. 6 is another schematic of a proposed workflow for simultaneous processing of multiple nucleic acid mixtures. Concentrations of various mixtures of nucleic acid molecules 1-n may be measured (e.g., using UV/Vis such as NanoDrop), fluorescence such as qPCR or Qbit, electrophoresis etc.) and mixtures may be diluted to a predetermined number of molecules (e.g., 37,000 per compartment). Tags containing barcodes are then added and attached to the diluted mixtures of nucleic acid molecules by ligation or a limited number of PCR cycles. In alternative examples, mixtures of nucleic acid molecules may first be tagged and then diluted. Tagged molecules may then optionally be amplified into families and mixed with barcode primers (e.g., preloaded into a PCR plate). Separate PCRs are then conducted in separate compartments yielding either clonal populations, mixed clones or no amplification product as described in more detail below. Samples may then be sequenced using either Sanger sequencing or other platforms such as next generation sequencing, wherein NGS sequencing may comprise pooling of multiple samples. Following analysis of sequencing reads, sequence-verified PCR products can be identified based on barcode combinations and retrieved from respective wells.

FIG. 7 shows an exemplary Monte-Carlo simulation for dilution of nucleic acid molecules to obtain an optimal number of compartments with “single families” (i.e., having only one specific amplification product). A set of 192×192 barcodes (5′ and 3′, respectively) yielding 36,864 combinations was used in this example and nucleic acid molecules from 24 PCR wells were analyzed. (A) shows a setting with 36,864 molecules resulting in nearly balanced numbers of wells with “zero”, “one”, or “two or more” amplified clones (A); (B) shows a setting with 29,491 molecules (−20% of aimed concentration) resulting in a majority of wells with zero amplified clones, whereas (C) shows a setting with 44,236 molecules (+20% of aimed concentration) resulting in a majority of wells with two or more amplified clones.

FIG. 8 is a schematic of an exemplary tagging procedure according to methods disclosed herein. (A) shows a set of tags (tag 1 and tag 2) for attachment to the 5′ and 3′ ends of a target nucleic acid molecule, each tag comprising at least a 5′ handle (Handle 1, Handle 2) and a barcode (Barcode 1, Barcode 2), and an optional linker at the 3′ end. (B) shows a tagged nucleic acid molecule after PCR-based attachment of tag 1 and tag 2 through hybridization of the linkers to complementary sequence regions at both ends of the target nucleic acid molecule. (C) the tagged nucleic acid molecules may be amplified using terminal amplification primers (Amplification Primer 1, Amplification Primer 2).

FIG. 9 illustrates an exemplary workflow according to various examples disclosed herein. In this example, the concentration of nucleic acid fragments derived from PCR-based assembly of oligonucleotides is measured by UV/Vis (NanoDrop) and samples are diluted in wells of a 96-well plate to obtain approximately 37,000 molecules per well. Barcodes and sequencing adapters are attached to both ends by 2-cycle PCR. Alternatively, nucleic acid samples may first be tagged and then diluted after optionally having determined the concentration of the tagged nucleic acid fragments. Tagged fragments may then optionally be amplified into families by PCR. Aliquots of each amplified sample are transferred to separate wells of a 96-well plate and a different pair of barcode primers is added to each well and molecules comprising the corresponding barcode combinations are amplified. Amplification products may be verified by DNA quantification methods such as NanoDrop or gel electrophoresis. Samples from wells containing amplification products are then sequenced as described elsewhere herein (e.g., by Sanger sequencing, NGS or 3^{rd} generation sequencing platforms) and correct nucleic acid fragments are identified and traced back to the respective well based on their barcode combinations and the well comprising the barcode-complementary primer pair. Error-free nucleic acid fragments may then be retrieved and further processed or cloned as described elsewhere herein.

FIG. 10 shows data generated in the in vitro cloning workflow described in Example 2. Table (A) lists synthetic DNA fragments and concentrations used in the example; (B) shows an E-Gel image of the various pre-assembled fragments; (C) shows an E-Gel image of the diluted, tagged and amplified fragments; (D) shows an exemplary schematic of a 96-well plate, wherein each well is pre-loaded with a specific pair of 5′ and 3′ barcode primers; € and (F) show E-Gel images of the fragments amplified with barcode-specific primer pairs in wells of the 96-well plate; (G) depicts a chromatogram of a clonal sequence derived from an exemplary well of the 96-well plate and (H) depicts a chromatogram of a mixed sequence derived from a different well of the 96-well plate.

FIG. 11 depicts a UMI cloning workflow using 3rd generation long read sequencing (A). The UMI cloning workflow comprises the steps of diluting and tagging molecule mixtures (wherein such order may be reversed), amplification into families followed by dial-out PCR with specific barcode primers. Amplification products are then combined and subject to 3rd generation sequencing including UMI-based clustering and consensus sequence calculation to determine correct sequences. Barcode combinations of error-free molecules are then matched against amplification compartments containing corresponding primer pairs to retrieve sequence-correct molecules. In some instances, concatemers may be built (e.g., using Gibson cloning) from individual tagged clones to increase throughput on long read sequencing platforms (B).

FIG. 12 depicts an exemplary workflow for automation of high-throughput UMI-cloning. In step 1. one or more 1536 multi-well plates compatible with an acoustic liquid handler (e.g., Labcyte Echo®) are loaded with defined barcode primer combinations. In step 2. diluted and tagged mixtures of nucleic acid molecules are added to wells of the plate. In step 3. the plates are sealed, and dial-out PCR reactions are conducted in a Hydrocycler. In step 5. small volumes from each well are transferred to a next/third generation sequencing sample prep reaction. In step 6. sequencing is performed and in step 7. sequence verified clones are selected for subsequent processing steps.

FIG. 13 depicts an alternative UMI-cloning method using a quality control (“QC”) sequence. In step (a) single molecules in a mixture are tagged with tags comprising adapter and barcode sequences (BC1, BC2) as well as short unknown and random QC sequences (QC1, QC2). Fragments are provided with universal linkers (L1, L2) for PCR-based tagging. Following amplification into “families” using adapter primers in step (b) the QC-containing molecules are combined with different sets of barcode primers in multiple compartments. In step (c) a dial-out PCR reaction is performed.

FIG. 14 illustrates how quality control (“QC”) sequences can be used to distinguish “mixed clones” from “single clones”. (A) shows an exemplary mixed clone comprising an error-free (I) and an error containing (II) nucleic acid molecule both of which have obtained the same set of barcode sequences (BC1, BC2) but different sets of quality control (QC) sequences through universal linker-based PCR tagging (L1, L2). Handles may be present for downstream processing. (B) shows an exemplary single clone comprising two nucleic acid molecules ((I) and (II), respectively) having the same sets of BC and QC sequences. (C) describes an exemplary method for identifying mixed and single clones. Sequencing data of mixed nucleic acid molecules are processed by searching and grouping known barcodes. QC sequences are then extracted from the grouped sequences to calculate a consensus sequence. If a consensus of QC sequences does not match, the clone is identified as a mixed clone. If a consensus of QC sequences matches, the clone is identified as a (single) clone. Finally, the consensus of identified clone sequences can be further analyzed to obtain those that do not contain errors.

### DETAILED DESCRIPTION

### Definitions

The term “nucleic acid molecule” as used herein refers to a covalently linked sequence of nucleotides or bases (e.g., ribonucleotides for RNA and deoxyribonucleotides for DNA but also include DNA/RNA hybrids where the DNA is in separate strands or in the same strands) in which the 3′ position of the pentose of one nucleotide is joined by a phosphodiester linkage to the 5′ position of the pentose of the next nucleotide. Nucleic acid molecules may be single- or double-stranded or partially double-stranded. Nucleic acid molecules may appear in linear or circularized form in a supercoiled or relaxed formation with blunt or sticky ends and may contain “nicks”. Nucleic acid molecules may be composed of completely complementary single strands or of partially complementary single strands forming at least one mismatch of bases. Nucleic acid molecules may further comprise two self-complementary sequences that may form a double-stranded stem region, optionally separated at one end by a loop sequence. The two regions of nucleic acid molecules which comprise the double-stranded stem region are substantially complementary to each other, resulting in self-hybridization. However, the stem can include one or more mismatches, insertions or deletions.

Nucleic acid molecules may comprise chemically, enzymatically, or metabolically modified forms of nucleotides or combinations thereof. Chemically synthesized nucleic acid molecules may refer to nucleic acids typically less than or equal to 200 nucleotides long (e.g., between 5 and 200, between 10 and 150, between 15 and 100, or between 20 and 50 nucleotides in length), whereas enzymatically synthesized nucleic acid molecules may encompass smaller as well as larger nucleic acid molecules as described elsewhere herein. Enzymatic synthesis of nucleic acid molecules may include stepwise processes using enzymes such as polymerases, ligases, exonucleases, endonucleases, recombinases or the like or a combination thereof. Thus, the disclosure provides, in part, compositions and combined methods relating to the enzymatic assembly of chemically synthesized nucleic acid molecules.

A nucleic acid molecule has a “5′ terminus” and a “3′ terminus” because nucleic acid molecule phosphodiester linkages occur between the 5′ carbon and 3′ carbon of the pentose ring of the substituent mononucleotides. The end of a nucleic acid molecule at which a new linkage would be to a 5′ carbon is its 5′ terminal nucleotide. The end of a nucleic acid molecule at which a new linkage would be to a 3′ carbon is its 3′ terminal nucleotide. A terminal nucleotide or base, as used herein, is the nucleotide at the end position of the 3′- or 5′-terminus. A nucleic acid molecule region, even if internal to a larger nucleic acid molecule (e.g., a sequence region within a nucleic acid molecule), also can be said to have 5′ and 3′ ends. Nucleic acid molecule also refers to short nucleic acid molecules, often referred to as, for example, primers or probes. Also, the terms “5′” and “3′” refer to strands of nucleic acid molecules. Thus, a linear, single-stranded nucleic acid molecule will have a 5′ terminus and a 3′ terminus. However, a linear, double-stranded nucleic acid molecule will have a 5′ terminus and a 3′ terminus for each strand. Thus, for nucleic acid molecules that encode proteins, for example, the 3′ terminus of the sense strand may be referred to.

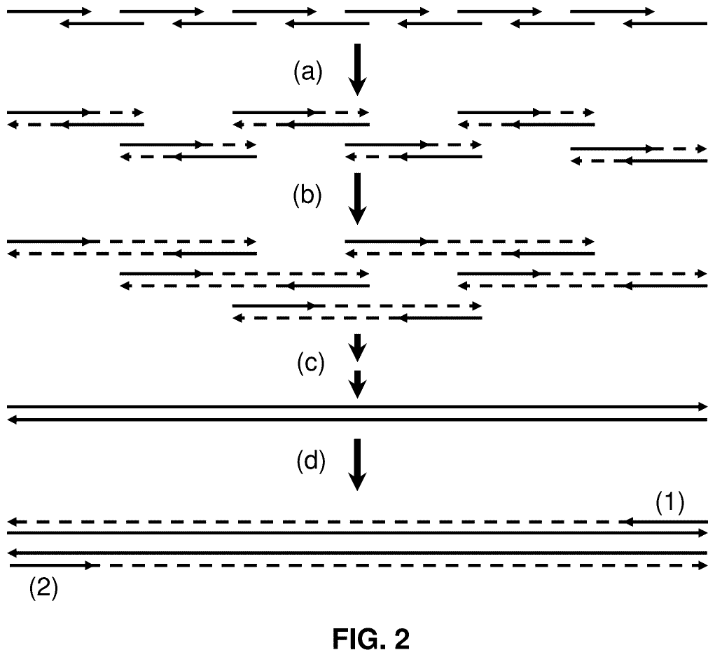

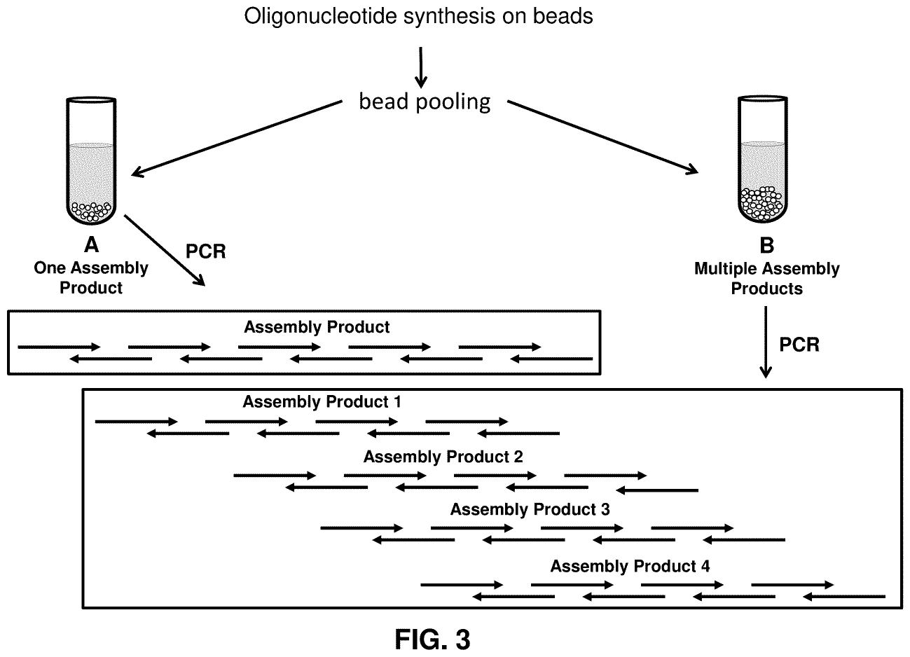

The term “oligonucleotide”, as used herein, refers to DNA and RNA, and to any other type of nucleic acid molecule that is an N-glycoside of a purine or pyrimidine base but will typically be DNA. Oligonucleotides are thus a subset of nucleic acid molecules and may be single-stranded or double-stranded. Oligonucleotides (including primers as described below) may be referred to as “forward” or “reverse” to indicate the direction in relation to a given nucleic acid sequence. For example, a forward oligonucleotide may represent a portion of a sequence of the first strand of a nucleic acid molecule (e.g., the “sense” strand), whereas a reverse oligonucleotide may represent a portion of a sequence of the second strand (e.g., “antisense” strand) of said nucleic acid molecule or vice versa. In many instances, a set of oligonucleotides used to assemble longer nucleic acid molecules will comprise both forward and reverse oligonucleotides capable of hybridizing to each other via complementary regions (as illustrated e.g., in FIGS. 2 and 3). Oligonucleotides are typically less than 200 nucleotides, more typically less than 100 nucleotides in length. Thus, “primers” will generally fall into the category of oligonucleotide. Oligonucleotides can be prepared by any suitable method, including direct chemical synthesis by a method such as the phosphotriester method of Narang et al., Meth. Enzymol. 68:90-99 (1979); the phosphodiester method of Brown et al., Meth. Enzymol. 68:109-151 (1979); the diethylphosphoramidite method of Beaucage et al., Tetrahedron Letters 22:1859-1862 (1981); and the solid support method of U.S. Pat. No. 4,458,066. A review of synthesis methods of conjugates of oligonucleotides and modified nucleotides is provided in Goodchild, Bioconjugate Chemistry 1:165-187 (1990). 1. Alternatively, oligonucleotides may be synthesized using enzymatic methods as described by Hoff et al. 2019 or Lee et al., Nat Commun 10 (1): 2383 (2019). An overview and comparison of available methods for large-scale oligonucleotide synthesis is provided in Song et al., Front. Bioeng. Biotechnol., 2021. Where appropriate, the term oligonucleotide may refer to a primer or probe and these terms may be exchangeably used herein.

Whereas probes may be typically used to detect at least partially complementary nucleic acid molecules, primers are often referred to as starter nucleic acid molecules for enzymatic assembly or extension reactions. Thus, the terms “primer”, “amplification primer”, “sequencing primer”, “barcode primer” and the like as used herein, refers to a short nucleic acid molecule capable of acting as a point of initiation of nucleic acid synthesis under suitable conditions. Such conditions include those in which synthesis of a primer extension product complementary to a nucleic acid strand is induced in the presence of different nucleoside triphosphates (e.g., A, C, G, T and/or U) and an agent for extension (for example, a DNA polymerase or reverse transcriptase) in an appropriate buffer and at a suitable temperature. A primer is generally composed of single-stranded DNA but can be provided as a double-stranded or partially double-stranded molecule for specific applications (e.g., blunt end ligation). Optionally, a primer can be naturally occurring or synthesized using chemical synthesis of recombinant procedures. The appropriate length of a primer depends on the intended use of the primer but typically ranges from about 6 to about 200 nucleotides, including intermediate ranges, such as from about 10 to about 50 nucleotides, from about 15 to about 35 nucleotides, from about 18 to about 75 nucleotides and from about 25 to about 150 nucleotides. The design of suitable primers for the amplification of a given target sequence is well known in the art and described in the literature (see for example OLIGOPERFECT™ Designer, Thermo Fisher Scientific). Primers can incorporate additional features which allow for the detection or immobilization of the primer but do not alter the basic property of the primer, that of acting as a point of initiation of DNA synthesis.

In some examples, a primer includes a detectable moiety or label. The label can generate, or cause to generate, a detectable signal. In some examples, the detectable signal can be generated from a chemical or physical change (e.g., heat, light, electrical, pH, salt concentration, enzymatic activity, or proximity events). In some examples, the label can include compounds that are luminescent, photoluminescent, electroluminescent, bioluminescent, chemiluminescent, fluorescent, phosphorescent or electrochemical. In some examples, the label can include compounds that are fluorophores, chromophores, radioisotopes, haptens, affinity tags, atoms or enzymes. In some examples, the label comprises a moiety not typically present in naturally occurring nucleotides. For example, the label can include fluorescent, luminescent or radioactive moieties. Primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)n sequence, where n is in the range from about 20 to about 200). A primer can include an extendible 3′ end or a non-extendible 3′ end, where the terminal nucleotide at the non-extendible end carries a blocking moiety at the 2′ or 3′ sugar position. The region of the primer that is sufficiently complementary to the template or target nucleic acid molecule to hybridize is typically located in the 3′ region of a primer and referred to herein as the hybridizing or complementary region, or target-specific region. The primer can also include a region that is designed to exhibit minimal hybridization to a portion of the template nucleic acid molecule (e.g., a non-target specific sequence in the 5′ region of the primer). For example, the primer can include at least one tag in the 5′ non-target specific region.

A “universal” sequence as used herein refers to a region of sequence that is common to two or more nucleic acid molecules, e.g., a primer binding site that is present in a plurality of nucleic acid molecules, where the molecules also have regions of sequence that differ from each other. A universal sequence that is present in different members of a collection of molecules can allow capture of multiple different nucleic acids using a population of universal capture nucleic acids that are complementary to a portion of the universal sequence, e.g., a universal primer binding site. Non-limiting examples of universal primer binding sites include sequences that are identical to or complementary to P5 and P7 primers. Similarly, a universal sequence present in different members of a collection of molecules can allow the replication or amplification of multiple different nucleic acids using a population of universal primers that are complementary to a portion of the universal sequence, e.g., a universal primer binding site. Thus, a universal primer includes a sequence that can hybridize specifically to a universal sequence. In some context herein, universal primer binding sites may be included in universal “linkers” as described in more detail below. Target nucleic acid molecules may be modified to attach universal adapters (as defined below), for example, at one or both ends of the different target sequences, as described herein. In another embodiment, a universal sequence that is present in different members of a collection of molecules can allow for removal of a portion of the universal adapter from the target molecule to which it is attached, e.g., a universal removal sequence, cleavage site etc.

A “sequencing primer”, as used herein refers to a primer that is used to initiate a sequencing reaction performed on a target nucleic acid molecule. The term “sequencing primer” refers to both a forward sequencing primer and to a reverse sequencing primer. typically comprises one or more adapters. In some examples, a sequencing primer may at least comprise a target-specific region, a barcode and/or an adapter. In some examples, a sequencing primer may comprise at least one universal sequence which includes an amplification primer sequence, one or more cleavable sites and/or a sequence for grafting to a support. In some examples a sequencing primer or segment thereof may be nuclease-resistant and may comprise at least one inter-nucleotide phosphorothioate bond that is not susceptible to nuclease cleavage. In some examples, a pair of sequencing primers may comprise a first sequencing primer comprising at least a target-specific region, a barcode and an adapter and a second sequencing primer comprising at least a target-specific region and an adapter.

A set of primers or tags used in the same amplification reaction may have melting temperatures that are substantially the same, where the melting temperatures are within about 10-5 °C of each other, or within about 5-2 °C of each other, or within about 2-0.5 °C of each other, or less than about 0.5 °C of each other.

In vitro cloning workflows described herein allow for identifying and retrieving sequence-correct nucleic acid molecules from a mixture of molecules some of which have errors based on the concept of “unique molecular indices” (UMIs). UMIs as used herein are sequences of nucleotides applied to or identified in nucleic acid molecules that may be used to distinguish individual molecules from one another. Since UMIs are used to identify nucleic acid molecules, they are also referred to as “unique molecular identifiers” (see, e.g., Kivioja, Nature Methods 9, 72-74 (2012)). In many instances UMI may be interchangeably used with “barcodes” and may have the same composition or length as described for barcodes herein. UMIs or barcodes may be sequenced along with the nucleic acid molecules with which they are associated to determine whether the read sequences are those of one source molecule or another.

Depending on the context in which they are used, the terms “UMI”, “tag”, “adapter”, “barcode”, “linker”, “handle” “QC” etc., may refer to both, the sequence information of a respective nucleic acid element and the physical molecule comprising such sequence element.

The terms “complementary” or “complementarity”, as used herein, refer to the natural binding of nucleic acid molecules (primers, oligonucleotides or polynucleotides etc.) under permissive salt and temperature conditions by base pairing. For example, the sequence “A-G-T” binds to the complementary sequence “T-C-A.” Complementarity between two single-stranded molecules may be “partial,” such that only some of the nucleic acids bind, or it may be “complete,” such that total complementarity exists between the single-stranded molecules. The degree of complementarity between nucleic acid strands has significant effects on the efficiency and strength of the hybridization between the nucleic acid strands. This is of particular importance in amplification reactions, which depend upon binding between nucleic acids strands. Complementary regions between nucleic acid molecules such as oligonucleotides may also be referred to as “overlaps” or “overlapping” regions as defined below.

As used herein, the term “consensus sequence” is a sequence determined after alignment of sequence reads associated with an input nucleic acid molecule generated from a sequencer by determining the base which is the most commonly found at each position in the compared, aligned sequence reads.

The term “hybridization”, as used herein, refers to any process by which a strand of nucleic acid binds with a complementary strand through base pairing. Hybridization and the strength of hybridization (for example, the strength of the association between the nucleic acids) is impacted by such factors as the degree of complementary between the nucleic acids, stringency of the conditions involved, the Tₘ of the formed hybrid, and the G:C ratio within the nucleic acids.

The term “homologous”, as used herein, refers to a degree of complementarity. Nucleic acid sequences may be partially or completely homologous (identical). A partially complementary sequence is one that at least partially inhibits a completely complementary sequence from hybridizing to a target nucleic acid and is referred to using the functional term “substantially homologous”.

The term “overlap” or “overlapping”, as used herein refers to a sequence homology or sequence identity shared by a portion of two or more oligonucleotides.

The term “gene” or “gene sequence”, as used herein generally refers to a nucleic acid sequence that encodes a discrete cellular product. In many instances, a gene or gene sequence includes a DNA sequence that comprises an open reading frame (ORF) and can be transcribed into mRNA which can be translated into polypeptide chains, transcribed into rRNA or tRNA or serve as recognition sites for enzymes and other proteins involved in DNA replication, transcription and regulation. These genes include, but are not limited to, structural genes, immunity genes, regulatory genes and secretory (transport) genes etc. However, as used herein, “gene” refers not only to the nucleotide sequence encoding a specific protein, but also to any adjacent 5′ and 3′ non-coding nucleotide sequence involved in the regulation of expression of the protein encoded by the gene of interest. These non-coding sequences include terminator sequences, promoter sequences, upstream activator sequences, regulatory protein binding sequences, and the like. In many instances, a gene is assembled from shorter oligonucleotides or nucleic acid fragments.

The terms “fragment”, “sub-fragment”, “segment”, or “component” or “element” or similar terms as used herein in connection with a nucleic acid molecule or sequence either refer to a product or intermediate product obtained from one or more process steps (e.g., synthesis, assembly, amplification etc.), or refer to a portion, part or template of a longer or modified nucleic acid product to be obtained by one or more process steps (e.g., assembly, amplification, construction, cloning etc.). In some instances, a nucleic acid fragment or sub-fragment may represent both, an assembly product (e.g., assembled from multiple oligonucleotides) and a starting compound for higher order assembly (e.g., a gene assembled from multiple fragments, or a fragment assembled from multiple sub-fragments etc.).

The term “full-length” as used herein in connection with a nucleic acid molecule or sequence refers to the “desired” length of a product nucleic acid molecule or sequence to be obtained by a given process step. The term full-length is therefore not limited to the length of the final or last nucleic acid product generated in a workflow or by a series of method steps, but refers, in some instances, to the desired length of an intermediate product of a particular process step. For example, a full-length oligonucleotide may be one that does not have truncations as a result of chemical synthesis.

As used herein, the term “sample” generally refers to anything capable of being analyzed by the methods provided herein that contains one or more target nucleic acid molecules or any fragments thereof. In an example, a sample may refer to a mixture of nucleic acid molecules having different sequences. For example, two separate mixtures of nucleic acid molecules comprising assembled nucleic acid fragments may be referred to as two different samples.

The term “vector”, as used herein refers to any nucleic acid molecule capable of transferring genetic material into a host organism. The vector may be linear or circular in topology and includes but is not limited to plasmids, viruses, bacteriophages. The vector may include amplification genes, enhancers or selection markers and may or may not be integrated into the genome of the host organism.

The term “plasmid”, as used herein refers to a vector that is able to be genetically modified to insert one or more nucleic acid molecules (e.g., assembly products). Plasmids will typically contain one or more region that renders it capable of replication in at least one cell type.

The term “amplification”, as used herein, relates to the production of additional copies of a nucleic acid molecule. Amplification as used herein is often carried out using polymerase chain reaction (PCR) technologies well known in the art (see, e.g., Dieffenbach, C. W. and G. S. Dveksler (1995) PCR Primer, a Laboratory Manual, Cold Spring Harbor Press, Plainview, N.Y.) but may also be carried out by other means including isothermal amplification methods such as, e.g., transcription mediated amplification, strand displacement amplification, rolling circle amplification, loop-mediated isothermal amplification, helicase-dependent amplification, single primer isothermal amplification or recombinase polymerase amplification (see, e.g., Fakruddin et al., “Nucleic acid amplification: Alternative methods of polymerase chain reaction”, J. Pharm Bioallied Sci, 2013, v. 5 (4), 245-252; or Gill and Ghaemi, “Nucleic acid isothermal amplification technologies: a review”, Nucleosides Nucleotides Nucleic Acids. 2008 27 (3), 224-43).

The terms “adjacent” or “flanking”, as used herein, refer to a position in a nucleic acid molecule immediately 5′ or 3′ to a reference region.

The term “transition”, when used in reference to the nucleotide sequence of a nucleic acid molecule, refers to a point mutation that changes a purine nucleotide to another purine (A↔G) or a pyrimidine nucleotide to another pyrimidine (C↔T).

The term “transversion”, when used in reference to the nucleotide sequence of a nucleic acid molecule, refers to a point mutation involving the substitution of a (two ring) purine for a (one ring) pyrimidine or a (one ring) pyrimidine for a (two ring) purine.

The term “indel”, as used herein, refers to the insertion or deletion of one or more bases in a nucleic acid molecule.

The term “sequence fidelity”, as used herein refers to the level of sequence identity of a nucleic acid molecule as compared to a reference sequence. Full identity being 100% identical over the full-length of the nucleic acid molecules being scored for sequence identity. Sequence fidelity can be measured in a number of ways, for example, by the comparison of the actual nucleotide sequence of a nucleic acid molecule to a desired nucleotide sequence (e.g., a nucleotide sequence that one wishes to be used to generate a nucleic acid molecule). Another way sequence fidelity can be measured is by comparison of sequences of two nucleic acid molecules in a reaction mixture. In many instances, the difference on a per base basis will be, on average, the same.

The term “error correction”, as used herein refers to changes in the nucleotide sequence of a nucleic acid molecule to alter a defect. These defects can be mismatches, insertions, deletions and/or substitutions. Defects can occur when a nucleic acid molecule that is being generated (e.g., by chemical or enzymatic synthesis) is intended to contain a particular base at a location but a different base is present at that location. Depending on context, error correction may either relate to the physical change (i.e., insertion, deletion or replacement) of one or more nucleotides or base pairs in a nucleic acid molecule or may relate to the correction of an in silico nucleic acid sequence (e.g., using software to correct errors resulting from nucleic acid sequencing).

The term “transformation”, as used herein, describes a process by which an exogenous nucleic acid molecule enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted nucleic acid is capable of replication either as an autonomously replicating plasmid or as part of the host chromosome. They also include cells that transiently express the inserted DNA or RNA for limited periods of time.

The terms “microchip”, “chip”, “synthesis chip”, or similar variations thereof as used herein will refer to an electronic computer chip on which oligonucleotide synthesis can occur.

The term “microarray” or “array”, as used herein refers to an array of distinct polynucleotides or oligonucleotides arrayed on a substrate, such as paper, nylon or any other type of membrane, filter, chip, glass slide, or any other suitable solid support. In certain instances, a microchip may represent a specific example of a microarray.

The terms “multiwell plate”, “microplate”, “microwell plate”, “plate” or similar variations thereof refers to a two-dimensional array of multiple wells located on a substantially flat surface. Multiwell plates can comprise any number of wells of any width or depth. In certain instances, a multiwell plate may be configured as a microchip. For example, when a material with well-like structures is overlaid onto a microchip.

The term “solid support”, as used herein refers to a porous or non-porous material on which polymers such as oligonucleotides or nucleic acid molecules can be synthesized and/or immobilized. As used herein “porous” means that the material contains pores which may be of non-uniform or uniform diameters (for example in the nm range). Porous materials include paper, synthetic filters, etc. In such porous materials, the reaction may take place within the pores. The solid support can have any one of a number of shapes, such as pin, strip, plate, disk, rod, fiber, bends, cylindrical structure, planar surface, concave or convex surface or a capillary or column. The solid support can be a particle, including bead, microparticles, nanoparticles and the like. The solid support can be a non-bead type particle (e.g., a filament) of similar size. The support can have variable widths and sizes. For example, sizes of a bead (e.g., a magnetic bead) which may be used in the practice of aspects of the disclosure may vary widely but include beads with diameters between 0.1 μm and 100 μm, between 1.0 μm and 50 μm, between 10 μm and 100 μm.

The support can be hydrophobic or capable of binding a molecule via hydrophobic interaction. The support can be hydrophilic or capable of being rendered hydrophilic and includes inorganic powders such as silica, magnesium sulfate, and alumina; natural polymeric materials, particularly cellulosic materials and materials derived from cellulose, such as fiber containing papers such as filter paper, chromatographic paper or the like. The support can be immobilized or located at an addressable position of a carrier such as, e.g., a multiwell plate or a microchip. The support can be loose or particulate (such as, e.g., a resin material or a bead in a well) or can be reversibly immobilized or linked to the carrier (e.g., by cleavable chemical bonds or magnetic forces etc.). For example, a plurality of nucleic acids having a desired sequence (such as a specific type of oligonucleotide) may be synthesized or provided on individual supports in an array device or carrier where each support is located at a uniquely addressable position such that one or more individual supports and specific nucleic acids bound thereto can be selectively retrieved from defined positions of the array or carrier.

As used herein an “addressable position” generally refers to a location within an array or carrier or compartment that may be readily identified so that a nucleic acid molecule located at a specific position may be individually processed at or retrieved from the one or more specific position of said array or carrier or compartment. In some instances, an array or carrier or compartment may be a chip or multiwell plate where each well is addressable or traceable based on its content (e.g., comprising a pre-defined set of molecules, primers, labeled moieties, barcodes etc.).

As used herein, the term “substantially” refers to the complete or nearly complete extent or degree of an action, characteristic, property, state, structure, item, or result. For example, attaching a tag to “substantially” each nucleic acid molecule in a mixture of nucleic acid molecules would mean that all or nearly all nucleic acid molecules in the mixture receive a tag. The exact allowable degree of deviation from absolute completeness may in some cases depend on the specific context. However, generally speaking the nearness of completion will be so as to have the same overall result as if absolute and total completion were obtained.

“a”, “an”, and “the” include plural reference unless the context clearly dictates otherwise. Thus, for example, reference to “an amplification primer” includes a single amplification primer as well as a plurality of amplification primers.

“one or more” as used herein with reference to mixtures of nucleic acid molecules, first or second compartments, pairs of amplification primers, etc. includes “two or more”, “three or more”, “five or more”, “ten or more” etc. or “at least two”, “at least three”, at least five”, “at least ten” etc.

### Overview

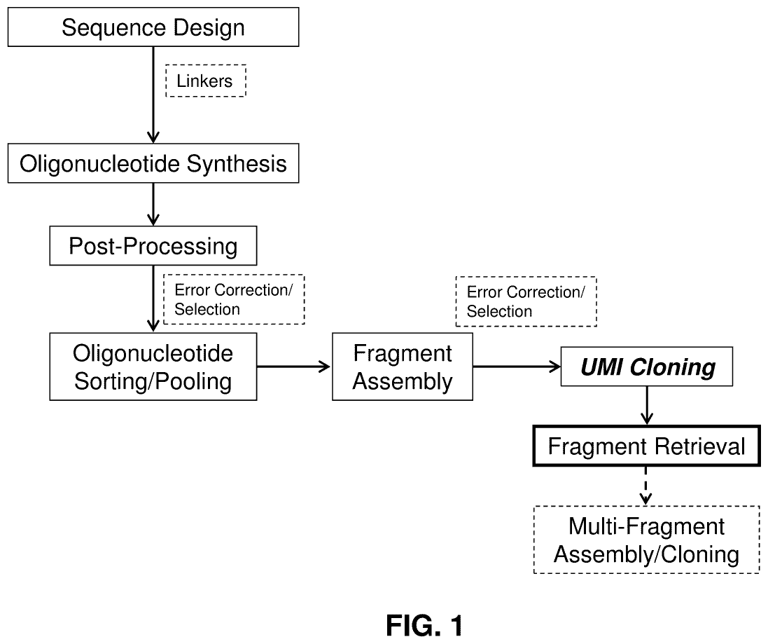

The disclosure relates, in part, to compositions and methods for the identification and retrieval of a desired target nucleic acid from a mixture of nucleic acid molecules. Provided, as examples, are compositions and methods for high throughput synthesis, assembly and isolation of nucleic acid molecules in many instances, with high sequence fidelity. While the disclosure has numerous aspects and variations associated with it, some of these aspects and variations of applicability of the technology may be related to one or more process steps represented in the exemplary workflow shown in FIG. 1.

In this workflow, once a desired nucleic acid molecule (e.g., a nucleic acid fragment) is contemplated, a design path is set up for the production of this nucleic acid molecule. The first step will typically comprise the design of the sequence of the nucleic acid molecule and how it is to be generated. A target sequence may be broken down into smaller fragments, e.g., nucleic acid fragments that may be assembled from chemically synthesized oligonucleotides. Nucleic acid fragment and oligonucleotide design may include sequence optimization (e.g., to allow for efficient production and expression in a target organism) as well as attachment of flanking regions. For example, those oligonucleotides that will form the termini of a nucleic acid assembly product may incorporate linkers with universal primer binding sites and/or restriction enzyme cleavage sites for downstream processing. Following synthesis and processing, oligonucleotides may be sorted and pooled for assembly of longer nucleic acid molecules through a series of assembly reactions, which may then be used as sub-components for the assembly of larger nucleic acid molecules (see, e.g., FIGS. 2 and 3). Typically, nucleic acid amplification reactions (e.g., PCR) are used for the oligonucleotide assembly process. However, in some examples, double-stranded oligonucleotides may also be assembled by ligation.

In some instances, oligonucleotides and/or assembled nucleic acid fragments may be subject to error correction or selection processes. Error correction and/or selection of nucleic acid molecules may occur at one or more points along the workflow.

Typically, assembled nucleic acid molecules are inserted into a vector, then the vector is introduced into a host cell, followed by selection to generate clonal isolates containing candidate inserts representing the desired product nucleic acid molecule. These candidates may then be sequenced to determine (1) whether the desired product nucleic acid molecule is present and, if so, (2) whether the nucleotide sequence of the desired product nucleic acid molecule is correct. Further, a sufficient number of clonal isolates will typically be generated to have a high probability that at least one of the clonal isolates is of the desired product nucleic acid molecule with the correct sequence. To avoid a cell-based cloning procedure, methods disclosed herein may be performed to identify an error-free target nucleic acid fragment in a mixture of nucleic acid molecules. Once a desired nucleic acid fragment is identified, it can be retrieved and used in downstream processes. For example, multiple sequence-verified nucleic acid fragments may be further combined for multi-fragment assembly to obtain longer nucleic acid molecules and optionally cloned.

Methods described herein provide assembled nucleic acid molecules having a high “sequence fidelity”. This high sequence fidelity can be achieved by, for example, one, two or all four of the following: accurate nucleic acid synthesis, selection of oligonucleotides and sub-assembly components having “correct” sequences, error correction, and sequence verification of product nucleic acid molecules. Components and methods for achieving high sequence fidelity will generally be used in more than one portion of workflows and each component or method may be used more than once. For example, the exemplary workflow set out in FIG. 1 shows error correction/correct sequence selection occurring at two different locations in the workflow as further described below.

To identify and retrieve error-free assembled nucleic acid molecules from a mixture of assembled fragments, the exemplary workflow in FIG. 1 comprises a “UMI cloning” procedure that allows for selective retrieval of sequence-verified nucleic acid molecules as described in more detail below. Components and/or methods relating to UMI cloning may be useful, for example, (1) in workflows such as that set out in FIG. 1, (2) in other workflows related to nucleic acid tracing and retrieval, (3) as “stand alone” components and/or methods, and (4) as components and/or methods that may be practiced in combination with other components and/or methods set out herein.

### Sequence Design

As noted above, often, one of the first steps in producing a nucleic acid molecule or protein of interest, after the molecule(s) has been identified, is nucleic acid molecule design. A number of factors go into design of the nucleic acid sequence to be synthesized and the oligonucleotides used to generate the nucleic acid molecule. These factors include one or more of the following: (1) the AT/GC content of all or part of the nucleic acid molecule (e.g., the coding region), (2) the presence or absence of restriction endonuclease cleavage sites (including the addition and/or removal of restriction sites), (3) preferred codon usage for the particular protein production or host expression system that is to be employed, (4) junctions of the oligonucleotides being assembled, (5) the number and lengths of the oligonucleotides used to produce the desired nucleic acid molecule, (6) minimization of undesirable regions (e.g., “hairpin” sequences, regions of sequence homology to cellular nucleic acids, repetitive sequences, inhibitory cis-acting elements, restriction enzyme cleavage sites, internal splice sites etc.) and (7) coding region flanking segments that may be used for attachment of 5′ and 3′ components (e.g., restriction endonuclease sites, primer binding sites, sequencing adapters or barcodes, recombination sites, etc.).

In many instances, parameters will be input into a computer and software will generate an in silico nucleotide sequence that balances the input parameters. The software may place “weights” on the input parameters in that, for example, what is considered to be a nucleic acid molecule that closely matches some of the input criteria may be difficult or impossible to assemble. Exemplary nucleic acid design methods are set out in U.S. Pat. No. 8,224,578.

One main aspect of nucleic acid molecule design is the probability that the nucleic acid molecule can be produced in a “single-run”. Put another way, the probability that a particular synthesis and assembly cycle will result in the generation of the designed nucleic acid molecule. This may be estimated by the use of data derived from past synthesis and assembly cycles and characteristics of the designed nucleic acid molecules. A number of nucleic acid design parameters are set out in Fath et al., PLoS One, 6: e17596 (2011).

As noted herein one important feature of the coding region is codon usage and expression efficiency is also thought to increase with the use of preferred codons for a particular cell type and there is evidence that low-frequency-usage codons within a coding sequence provides for genetic instruction that regulates the rate of protein synthesis. (Reviewed in Angov, “Codon usage: Nature's roadmap to expression and folding of proteins,” Biotechnol. J., 6:650-659 (2011).). Codon usage may be adjusted across a coding region and/or in portions of coding regions to match desired codons. In many instances, this will be done while maintaining a desired level of synthesizability.

Further, nucleic acid molecules design factors may be considered across the length of the nucleic acid molecule or in specific regions of the molecule. For example, GC content may be limited across the length of the nucleic acid molecule to prevent synthesis “failures” resulting from specific locations within the molecule. Thus, synthesizability of the nucleic acid molecule is a characteristic of the entire nucleic acid molecule in that a regional “failure to assemble” results in the designed nucleic acid molecule not being assembled. From a regional perspective, codons may be selected for optimal translation but this may conflict with, for example, region limitation of GC content. A synthetic gene ought therefore to be optimized in relation to the codon usage and the GC content and, on the other hand, substantially avoid the problems associated with DNA motifs and sequence repeats and inverse complementary sequence repeats.

The aim therefore is to reach a compromise which is as optimal as possible between satisfying the various requirements. For this reason, various computer-assisted methods have been proposed for ascertaining an optimal codon sequence. Further factors which may influence the result of expression are DNA motifs and repeats or inverse complementary repeats in the base sequence. Certain base sequences produce in a given organism certain functions which may not be desired within a coding sequence. Examples are cis-active sequence motifs such as splice sites or transcription terminators. The presence of a particular motif may reduce or entirely suppress expression or even have a toxic effect on the host organism. Sequence repeats may lead to lower genetic stability and impede the synthesis of repetitive segments owing to the risk of incorrect hybridizations. Inverse complementary repeats may lead to the formation of unwanted secondary structures at the RNA level or cruciform structures at the DNA level, which impede transcription and lead to genetic instability, or may have an adverse effect on translation efficiency.

The presence of inhibitory or undesired sequence motifs can be established for example with the aid of a variant of a dynamic programming algorithm for generating a local alignment of the mutually similar sequence segments. Such an algorithm is set out in U.S. Patent Publication No. 2007/0141557. Typically, to calculate the criterion weight relating to the repetitive elements, the individual weights of all the local alignments where the alignment weight exceeds a certain threshold value are summed. Addition of these individual weights gives the criterion weight which characterizes the repetitiveness of the test sequence.

Oligonucleotide design may further comprise incorporation of sequence elements required for downstream processing, assembly, sequencing, sequence retrieval, cloning etc. Such elements may be linkers, adapters, PCR handles, barcodes, UMIs, overlaps, primer binding/recognition sites, or restriction enzyme cleavage sites etc., as described in more detail below.

The in silico design of the nucleic acid in terms of synthesizability may also include “fragmentation” of the full-length nucleic acid sequence into smaller fragments (“sub-fragments”) that can be assembled based on single-stranded oligonucleotides. Methods and systems for automated nucleic acid synthesis design are described for example in U.S. Pat. No. 7,164,992. In most instances, oligonucleotides will provide the starting point for the assembly methods underlying the present disclosure.

The sequence design may also take into account requirements for multiplexing of oligonucleotides belonging to different nucleic acid fragments of a product nucleic acid molecule as described in PCT Publication WO 2020/212391.

### Oligonucleotide Synthesis

Oligonucleotides or nucleic acid sub-fragments used for assembly of a desired nucleic acid molecule may be derived from a number of sources, for example, they may be cloned, derived from polymerase chain reactions, chemically synthesized or purchased. In many instances, chemically synthesized nucleic acids tend to be of less than 300 nucleotides in length, more typically up to 200 nucleotides in length. PCR and cloning can be used to generate much longer nucleic acids. Further, the percentage of erroneous bases present in nucleic acids (e.g., nucleic acid fragments) is, to some extent, tied to the method by which it is made. Typically, chemically synthesized nucleic acids have the highest error rate.

A number of methods for chemical synthesis of oligonucleotides are known. In many instances, oligonucleotide synthesis is performed by a stepwise addition of nucleotides to the 5′-end of the growing chain until oligonucleotides of desired length and sequence are obtained. Further, each nucleotide addition can be referred to as a synthesis cycle and often consists of four chemical reactions: (1) De-Blocking/De-Protection, (2) Coupling, (3) Capping, and (4) Oxidation. Chemical oligonucleotide synthesis may be column-based or micro-array/micro-chip-based (for summary and comparison see Hughes and Ellington, 2017, Cold Spring Harb. Perspect. Biol. 2017; 9: a023812). In some instances, oligonucleotides used in workflows described herein may be obtained by synthesizing on a microarray or microchip as disclosed in PCT Publication WO 2016/094512.

A number of variations to the oligonucleotide synthesis process may be made. For example, electrochemically generated acid (EGA) or photogenerated acid (PGA) may be used to remove the protecting group (e.g., DMT) before the next amidite is added to the nucleic acid molecule attached to the solid support. In some examples, at least one proton carrier, such as 2-chloro-6-methylpyridine or diphenylamine, may be present in the solution with the EGA or PGA. The at least one proton carrier may act to reduce the effect of DNA degradation by accepting protons from the EGA or PGA, thereby adjusting the acidity of the solution. EGA and PGA deprotection reagents and methods for generating such acids, as well as their use in oligonucleotide synthesis are set out for example in Maurer et al., “Electrochemically Generated Acid and Its Containment to 100 Micron Reaction Areas for the Production of DNA Microarrays”, PLoS, Issue 1, e34 (2006), or in PCT Publications WO 2013/049227 and WO 2016/094512.

While in many instances oligonucleotides may be produced using phosphoramidite synthesis chemistry, as well as variations thereof, other methods may also be used to produce oligonucleotides, including PCR, restriction enzyme digest, exonuclease treatment, or template-independent synthesis using a nucleotidyl transferase enzyme all of which are contemplated by the methods of the present disclosure. Exemplary methods of template-independent synthesis using a nucleotidyl transferase enzyme are set out in U.S. Pat. No. 8,808,989. The nucleotidyl transferase enzyme (e.g., terminal deoxynucleotidyl transferase) is used to incorporate nucleotide analogs having an unmodified 3′ hydroxyl and a cleavable protecting group. Because of the protecting group, synthesis pauses with the addition of each new base, whereupon the protecting group is cleaved, leaving a polynucleotide that is essentially identical to a naturally occurring nucleotide (i.e., is recognized by the enzyme as a substrate for further nucleotide addition).

Nucleotide triphosphates (e.g., deoxynucleotide triphosphates) (NTPs) suitable for use with template-independent enzymatic oligonucleotide synthesis methods will have protecting groups that do not prevent the NTPs from being used by a nucleotidyl transferase as a substrate and can be efficiently removed to allow for addition to an oligonucleotide chain. Thus, in certain examples, the nucleotide addition occurs via enzymatic reaction. Enzymatic oligonucleotide synthesis may also be conducted in the presence of a support-bound single-strand DNA template using a template-dependent polymerase including DNA polymerases and reverse transcriptase, wherein a surface-bound oligonucleotide is extended by sequential polymerase-based incorporation of reversibly blocked nucleotides. (For further review of chemical and template-independent and template-dependent enzymatic oligonucleotide synthesis methods see Song et al., 2021, Frontiers in Bioengineering and Biotechnology, doi: 10.3389/fbioe.2021.689797). Thus, in certain examples, the disclosure includes methods in which oligonucleotides are produced by chemical or enzymatic reaction.

In some examples of the disclosure, oligonucleotides are attached to one or more solid support (e.g., a magnetic or non-magnetic bead, a matrix, a resin, a gel, the surface of an array or chip etc.). In some examples, oligonucleotides are attached to one or more support via a succinyl linker. In certain examples, a universal linker moiety may be located between the succinyl group and the oligonucleotides. Alternatively, the linker moiety may have a specific base attached as the starting base of the oligonucleotide. In such instances, solid supports (e.g., once having a dA, dT, dC, dG, or dU) are selected based upon the starting base of the oligonucleotide being synthesized.

### Post Processing

Once the oligonucleotide synthesis has been completed, the resulting oligonucleotides are typically subjected to a series of post processing steps that may include one or more of the following: (a) cleavage of the oligonucleotides or elution from the support, (b) concentration measurement, (c) concentration adjustment or dilution of oligonucleotide solutions, often referred to as “normalization”, to obtain equally concentrated dilutions of each oligonucleotide species, and, optionally, (d) pooling or mixing aliquots of two or more normalized oligonucleotide samples to obtain equimolar mixtures of all oligonucleotides required to assemble one or more specific nucleic acid molecules, wherein the aforementioned steps may be combined in different orders.

The method used to cleave or elute the oligonucleotides from a support is often specific for the linking group through which the oligonucleotide is linked to the solid support. Typically, oligonucleotides are cleaved from solid supports using, for example, gaseous ammonia, aqueous ammonium hydroxide, aqueous methylamine, or mixtures thereof. A succinyl linker may be cleaved by the use of, for example, concentrated aqueous ammonium hydroxide. The reaction is usually carried out at temperatures between 50 °C and 80 °C for at least one to about eight hours. In certain examples, the succinyl linker may be cleaved by the use of ammonia gas, using increased heat and pressure, such as, for example, a temperature of about 80 °C, and a pressure of about 3 bars for a time of about 2 hours.

### Oligonucleotide Sorting/Pooling

Microarray or chip synthesized oligonucleotides are attractive for nucleic acid synthesis because of low cost and high throughout. Some chip-based oligonucleotide synthesis platforms allow for the synthesis of up to one million 100mers on a single glass slide. Further, assembly reaction of a typical nucleic acid molecule (e.g., a gene) generally requires a mix of about 20 to 60 (e.g., about 40) oligonucleotides. Thus, assuming only one copy of each 100mer is produced, such slide or array-based oligonucleotide synthesis can be used to generate enough different oligonucleotides for the synthesis of 25,000 nucleic acid molecules.

When a large number of oligonucleotides are produced, for example, on a single surface such as a slide, array or chip, oligonucleotides may be released in bulk by methods such as through the cleavage of a linker and pooled for subsequent assembly steps. Such linkers may be cleaved, for example, photochemically with a laser, electrochemically, or micromechanically.

Where oligonucleotides synthesized on the same microarray or chip are components of different assembly products, selective release of sets of oligonucleotides may be desired. This can be achieved by various means. For example, a defined number of oligonucleotides may be selectively released by amplification off the synthesis support, e.g., by using primers that specifically bind to primer binding sites included in some or all of the oligonucleotides. In instances where oligonucleotides are synthesized on “loose” supports or on a chip-based platform on beads within wells (as described, e.g., in U.S. Pat. Publ. No. 2016/0186166 A1), a selected number of beads carrying oligonucleotides comprising a pre-defined sequence may be selectively released, e.g., using a micropipette, by suction, magnetic attraction or by gas bubble displacement, as described in U.S. Pat. Publ. No. 2016/0186166 A1. For example, in a first step, all beads carrying oligonucleotides that belong to a first assembly product, may be simultaneously released and pooled into a first vessels (such as a well of a multiwell plate). In a second step, all beads carrying oligonucleotides that belong to a second assembly product, may be simultaneously released and pooled into a second vessel or well of a multiwell plate.

Once a subset of oligonucleotides is released from a synthesis array or chip, the released oligonucleotides may be collected and subjected to one or more assembly reactions to generate a single assembly product or a known number of assembly products. The pooling of the oligonucleotides may be achieved by any known method including for example the use of a collection device described in PCT Publication WO 2016/094512.

Pooled oligonucleotides may be components of a single nucleic acid fragment to be assembled or may be components of multiple nucleic acid fragments to be assembled simultaneously in a single reaction (“multiplex assembly”).

One approach for addressing the complexity issue in nucleic acid assembly is to release and/or collect oligonucleotides that are components of a limited number of assembly products (e.g., from about 1 to about 20, from about 2 to about 20, from about 3 to about 20, from about 4 to about 20, from about 1 to about 3, from about 1 to about 5, from about 1 to about 10, etc. assembly products) to keep oligonucleotide complexity down in assembly mixtures.

The efficiency of this process may be improved in a number of ways that begin with the generation of oligonucleotide pools for the formation of more than one assembly product (a sub-fragment) in a single reaction vessel (e.g., a tube, a well of a microwell plate, etc.). For example, the oligonucleotide pool may contain oligonucleotides for the formation of multiple sub-fragments but only a sub-set of these sub-fragments may be generated. One way of doing this is illustrated in FIG. 3.

The left side of FIG. 3, Tube A, shows a schematic representation of a workflow for the release of oligonucleotides from beads for the formation of a pool, where the oligonucleotides are components of a single sub-fragment, as well as the generation of the single sub-fragment by PCR. The right side of FIG. 3, Tube B, shows a schematic representation of a workflow for the release of oligonucleotides from beads for the formation of a pool, where the oligonucleotides are components of four sub-fragments, as well as the generation of the four sub-fragments by PCR. This allows for the generation of four nucleic acid molecules (having different sequences) in a single vessel and through the same PCR cycling processes. Such processes allow for the collection of oligonucleotides that are components of more than one nucleic acid molecule. This improves the efficiency of nucleic acid molecule assembly in that more than one nucleic acid molecule may be produced without sorting of oligonucleotide components.

In some instances, the number of nucleic acid molecules represented by oligonucleotides in a pool may be so large that assembly of all of the nucleic acid molecules may not occur efficiently. In certain examples it may be desired to reduce the complexity of an oligonucleotide pool to a level that allows for simultaneous assembly of multiple sub-fragments in a single reaction but is sufficiently low to guarantee efficient assembly without cross-hybridization events between oligonucleotides belonging to different sub-fragments. For example, instead of successively releasing oligonucleotide pools for the assembly of 100-200 sub-fragments, 10 to 100 pools each comprising the oligonucleotides for assembly of 2 to 10 sub-fragments could be generated simultaneously. Exemplary methods and rules for designing, grouping and pooling of nucleic acid molecules for efficient multiplex assembly are disclosed in WO 2020/212391 A1, which is incorporated herein by reference.

In some instances the pooled oligonucleotides may be designed such that all nucleic acid fragments assembled in the same reaction mixture comprise substantially identical 5′- and/or 3′-ends also referred to as “linkers”, which may contain functional elements such as universal primer binding sites and/or restriction enzyme cleavage sites or homologous regions, allowing for simultaneous amplification with a universal pair of primers in a subsequent PCR reaction and/or simultaneous processing in a downstream assembly or cloning step. Thus, in some instances an oligonucleotide belonging to a first fragment may have the same or substantially the same 5′end as an oligonucleotide belonging to a different second fragment. Likewise, in some instances an oligonucleotide belonging to a first fragment may have the same or substantially the same 3′end as an oligonucleotide belonging to a different second fragment.

It will be understood that “substantially identical” as used herein means that, although sequences are intended to be identical, there may be some errors in synthesized nucleic acid molecules resulting in less than 100% identity. For example, two molecules can be considered substantially identical, if the percent identity between the two molecules is at least 75%, 80%, 85%, 90%, 95%, 98%, 99%, 99.9% or greater, when optimally aligned.

In some instances, each fragment assembled from a sub-set of oligonucleotides comprises a universal primer binding site and/or a restriction enzyme cleavage site. The universal primer binding site may be used in downstream workflows for simultaneous amplification of all assembled fragments. For example, where fragments will be tagged for sequencing or UMI cloning as described in more detail below, a universal primer binding site can be used to conjugate barcodes and/or sequencing adapters via fusion PCR. Universal primer binding sites may also be used for normalizing the nucleic acid fragments in a mixed pool of sub-fragments (e.g., prior to a tagging step). In such instance, a set of diverse primer binding sites may be used.

### Fragment Assembly

The pooled oligonucleotides will typically be assembled into larger nucleic acid molecules (also referred herein as nucleic acid fragments) in a stepwise manner and optionally, amplified.

One method for assembling nucleic acid fragments from oligonucleotides is depicted in FIG. 2 and involves starting with oligonucleotides that will generally contain sequences that are overlapping at their termini which are “stitched” together via these complementary sequence regions using PCR. In some examples, the overlaps are approximately 10 base pairs; in other examples, the overlaps may be 15, 25, 30, 50, 60, 70, 80 or 100 base pairs, etc. (e.g., from about 10 to about 120, from about 15 to about 120, from about 20 to about 120, from about 25 to about 120, from about 30 to about 120, from about 40 to about 120, from about 10 to about 40, from about 15 to about 50, from about 40 to about 80, from about 60 to about 90, from about 20 to about 50, from about 15 to about 35, etc. base pairs). In order to avoid mis-assembly, individual overlaps will typically not be duplicated or closely matched amongst the assembly products. Since hybridization does not require 100% sequence identity between the participating nucleic acid molecules or regions, each terminus should be sufficiently different to prevent mis-assembly. Further, termini intended to undergo homologous recombination with each other should share at least 90%, 93%, 95%, or 98% sequence identity. Further, multiple cycles of polymerase chain reactions may be used to generate successively larger nucleic acid molecules.

An alternative method for PCR-based assembly of nucleic acid molecules (e.g., chemically synthesized nucleic acid molecules) is based on the direct ligation of overlapping pairs of 5′-phosphorylated oligonucleotides (“ligation-based assembly”). In this process, single-stranded oligonucleotides are synthesized, phosphorylated and annealed to form double-stranded oligonucleotides with complementary overhangs (e.g., overhangs of four nucleotides). The individual double-stranded molecules are then ligated to each other to form larger constructs. In certain embodiments this method may be desirable over PCR methods in particular where highly repetitive sequences, such as GC stretches are to be assembled. This method may be used to assemble from about two to about forty nucleic acid molecules (e.g., from about two to about forty, from about three to about forty, from about five to about forty, from about eight to about forty, from about two to about thirty, from about two to about twenty, from about two to about ten, etc. nucleic acid molecules). A related method is described in U.S. Pat. No. 4,652,639, the disclosure of which is incorporated herein by reference.

In alternative ligation-based assembly, double-stranded oligonucleotides may be obtained by amplification of single-stranded oligonucleotides which may contain flanking primer binding sites and restriction enzyme recognition sites near their termini. These double-stranded oligonucleotides may then be treated with one of more suitable restriction enzymes to generate, for example, either one or two “sticky ends” for subsequent hybridization and ligation. In some instances, the double-stranded oligonucleotides may contain type IIS restriction enzyme recognition sites and be assembled using concurrent cleavage and ligation, also referred to as “Golden Gate” assembly. Golden Gate assembly is a one-tube efficient cloning method based on type IIs restriction enzymes (such as BsmBI, BbsI, BsaI, AarI, SapI, BtgZI, etc.) that cleave outside their recognition sites and typically leave 4-base overhangs. For this purpose, one or more (e.g., 2, 3, 4, 5, 6, 7, 8, 9 or 10 etc.) linear double-stranded fragments (e.g. amplified oligonucleotides) comprising parts A, B, C etc., may be combined and cleaved with one or more type IIs restriction enzymes to generate compatible overhangs between part A and part B, and between part B and part C to allow for assembly of all parts in a seamless and oriented manner. The reaction mixture may also comprise a ligase (e.g., a T4 DNA ligase or a Taq ligase) for ligation of the assembled parts.

Other methods of nucleic acid assembly include those described in U.S. Patent Publication Nos. 2010/0062495 A1; 2007/0292954 A1; 2003/0152984 AA; and 2006/0115850 AA and in U.S. Pat. Nos. 6,083,726; 6,110,668; 5,624,827; 6,521,427; 5,869,644; 6,472,184 and 6,495,318.

In some aspects, lengths of nucleic acid fragments assembled from oligonucleotides will vary from about 50 base pairs to about 5,000 base pairs, from about 100 base pairs to about 2,000 base pairs, from about 150 base pairs to about 1,000 base pairs or from about 200 bp to about 500 bp, but even longer nucleic acid fragments may be assembled from oligonucleotides depending on the oligonucleotide length and assembly method.

To limit the amount of errors introduced during PCR-based assembly reactions, the overlap extension PCR reaction may be performed using a high fidelity polymerase with 3′-5′ exonuclease proofreading activity. A non-exhaustive list of suitable polymerases includes Pfu DNA polymerase (Thermo Fisher Scientific), DEEP VENT® DNA polymerase (New England Biolabs), Q5® High-Fidelity DNA Polymerase (New England Biolabs), PHUSION® (New England Biolabs), PHUSION® Hot Start Flex (New England Biolabs), PRIMESTAR® HS (TAKARA), PRIMESTAR® GXL (TAKARA), PRIMESTAR® Max (TAKARA), ACCUPRIME™ Pfx (Thermo Fisher Scientific), PLATINUM™ DNA Polymerase High Fidelity (Thermo Fisher Scientific), PHUSION® Flash II DNA Polymerase (Thermo Fisher Scientific), PHUSION® Hot Start II High-Fidelity DNA Polymerase (Thermo Fisher Scientific), ACCURA® High-Fidelity polymerase (Lucigen), IPROOF™ High-Fidelity polymerase (Bio-Rad), PAN PowerScript DNA Polymerase (PAN Biotech) or TRUESCRIPT™ DNA polymerase (PAN Biotech).

### Error Correction/Selection

As illustrated in FIG. 1, error correction and/or selection procedures may be employed at various steps of the disclosed workflows to increase sequence fidelity. High sequence fidelity can be achieved by several means, including sequencing of partially assembled or fully assembled nucleic acid molecules to identify ones with correct sequences

Errors may find their way into nucleic acid molecules in a number of ways. Examples of such ways include chemical synthesis errors, amplification/polymerase mediated errors (especially when non-proof reading polymerases are used), and assembly mediated errors (usually occurring at nucleic acid fragment junctions).

The assembled nucleic acid molecule may be a composite in the sense that all the components (e.g., oligonucleotides) are synthesized and assembled or only some of the components are synthesized and these components are then assembled with other nucleic acid molecules (e.g., nucleic acid fragments generated by PCR or nucleic acid molecules propagated within cells). However, for purposes of illustration, consider the situation where one hundred nucleic acid molecules are to be assembled, each molecule is one hundred base pairs in length and there is one error per 200 base pairs. The net result is that there will be, on average, 50 sequence errors in each 10,000 base pair assembled nucleic acid molecule. If one intends, for example, to express one or more proteins from the assembled nucleic acid molecule, then the number of amino acid sequence errors would likely be considered to be too high. Further, a number of the protein coding region nucleotide sequence errors will result in “frameshift” mutations yielding proteins that will generally not be desired. Also, non-frameshift coding regions may result in the formation of proteins with point mutations. All of these will “dilute the purity” of the desired protein expression product and many of the produced “contaminant” proteins will be carried over into the final expression product mixture even if affinity purification is employed.

Sequence errors in nucleic acid molecules may be referenced in a number of ways. As examples, there is the error rate associated with the synthesis nucleic acid molecules, the error rate associated with nucleic acid molecules after error correct and/or the selection, and the error rate associated with end product nucleic acid molecules (e.g., error rates of (1) synthetic nucleic acid molecules that have either been selected for the correct sequence or (2) assembled chemically synthesized nucleic acid molecules). These errors may come from the chemical synthesis process, assembly processes, and/or amplification processes. Errors may be removed or prevented by methods, such as, the selection of nucleic acid molecules having correct sequences, error correction, and/or improved chemical synthesis methods.

In some instances, methods of the disclosure will combine error removal and prevention methods to produce nucleic acid molecules with relative low numbers of errors. Thus, assembled nucleic acid molecules produced by methods of the disclosure may have error rates from about 1 base in 1,500 to about 1 base in 30,000, from about 1 base in 2,000 to about 1 base in 30,000, from about 1 base in 4,000 to about 1 base in 30,000, from about 1 base in 8,000 to about 1 base in 30,000, from about 1 base in 10,000 to about 1 base in 30,000, from about 1 base in 15,000 to about 1 base in 30,000, from about 1 base in 10,000 to about 1 base in 20,000, etc.

Two ways to lower the number of errors in assembled nucleic acid molecules is by (1) selection of nucleic acid molecules (oligonucleotides, assembled fragments etc.) for assembly with corrects sequences and (2) correction of errors in nucleic acid molecules, partially assembled sub-assemblies, or fully assembled nucleic acid molecules.

In most instances, regardless of the method by which a larger nucleic acid molecule is generated from chemically synthesized oligonucleotides, errors from the chemical synthesis process will be present. While sequencing of individual nucleic acid molecules may be performed to identify and select error-free nucleic acid molecules as described in more detail below, alternative approaches may comprise one or more error correction or removal steps. Typically, such error removal steps will be performed after a first round of assembly. Thus, in one aspect, methods of the disclosure involve the following (in this order or different orders): (i) fragment amplification and/or assembly (e.g., according to the methods described herein), (ii) error correction or selection, (iii) final assembly (e.g., according to the in vitro or in vivo methods described herein).

In many instances enzymatic error correction may be employed. Once assembled, nucleic acid fragments may undergo mismatch recognition-based error correction in the absence of assembly PCR or amplification. This will often be done by heat denaturation of the subject nucleic acid molecules, followed by renaturation of the nucleic acid molecules which are then and contacted with one or more mismatch recognition proteins. In some examples, mismatch endonucleases (MME) may be used to cleave mismatches in reannealed nucleic acid molecules followed by PCR-based assembly of the cleaved fragments as described, for example, in PCT Publication Nos. WO 2005/095605 A1 or WO 2016/094512 A1. The error can either be removed by using a combination of nucleases with mismatch cleavage and exonuclease activities or by the intrinsic exonuclease activity of the DNA polymerase during assembly of the cleaved fragments. This principle is outlined, e.g., in Saaem et al. (“Error correction of microchip synthesized genes using Surveyor nuclease”, Nucl. Acids Res., 40: e23 (2012)). Such final assembly step may be performed in the presence of terminal primers thereby including functionalities required for downstream processes such as cloning or protein expression. A respective PCR reaction may be set up to first allow the error-corrected fragments to assemble by overlap extension to the full-length in about 15 cycles of denaturation, annealing and extension in the absence of the terminal primers, followed by additional 20 cycles in the presence of the terminal primers.

In many instances, one or more ligase may be present in reaction during error correction. It is believed that some endonucleases used in error correction processes have nickase activity. The inclusion of one or more ligase is believed to seal nicks caused by such enzymes and increase the yield of error corrected nucleic acid molecules after amplification. Exemplary ligases that may be used are T4 DNA ligase, Taq ligase, and PBCV-1 DNA ligase. Ligases used in the practice of the disclosure may be thermolabile or thermostable (e.g., Taq ligase). If a thermolabile ligase is employed, it will typically need to be added to a reaction mixture for each error correction cycle. Thermostable ligases will typically not need to be re-added during each cycle, so long as the temperature is kept below their denaturation point.

One exemplary process for error correction that may be used in methods disclosed herein is also set out in U.S. Pat. No. 7,704,690. Another process for effectuating error correction in chemically synthesized nucleic acid molecules that may be used in methods of the disclosure is by a commercial process referred to as ERRASE™ (Novici Biotech). Yet another process for reducing errors during nucleic acid synthesis that may be used in aspects of the disclosure is referred to as Circular Assembly Amplification and described in PCT Publication WO 2008/112683.

Synthetically generated nucleic acid molecules typically have error rate of about 1 base in 300-500 bases. Conditions can be adjusted so that synthesis errors are substantially lower than 1 base in 300-500 bases. Further, in many instances, greater than 80% of errors are single base frame shift deletions and insertions. Also, less than 2% of errors result from the action of polymerases when high fidelity PCR amplification is employed. Therefore, error-correction processes using PCR-based assembly steps as described above may be combined with one or more error-correction methods not involving polymerase activity. In many instances, mismatch endonuclease correction will be performed using fixed protein:DNA ratio.

In some examples, enzymatic error correction may occur during oligonucleotide assembly or amplification in the presence of thermostable mismatch recognition enzymes as described in PCT Publication No. WO 2021/178809 or in WO 2021/187554 which are incorporated herein by reference.

Exemplary assembly and amplification error correction workflows may be performed using both thermostable and thermolabile mismatch recognition enzymes. By way of example, oligonucleotide assembly may be performed in the presence of one or more (e.g., one, two, three, etc.) thermostable mismatch recognition enzyme (e.g., TkoEndoMS); followed, optionally, by amplification also in the presence of one or more thermostable mismatch recognition enzyme (e.g., TkoEndoMS and/or PfuEndoMS); followed by amplification also in the presence of one or more thermolabile mismatch recognition enzyme (e.g., T7 endonuclease I).

Examples of thermostable mismatch recognition enzymes include Thermococcus kodakarensis mismatch recognition enzyme, abbreviated “TkoEndoMS” (Ishino et al., Nucl. Acids Res. 44:2977-2989 (2016) and Pyrococcus furiosus mismatch recognition enzyme, abbreviated “PfuEndoMS”. A number of additional thermostable mismatch recognition enzymes are set out in PCT Publication Nos. WO 2021/178809 and WO 2021/187554.

Examples of thermolabile mismatch recognition enzymes include T7 endonuclease I, CEL II nuclease, CEL I nuclease, and T4 endonuclease VII.

Non-PCR-based error correction may, e.g., be achieved by separating nucleic acid molecules with mismatches from those without mismatches by binding with a mismatch binding agent in a number of ways. For example, mixtures of nucleic acid molecules, some having mismatches, may be (1) passed through a column containing a bound mismatch binding protein or (2) contacted with a surface (e.g., a bead (such as a magnetic bead), plate surface, etc.) to which a mismatch binding protein is bound.

Exemplary formats and associated methods involve those using surfaces or supports (e.g., beads) to which a mismatch binding protein is bound. For example, a solution of nucleic acid molecules may be contacted with beads to which is bound a mismatch binding protein. One mismatch binding protein that may be used in various aspects of the disclosure is MutS from Thermus aquaticus the gene sequence of which is published in Biswas and Hsieh, J. Biol. Chem. 271:5040-5048 (1996) and is available in GenBank, accession number U33117. Furthermore, mismatch cleavage endonucleases such as T7 endonuclease I or Cel I from, for example, celery may be genetically engineered to inactivate the cleavage function for use in error filtration processes based on mismatch binding. Nucleic acid molecules that are bound to a mismatch binding protein may either be actively removed from a pool of nucleic acid molecules (e.g., via magnetic force where magnetic beads coated with mismatch binding proteins are used) or may be immobilized or linked to a surface such that they remain in the sample whereas unbound nucleic acids are removed or transferred (e.g., by pipetting, acoustic liquid handling etc.) from the sample. Such examples are set out, for example, in PCT Publication WO 2016/094512.

Error correction may also be performed using simultaneously or in separate steps two or more different mismatch recognition enzymes (thermostable and/or thermolabile). One advantage of this is that different mismatch recognition enzymes can vary in their activities towards different type of errors. In some embodiments, Thus, error correction may be performed using two or more mismatch recognition enzymes, where each of the mismatch recognition enzymes recognize different types of errors with different levels of enzymatic activity.

Error correction methods and reagents suitable for use in methods of the disclosure are set out in U.S. Pat. Nos. 7,838,210 and 7,833,759, U.S. Patent Publication No. 2008/0145913 (mismatch endonucleases), PCT Publication WO 2011/102802, and in Ma et al., Trends in Biotechnology, 30 (3): 147-154 (2012). Furthermore, the skilled person will recognize that other methods of error correction and/or error filtration (i.e., specifically removing error-containing molecules) may be practiced in certain examples of the disclosure such as those described, for example, in U.S. Patent Publication Nos. 2006/0127920, 2007/0231805, 2010/0216648, or 2011/0124049.

Once nucleic acid molecules are assembled and optionally error-corrected, their sequences may be verified to confirm that “junction” sequences are correct and that no other nucleotide sequence “errors” are located within assembled nucleic acid molecules.

### UMI Cloning

Sequence-verified nucleic acid molecules may be retrieved by various means.

Gene assembly workflows are typically completed by identifying a clone comprising the desired polynucleotide. For this purpose, assembled polynucleotides are often transformed into E. coli followed by several hours (often overnight) of incubation to allow for growth of transformed bacteria to colonies of visible size. Colony PCR may then be performed to identify clones comprising correctly assembled polynucleotides having the desired length and isolated clones may further be sequenced (e.g., by Sanger sequencing techniques) to identify error-free polynucleotides. Such cloning-based workflows are tedious and slow and present a bottleneck for high-throughput, automated and inexpensive genome construction. New technologies have therefore been developed based on a combination of next generation sequencing and “in vitro cloning” to allow for massive multiplexing of gene or genome synthesis.

Apart from using sequence analysis solely for the (final) verification of isolated clones, sequencing may also be used as an alternative to error correction to identify individual error-free molecules within a pool of nucleic acids and selectively retrieve the error-free nucleic acid molecules. Sequence verification prior to (full-length) nucleic acid assembly may be useful to avoid the carry-over of errors from chemically synthesized nucleic acid molecules into amplification reactions thereby multiplying the errors in assembled polynucleotides. This may be done for example by sequencing a pool of amplified nucleic acid molecules to determine if any errors are present and retrieving one or more error-free molecules for downstream processing. Thus, sequencing techniques may be applied to identify and select error-free nucleic acid molecules for amplification and subsequent assembly.

One method of selective retrieval of desired nucleic acids is referred to as “laser catapulting” which relies on the use of high-speed laser pulses to eject selected clonal nucleic acid populations from a sequencing plate. This method is described, for example, in U.S. Patent Publication No. 2014/0155297.

Another method often referred to as “dial-out PCR” is based on massive parallel sequencing of a complex mixture of nucleic acid fragments modified with unique flanking tags followed by retrieval of error-free nucleic acid molecules from the library by performing a PCR reaction with tag-directed primers for subsequent gene assembly. Dial-out PCR is described e.g., in U.S. Patent Publication No. 2012/0283110, 2012/0322681 or 2014/0141982.

For dial-out PCR, the desired molecule needs to be amplified from a complex mixture of nucleic acid molecules wherein the generated clonal populations are not kept separate during the amplification. For retrieving required nucleic acid molecules, desired nucleic acid fragments need to be selectively amplified from the complex nucleic acids mixture using barcode primers directed to the tags of sequence-verified fragments, which is an extra step that may result in additional errors introduced by amplification. Also, a large library of amplification primers needs to be synthesized and maintained (if barcodes are known/have been designed) or synthesized for each retrieval (if barcodes are random sequences).

Other methods for retrieving sequence-verified nucleic acid molecules are set out in U.S. Pat. No. 8,173,368. The described method comprises the steps of monoclonizing nucleic acids from a mixture of different nucleic acid molecules, parallel sequencing of the monoclonized nucleic acids, identifying and localizing an individual nucleic acid with a desired sequence, and isolating the individual nucleic acid with the desired sequence for further processing. Localization of the desired sequence is affected by immobilization of the support during sequencing and molecules having the desired sequence are then removed directly from the sequencing reaction support. Retrieval of the selected nucleic acids may be accomplished by isolating of beads comprising the selected nucleic acid, cleaving off the nucleic acids from the respective support on which they are immobilized, selective amplification by spatially-resolved addition of PCR reagents or elution by a laser capture method.

The aforementioned method localizes error-free nucleic acids by sequencing and retrieves the sequenced molecules from the sequencing chip (either directly or by generating further copies via PCR amplification). Using only the quantity of sequenced molecules released from the support may not yield sufficient amounts of nucleic acid molecules for subsequent assembly. Sequence-verified molecules are therefore typically further amplified (either in solution upon release or directly off the support) which bears the risk of re-introducing errors. In particular, complex sequence templates comprising repetitive motifs, high GC content, long GC- or AT-stretches, hairpin structures etc., may be prone to erroneous PCR amplification due to polymerase-based substitution errors. Even in the presence of a high fidelity polymerase with very low substitution rates, amplification products suffer from DNA damage introduced during temperature cycling thus compromising the reliability of sequencing or “QC” data that were obtained prior to the amplification step. However, retrieval of selected molecules by other means directly from the sequencing platform may be tedious and limited to a specific sequencing platform or require additional equipment (such as lasers).

The inventors have developed an improved and more flexible workflow referred to as “UMI cloning” or “in vitro cloning herein, that is compatible with any sequencing platform or workflow that allows for clonal amplification of nucleic acid templates in separate compartments and sequencing to identify error-free, in vitro-cloned nucleic acid fragments that can be directly assembled into larger constructs.

In some aspects, a UMI cloning method for identifying a nucleic acid molecule having a desired sequence in a mixture of nucleic acid molecules comprises the following steps:
(a) providing one or more mixtures of nucleic acid molecules, each mixture comprising a plurality of nucleic acid molecules designed to have a desired sequence, wherein each nucleic acid molecule optionally comprises a linker at the 5′ end and at the 3′ end;
(b) providing a set of nucleic acid tags, each tag comprising at least (i) a barcode, and (ii) a handle or adapter at the 5′ end;
(c) determining the concentration of nucleic acid molecules in each of the one or more mixtures;
(d) providing one or more first compartments and diluting each of the one or more mixtures of nucleic acid molecules in a separate first compartment to obtain diluted mixtures of nucleic acid molecules, each diluted mixture comprising a predetermined amount of nucleic acid molecules;
(e) contacting the diluted mixtures of nucleic acid molecules in one or more of the first compartments with the set of nucleic acid tags and attaching a tag to both ends of substantially each nucleic acid molecule in the one or more first compartments to obtain tagged nucleic acid molecules having a barcode region and a handle or adapter region at both ends;
(f) optionally providing one or more pairs of amplification primers designed to hybridize to both ends of the tagged nucleic acid molecules and amplifying the tagged nucleic acid molecules in the one or more first compartments;
(g) optionally purifying the amplified nucleic acid molecules;
(h) providing a set of barcode primers, each barcode primer of the set comprising a barcode-specific region designed to hybridize to a specific barcode in the set of nucleic acid tags;
(i) providing one or more second compartments and contacting at least a portion of each of the amplified and optionally purified tagged nucleic acid molecules with a defined pair of the set of barcode primers in a second compartment and performing an amplification reaction in each second compartment to obtain one or more second compartments comprising: (i) one specific amplification product, (ii) no specific amplification product, and/or (iii) two or more specific amplification products;
(j) optionally identifying second compartments (ii) having no specific amplification product and/or second compartments (iii) having two or more specific amplification products and excluding such second compartments from subsequent steps,
(k) optionally pooling at least a portion of the amplification products from two or more of the second compartments;
(l) sequencing the amplification products to obtain sequence data;
(m) analysing the sequence data, and
(n) identifying one or more second compartments comprising one specific amplification product, thereby identifying one or more nucleic acid molecules having the desired sequence.

In some aspects, a UMI cloning method for identifying a nucleic acid molecule having a desired sequence in a mixture of nucleic acid molecules comprises the following steps:
(a) providing one or more mixtures of nucleic acid molecules, each mixture comprising a plurality of nucleic acid molecules designed to have a desired sequence, wherein each nucleic acid molecule optionally comprises a linker at the 5′ end and at the 3′ end;
(b) providing a set of nucleic acid tags, each tag comprising at least (i) a barcode, and (ii) a handle or adapter at the 5′ end;
(c) contacting the one or more mixtures of nucleic acid molecules with the set of nucleic acid tags and attaching a tag to both ends of substantially each nucleic acid molecule to obtain tagged nucleic acid molecules having a barcode region and a handle or adapter region at both ends;
(d) optionally determining the concentration of tagged nucleic acid molecules in one or more mixtures;
(e) providing one or more first compartments and diluting each of the one or more mixtures of tagged nucleic acid molecules in a separate first compartment to obtain diluted mixtures of tagged nucleic acid molecules, each diluted mixture comprising a predetermined amount of tagged nucleic acid molecules;
(f) optionally providing one or more pairs of amplification primers designed to hybridize to both ends of the tagged nucleic acid molecules and amplifying the tagged nucleic acid molecules in the one or more first compartments;
(g) optionally purifying the amplified nucleic acid molecules;
(h) providing a set of barcode primers, each barcode primer of the set comprising a barcode-specific region designed to hybridize to a specific barcode in the set of nucleic acid tags;
(i) providing one or more second compartments and contacting at least a portion of each of the optionally amplified and optionally purified tagged nucleic acid molecules with a defined pair of the set of barcode primers in a second compartment and performing an amplification reaction in each second compartment to obtain one or more second compartments comprising: (i) one specific amplification product, (ii) no specific amplification product, and/or (iii) two or more specific amplification products;
(j) optionally identifying second compartments (ii) having no specific amplification product and/or second compartments (iii) having two or more specific amplification products and excluding such second compartments from subsequent steps;
(k) optionally pooling at least a portion of the amplification products from two or more of the second compartments;
(l) sequencing the amplification products to obtain sequence data;
(m) analysing the sequence data, and
(n) identifying one or more second compartments comprising one specific amplification product, thereby identifying one or more nucleic acid molecules having the desired sequence.

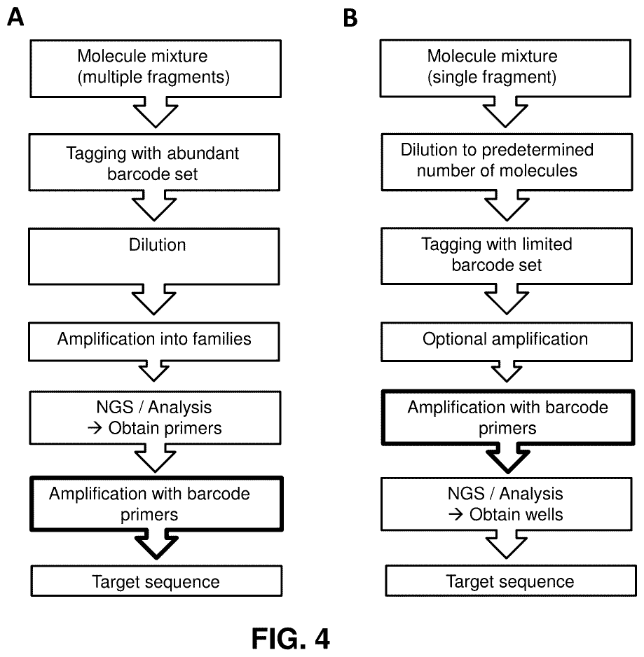

Various embodiments of this method are illustrated in FIGS. 4B, 5, 6, 8, 9, 11 and 12 and individual steps and variations of the method are now described in more detail.

In step (a) one or more mixtures of nucleic acid molecules are provided, wherein each mixture comprises a plurality of nucleic acid molecules designed to have a desired sequence.

The nucleic acid molecules may be double-stranded or at least partially double-stranded. In some instances, the nucleic acid molecules may be provided as linear double-stranded molecules. In some embodiments, the nucleic acid molecules may be designed or optimized for expression in a target host as described above. In many instances the nucleic acid molecules may comprise a GC content of from about 35% to about 60% or from about 40% to about 55%.

In some instances, prior to step (a) the nucleic acid molecules may have been subject to one or more error reduction or error correction steps as outlined above. Such error correction or reduction may for example be performed during or after assembling the nucleic acid molecules. In some embodiments, the nucleic acid molecules in each mixture may have an error rate of less than 1 in 400 bp, preferably less than 1 in 800 bp, more preferably less than 1 in 1,000 bp. In some instances, the error rate may be less than 1 on 2,000 or less than 1 in 5,000. Low error rates may be achieved by applying any of the error correction or selection methods described above.

Typically, the nucleic acid molecules have lengths of at least 150 nucleotides or bp, more preferably more than 200 nucleotides or bp (e.g., between about 200 and about 5,000 nucleotides or between about 200 and about 500 bp). In some instances, the nucleic acid molecules may have lengths of more than 500 bp, more than 600 bp, more than 700 bp, more than 800 bp, more than 900 bp, more than 1,000 bp, more than 2,000 bp or more than 5,000 bp. In some examples the nucleic acid molecules may have lengths of between about 400 bp and about 2,000 bp or between about 500 bp and about 10,000 bp or between about 1,000 bp and about 50,000 bp or between about 2,000 bp and about 100,000 bp. In many instances the nucleic acid molecules in a given mixture will have equal or nearly equal lengths (i.e., not deviate more than about 5%, preferably not more than about 2%, preferably not more than about 1% in length). Nucleic acid molecules in different mixtures may have different lengths. For example, nucleic acid molecules may typically be between about 200 and about 5,000 base pairs in length, or between about 400 and about 1,200 bp in length. In some instances, nucleic acid lengths in different mixtures may vary less than 20% or less than 10%.

The nucleic acid molecules may have been derived from naturally occurring nucleic acid molecules (e.g., by PCR amplification of genomic DNA regions) or may have been assembled from a plurality of oligonucleotides. For example, the nucleic acid molecules may be obtained by PCR assembly of overlapping single-stranded oligonucleotides as described above and shown in FIG. 2. Alternatively, the nucleic acid molecules may be obtained by ligation of double-stranded oligonucleotides. Ligation-based assembly may be preferred in certain instances where amplification is to be avoided (e.g., where oligonucleotides may have repetitive sequences such as GC stretches). The oligonucleotides from which the nucleic acid molecules are assembled, may be synthesized by any means, including chemical, electrochemical, photochemical or enzymatic synthesis as described above in more detail. Solid supports for oligonucleotide synthesis may have various forms as described above and may comprise the surface of a microchip or a microarray or may comprise particles or beads. Depending on the underlying production platform or technique (i.e., chemical, enzymatical etc.) oligonucleotides may have different lengths but will typically be between about 15 and about 300 nucleotides, preferably between about 20 and about 200 nucleotides in length.

Each nucleic acid molecule in the mixture may comprise a “linker” at the 5′ end and at the 3′ end. The term “linker”, “linker region”, “linker sequence” etc., as used herein, refers to a sequence region that confers one or more universal functionalities to a set of nucleic acid molecules. Linkers may be between about 10 and about 50 nucleotides in length. In addition, a linker may comprise a universal primer binding site designed for PCR-based attachment of a nucleic acid tag or adapter molecule having a linker-complementary sequence region. In some examples, the universal primer binding site of a linker may be between about 10 and about 30 nucleotides in length. Further, the universal primer binding site of a linker flanking a nucleic acid molecule at the 5′ end may differ in sequence from the universal primer binding site of a linker flanking the nucleic acid molecule at the 3′ end. Universal primer binding sites at the 5′ and the 3′ ends of nucleic acid molecules in the mixture should be sufficiently different to allow specific amplification with distinct universal primer pairs as set out below.

In some instances, a linker may further comprise one or more restriction enzyme cleavage sites designed and positioned for subsequent cleavage or removal of a flanking (universal) primer binding site, a barcode, a tag, an adapter etc. In some examples, a linker can include any type of restriction enzyme recognition sequence, including type I, type II, type IIs, type IIB, type III, type IV restriction enzyme recognition sequences, or recognition sequences having palindromic or non-palindromic recognition sequences. In a preferred embodiment, a linker may comprise a type IIs restriction enzyme cleavage site to provide overhangs for seamless oriented assembly, also referred to as “Golden Gate” cloning as described below in more detail. In some instances, linkers may also contain one or more uracils for subsequent removal of flanking regions with a uracil glycosylase as described elsewhere herein.

The method provides for one or more mixtures of nucleic acid molecules, preferably at least two mixtures of nucleic acid molecules, more preferably at least three mixtures of nucleic acid molecules. The number of mixtures of nucleic acid molecules to be processed in parallel may vary but will typically be between 2 and about 1,000, between about 10 and about 500, between about 30 and about 300, or between about 50 and about 200.

In step (b) a set of nucleic acid tags is provided. The term “tag” as used herein refers to a nucleic acid segment that comprises one or more sequence elements that confer a certain functionality to a target nucleic acid molecule and can be attached to one or both ends of a nucleic acid molecule e.g., by PCR or ligation. Tags will typically comprise non-coding sequence elements required for nucleic acid analysis, capture, tracking, sorting and the like. In many instances a tag comprises at least a barcode and a handle as defined below, but may also include additional functional or universal elements such as a linker binding region, an adapter, a label etc.

In some examples, a tag or segment thereof may be single-stranded. In some examples, a tag or segment thereof may be double-stranded or partially double-stranded. In some examples, at least one end of a double-stranded tag is a blunt end or an overhang end, including a 5′ or 3′ overhang end. In some examples, a tag or a segment thereof can include a nucleotide sequence that is identical or complementary to any portion of a nucleic acid molecule, amplification primer, etc.

Tags used in methods disclosed herein may be 10 to 1,000 or 50-500 nucleotides or base pairs in length. In some examples, each tag in the set of nucleic acid tags may be between about 30 and about 200 nucleotides in length, or between about 45 and about 90 nucleotides in length. Tag lengths may be adapted depending on the required number and lengths of included functional sequence elements but should not extend lengths that would compromise the tagging procedure (e.g., where tagging is achieved by amplification) or any downstream processing step.

Where tagging is conducted by amplification (i.e., attachment of a tag to a target nucleic acid molecule by fusion PCR), the nucleic acid tag is designed to comprise a sequence region that allows for hybridization with a specific sequence region contained in the target nucleic acid molecule. Such specific binding region may be referred to as a “linker binding region” and be complementary to a linker region present at the 5′ and 3′ regions of the one or more target nucleic acid molecules in the mixture. In a preferred embodiment the linkers and linker binding regions are universal to allow tagging of substantially all nucleic acid molecules in a mixture. In many instances the linker binding region may be between about 10 and about 50 (such as e.g., 15, 20, 25 or 30) nucleotides in length.

Further, the set of nucleic acid tags may comprise a first subset of tags for attachment to the 5′ end of the nucleic acid molecules and a second subset of tags for attachment to the 3′ end of the nucleic acid molecules or vice versa. Tag subsets may be designed and/or produced separately. For example, all nucleic acid tags in a first subset may comprise the same universal sequence elements (such as a linker binding region, a handle, an adapter etc.). Likewise, all nucleic acid tags in a second subset may comprise the same universal sequence elements (such as a linker binding region, a handle, an adapter etc.) which may, however, differ (e.g., in nucleotide sequence) from the corresponding elements of the first subset of tags.

Tagging may also be achieved by blunt-end ligation of nucleic acid tags to both ends of a target nucleic acid molecule in the presence of a ligase (e.g., T4 DNA ligase, Thermo Fisher Scientific). In some instances, tags in the set of nucleic acid tags may be phosphorylated at their 5′ ends for subsequent ligation to a target nucleic acid molecule. Ligation-based tagging may be preferred in certain instances, as tag regions can be designed without overlaps for fusion PCR resulting in shorter tags and thus, reduced synthesis efforts. In case of ligation, the tags are provided as double-stranded molecules.

Each tag in the set of nucleic acid tags further comprises at least one barcode. The term “barcode”, as used herein, refers to a nucleic acid segment or sequence that is specific for a nucleic acid molecule and used to identify this nucleic acid molecule as coming from a particular sample or source or having a desired sequence.

Barcodes may be used to identify individual nucleic acid molecules that result in the generation of specific amplification products after, for example, multiple rounds of amplification (molecule origin tracing). In such instances, barcodes may be used, for example, to determine the effects of “PCR heterogeneity”. Barcodes are connected to individual nucleic acid molecules in the one or more mixtures (e.g., by tagging via amplification or ligation as described above) in a manner such that each nucleic acid molecule (or in a diluted portion thereof) is statistically connected to a different barcode that remains associated with it during subsequent amplification processes. The origin of amplified nucleic acid molecules may then be traced back to starting nucleic acid molecules or certain locations or samples comprising those starting nucleic acid molecules (e.g., a well or compartment). This is typically done by sequencing of nucleic acid molecules present after amplification as further described below.

Barcodes may be of any number of lengths but they will typically be long enough such that they can be readily identified (e.g., by hybridization, by sequencing, etc.) but short enough so that they do not interfere with processes related to the workflows they are used in. For example, a two hundred nucleotide barcode would typically not be used to identify nucleic acid molecules when identification is done by next generation sequencing (“NGS”). This is so because of the limited read lengths of NGS sequencing platforms. In addition to the selected sequencing platform, barcode lengths may also depend on the method used for tagging (amplification or ligation), the presence of other functional elements together determining overall tag length restrictions, the required diversity (i.e., longer barcodes confer higher diversity), which may depend on the number of nucleic acid molecules to be analyzed. Typically barcode regions within a tag may be between about 5 and about 60 nucleotides in length (e.g., from about 7 to about 50, from about 10 to about 50, from about 7 to about 45, from about 7 to about 40, from about 5 to about 35, from about 5 to about 30, from about 5 to about 25, from about 5 to about 15, from about 7 to about 15, from about 10 to about 30 etc. nucleotides in length). In many instances, a barcode may have a length that is sufficient to allow for binding of a barcode-specific primer such as, e.g., between about 10 and about 30 bases (e.g., 10, 12, 15, 18, 20, 25 or 30 bases or between 18 and 25 bases etc.). Barcodes may be further designed to have a melting temperature (Tm) within a certain range to allow for similar or equal annealing efficiency in a PCR amplification reaction, e.g., with barcode primers used for specific amplification of one or more barcoded nucleic acid molecules. In some examples, the Tₘ of all barcodes of a library may fall within a range from about 50 to about 55 °C.

Where barcodes are used to identify desired nucleic acid molecules (e.g., nucleic acid molecules that do not have errors), the nucleotide composition of each barcode within a barcode library may be designed to (i) allow for sufficient distinction between all barcodes even if sequencing errors occur (e.g., where a sequencing-based mutation in a first barcode would result in a sequence that is identical with the sequence of a second barcode), and to (ii) allow for specific amplification of a desired nucleic acid molecule in a mixture carrying an individual barcode (e.g., by using a set of barcode primers, wherein each primer only hybridizes to one specific barcode). In some instances, sufficiently distinct barcodes may be designed using “edit distance” (also referred to as “Levenshtein distance”) or “Hamming distance” parameters. The Levenshtein distance is a string metric for measuring the difference between two sequences. For example, the Levenshtein distance between two barcodes is the minimum number of single nucleotide changes (insertions, deletions or substitutions) required to change one barcode into another barcode. In some instances, a barcode library used for a workflow as disclosed herein may be designed with a Levenshtein distance of an overall minimum of 2, 3, 4, 5, 6, 7, 8, 9 or 10 (i.e., requiring a minimum of 2, 3, 4, 5, 6, 7, 8, 9 or 10 changes to convert into another barcode). The Levenshtein distance may vary between two barcodes of a barcode pair that flank a target nucleic acid molecule. The Levenshtein distance may even vary over the length of the barcode molecules. For example, in a first instance the Levenshtein distance at the 3′end of a barcode may be different than the Levenshtein distance set for the 5′end. In one example, all 20-base barcodes of a barcode library may have an overall Levenshtein distance minimum of 5 with a Levenshtein distance of minimum 2 set for a certain amount of bases in the 3′region of the barcodes (e.g., the first 5-10 bases of the 3′end of each barcode) to allow for a higher diversity at the 3′end which determines primer binding specificity in step (f) of the method as described further below. Thus, in some instances, between about 50% and about 70% of bases at the 5′end may have a higher Levenshtein distance than the remaining 30% to 50% bases at the 3′end. Barcodes may be made and designed in a number of ways. In instances, the plurality of barcodes of the set of nucleic acid tags may have a Levenshtein distance of minimum 2 to 5 and/or have a melting temperature of between 50 °C and 55 °C.

The first and second subset of nucleic acid tags may each comprise between about 100 and about 1,000 different barcodes or between about 200 and about 500 different barcodes. Barcodes may be designed as described above. Combinations of the first and second subset of tags may thus result in between about 10,000 and about 100,000 different barcode combinations. Preferably, the first and second subsets of nucleic acid tags may each comprise between about 10 and about 1,000 different barcodes resulting in between about 10×10=100 and about 1,000×1,000=100,000 different barcode combinations. For purposes of illustration only, a first subset of nucleic acid tags to be attached to the 5′ ends of the nucleic acid molecules may comprise, e.g. 223 defined barcode sequences, whereas a second subset of nucleic acid tags to be attached to the 3′ ends of the nucleic acid molecules may comprise, another 223 defined barcode sequences, such that a combination of both sets results in nearly 50,000 possibilities.

Barcodes may be derived from a barcode library. In some examples, the barcode library may be designed such that each barcode is sufficiently different from another barcode in the library so that the library of barcodes can be used as barcode primers in step (h) of the methods, wherein each barcode primer comprises a barcode-specific binding region that is complementary to one specific barcode in the mixture of tagged nucleic acid molecules.

One exemplary approach to design a barcode library may rely on a method comprising the following steps: (a) designing, in silico a set of candidate barcode sequences having desired sequence properties (e.g., a pre-determined GC content, length, melting temperature, or other properties known to be desirable for primer sequence function); (b) picking, in silico one candidate barcode of the set of candidate barcode sequences and (c) removing, in silico all candidate barcode sequences from the set of candidate barcode sequences that are “too similar” to the picked candidate barcode sequence, where similarity is determined by identifying the number of deletions, insertions or mutations necessary to transform one candidate barcode sequence to another candidate barcode sequence, a parameter known as the “edit distance” or “Levenshtein distance” between two sequences (see, e.g., “Introduction to Algorithms”, Third Edition (2009) by Cormen et al., The MIT Press Cambridge, Massachusetts London, England). Alternatively, “Hamming distance” (as described, e.g., in Bystrykhor “Generalized DNA Barcode Design Based on Hamming Codes”, PLoS One, 2012, Vol. 7, issue 5, e36852) or other appropriate distances may be used to remove barcode sequences of high similarity. The method may further comprise the step d) of iteratively picking further candidate barcode sequences until no candidate sequences of high similarity remain. The resulting pool of picked barcode sequences forms the barcode library with barcodes sufficiently different from each other. In some examples, the candidate barcode picked in step (b) may be chosen randomly or by using a “greedy” approach such that a plurality of barcode libraries with varying barcode numbers may be obtained from which the largest one may be chosen.

In some instances, each nucleic acid molecule within the mixed population of nucleic acid molecules may receive an individual barcode. This may be achieved by using a barcode library with a diversity that exceeds the diversity of nucleic acid fragments in the pool. In other instances, it may not be required to tag each molecule with an individual barcode. For example, where the starting pool of target nucleic acids has a high error rate (e.g., introduced during oligonucleotide synthesis or subsequent assembly steps), the number of different target molecules may exceed the number of barcodes used for tagging the molecules, since the barcode diversity may be limited by a minimum “Levenshtein distance” stringency and/or primer binding requirements as discussed above.

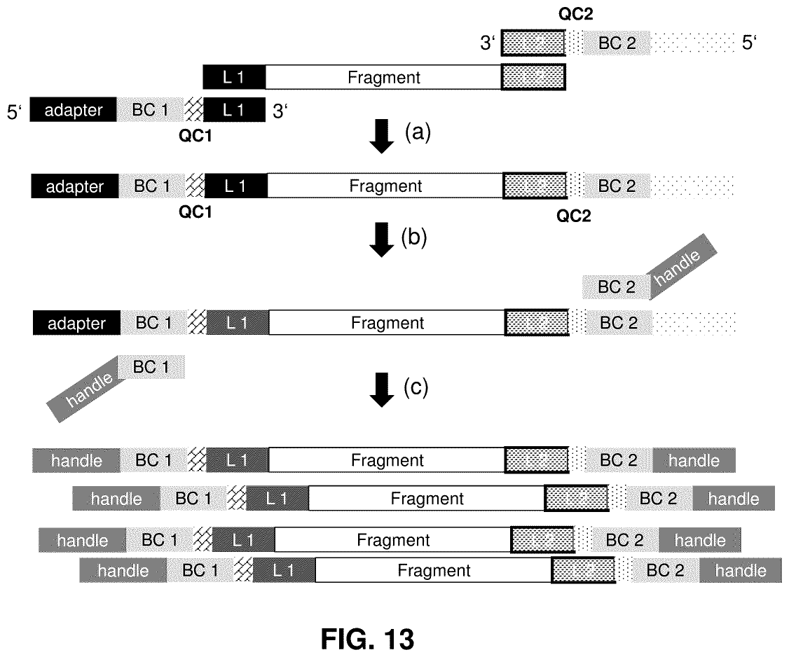

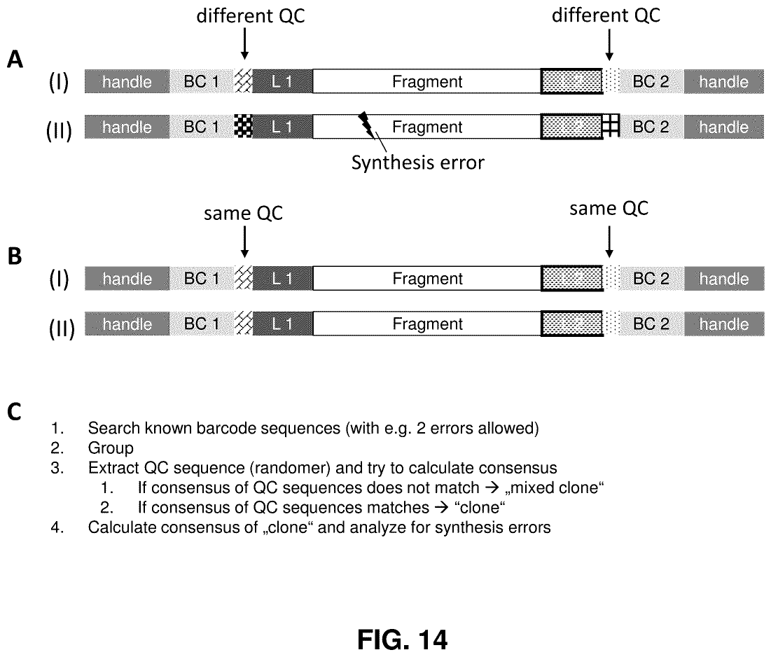

In instances where a limited number of barcodes or barcode sets is used, several nucleic acid molecules in a mixture may be tagged (and then later amplified) with the same barcode sequence combination at their 5′ and 3′ ends. In most cases, a “double usage” would be detected by the downstream sequence analysis. However, when using error-prone NGS, it might be difficult to distinguish two sequence “clones” with the same barcode combination, that differ only by one or few synthesis errors. This could lead to errors that are overlooked, and thus a mixture of correct and incorrect nucleic acid molecules may be selected for subsequent processing or assembly steps resulting in such errors being carried over to the final nucleic acid assembly product. Such “double usage” can be detected and avoided by adding a short random or degenerate sequence to one or both barcodes of a barcode pair as illustrated in FIG. 13, such that this short random sequence (referred to herein as “quality control” or “QC” sequence) is also subsequently amplified in the tagged nucleic acid molecules. If different tagged nucleic acid molecules in a given mixture carry the same barcode combinations after amplification, the QC sequences flanking those nucleic acid molecules at one or both ends can be used to distinguish those nucleic acid molecules from each other as illustrated in FIG. 14.

In some examples, QC segments may comprise a random sequence defined by two or more degenerate positions (e.g., between 2 and 10, or between 4 and 20), meaning that two or more bases can be present at a specific sequence position. In some instances, such QC barcodes can be made in a single synthesis run using, for example, “dirty bottle” synthesis where mixtures are used that result in the incorporation of two or more bases at specific location. Typically, the incorporation of the different bases will be designed to be fully random but concentrations of “building blocks” may be adjusted so that one base is present in a higher or lower ratio than one or more other base. A random sequence of a barcode may also be referred to as quality control or “QC” sequence herein.

In some instances, each barcode of at least one subset of nucleic acid tags is flanked by or comprises a QC region having at least one, at least 2, or between about 3 and about 10 degenerate nucleotide positions, optionally wherein the degenerate nucleotide positions are located at the 3′ end of the barcode as shown in FIG. 13.

Barcodes that are designed individually to have specific sequences may be produced in separate nucleic acid synthesis runs. Like oligonucleotides, barcodes may be synthesized (separately or as part of a tag) by any method disclosed herein such as e.g., on a microchip or array in microscale amounts using standard chemistry, electro- or photochemistry or enzymatic synthesis as disclosed elsewhere herein.

Barcodes may also be concatenated to form a longer, multi-component barcode. For example, where nucleic acid sample mixtures are distributed in multiple vessels (e.g., wells of a 96-well plate), a first barcode may be designed to associate an individual nucleic acid sequence with the respective “source plate”, whereas a second barcode may be designed to associate an individual nucleic acid sequence with a respective “source well”. Both standardized identifiers may be attached to nucleic acid samples in addition to individual barcodes. In this manner, the combination of a small number of (standardized) barcodes can yield high numbers of individual composite barcodes (e.g., a combination of 2×10 separate barcodes results in 100 individual barcodes).

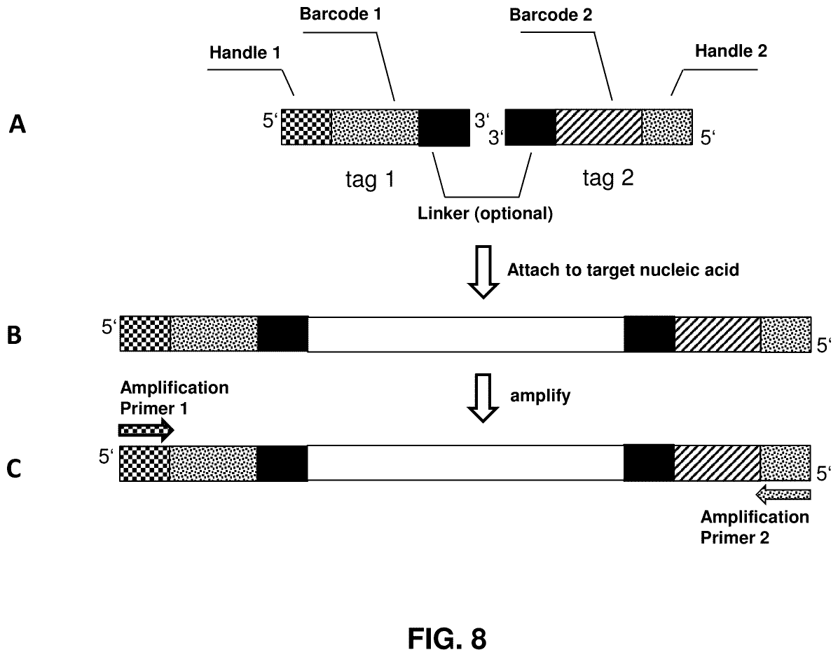

Each tag provided in step (b) of the method may further comprise a handle. The term “handle” as used herein, refers to a segment or portion of a nucleic acid tag that provides an initiation site or universal binding site for a primer. Primers capable of hybridizing to a handle sequence (i.e., having a handle-complementary sequence region) may also be referred to as amplification primers or sequencing primers. A handle may be incorporated into a target nucleic acid molecule as part of a nucleic acid tag and may be attached to the 5′ end, 3′ end or both ends of a tagged nucleic acid molecule, as shown e.g., in FIG. 8. In many instances a 5′ handle and/or a 3′ handle may comprise a “universal” sequence that is the same or substantially the same in every nucleic acid molecule in a mixture to allow for subsequent attachment of a universal adapter. Handles used in methods herein may have various lengths but would typically be between about 5 and about 50, between about 10 and about 30 or between about 15 and about 30 nucleotides in length.

A handle may provide a universal binding site for attaching an “adapter”. As used herein, the term “adapter” refers to a non-target nucleic acid component, which is joined to a target nucleic acid molecule and serves a function in subsequent analysis of the nucleic acid molecule. In many instances, an adapter includes a nucleotide sequence that permits identification, recognition, and/or molecular or biochemical manipulation of the nucleic acid molecule to which the adapter is attached. For example, an adapter may include a sequence which may be used as a primer binding site to read the sequence of the nucleic acid molecule. In another example, an adapter may include a barcode sequence which allows barcoded nucleic acid molecules to be identified. Adapters may also be used, for example, to graft a nucleic acid molecule to a support (e.g., bead, flow cell, bottom of a well or array of reaction sites). In some examples, an adapter can have any length, including fewer than 10 bases in length, or about 10-20 bases in length, or about 20-50 bases in length, or about 50-100 bases in length, or longer. For example, the length of the adapter may depend on the requirements of a certain sequencing platform. An adapter can have any combination of blunt end(s) and/or sticky end(s). In some examples, at least one end of the adapter can be compatible with at least one end of a nucleic acid molecule. In some examples, a compatible end of the adapter can be joined to a compatible end of a nucleic acid molecule. In some examples, the adapter can have a 5′ or 3′ overhang end. In some examples, the adapter can include an internal nick. In some examples, the adapter can have at least one strand that lacks a terminal 5′ phosphate residue. In some examples, the adapter lacking a terminal 5′ phosphate residue can be joined to a nucleic acid molecule to introduce a nick at the junction between the adapter and the nucleic acid fragment. In some examples, the adapter can include an oligo-dA, oligo-dT, oligo-dC, oligo-dG or oligo-U sequences. In some examples, the adapter can include one or more inosine residues. In some examples, the adapter can include at least one scissile linkage. In some examples, the scissile linkage can be susceptible to cleavage or degradation by an enzyme or chemical compound. Optionally, the adapter includes at least one base that is a substrate for a DNA glycosylase. For example, the adapter may include an uracil base that can be excised with uracil N-glycosylase (UNG), upon which the backbone may be cleaved at the abasic site by formamidopyrimidine DNA glycosylase (Fpg). In some examples, the adapter can include at least one phosphorothiolate, phosphorothioate, and/or phosphoramidite linkage. Adapters often include sequencing platform-specific sequences for nucleic acid fragment recognition by the sequencer such as the P5 and P7 sequences that enable library fragments to bind to the flow cells of Illumina platforms. Each NGS instrument provider uses a specific set of adapter sequences for this purpose. Thus, adapter sequences that can be used in the disclosed methods may vary depending on the used sequencing platform. Also, adapters may be incorporated into tags or sequencing primers, depending on the step at which they are to be added to a target nucleic acid. Depending on context, sequencing primers or amplification primers may be used interchangeably herein.

Barcodes and/or sequencing adapters and/or linkers may further be designed to comprise at least one recognition site for a restriction endonuclease and/or a cleavage site for said restriction enzyme. In some examples, a restriction enzyme recognition sequence may be selected from one or more of type I, type II, type Iis, type IIB, type III, type IV restriction enzyme recognition sequences, or recognition sequences having palindromic or non-palindromic recognition sequences. Such cleavage site can be used to remove undesired sequence segments that are not required for downstream processing steps, such as the assembly of a desired nucleic acid molecule. In examples, the cleavage and recognition sites do not overlap. For example, the restriction site may be a type Iis restriction enzyme cleavage site. Alternatively, a restriction enzyme recognition site may be contained in the nucleic acid fragments and respective cleavage sites may be positioned to allow for complete removal of barcode and/or sequencing adapters prior to subsequent assembly with other fragments as laid down below.

In step (c) of the above method where molecules are first diluted and then tagged, the concentration of nucleic acid molecules in each of the one or more mixtures is determined. Alternatively, where molecules are first tagged and then diluted, the concentration of nucleic acid molecules may be determined after the tagging step. Hence, more generally the concentration of nucleic acid molecules may be determined prior to a dilution step. Concentration of nucleic acid molecules can be determined or validated by various means including but not limited to qPCR, digital PCR, optical measurement, spectrophotometry or fluorometric detection (e.g., by using fluorescent markers such as Picogreen, SYBR Green, or other qPCR markers). Exemplary methods for measuring DNA copy number concentration are described in Corbisier et al., Anal Bioanal Chem (2015) 407:1831-1840. In some examples, a photometric UV-Vis DNA quantification instrument for microvolume analysis may be used such as the NanoDrop or NanoDrop 8000 spectrophotometers or the Multiskan SkyHigh Microplate Spectrophotometer or the Varioskan LUX Multimode Reader (Thermo Fisher Scientific Inc.) according to the manufacturer instructions. One way of determining the concentration of nucleic acid molecules in a mixture using a NanoDrop™ 8000 instrument is described in Example 2 below.

In step (d) of the method where molecules are first diluted and then tagged, or in step (e) of the alternative method where molecules are first tagged and then diluted, one or more, preferably two or more first compartments are provided (e.g., at least 2, at least 5, at least 10, at least 50, at least 100, at least 300, etc.). The number of compartments typically varies depending on the number of provided mixtures of nucleic acid molecules but will typically be between 2 and about 1,000, between about 10 and about 500, between about 30 and about 300, or between about 50 and about 200.

The term “compartment” as used herein refers to a separate room, partition or space that allows for a reaction to take place under defined or controlled conditions and/or with defined components. A compartment may have any suitable shape or size or format. In some instances, a compartment may be a well (e.g., a well of a multiwell plate, a microtiter plate or a chip), a cup, a tube, a container, a vial, a vessel, a column, a channel, a chamber, a drop etc. In some instances, the one or more first compartments are selected from wells of a microwell plate, chambers of a cartridge, micro-vessels, or columns.

The one or more first compartments may have a volume of between about 0.1 μl and 1 ml, preferably between about 10 μl and about 200 μl. In some examples, the one or more first compartments are wells of 96-well plates or of 384-well plates.

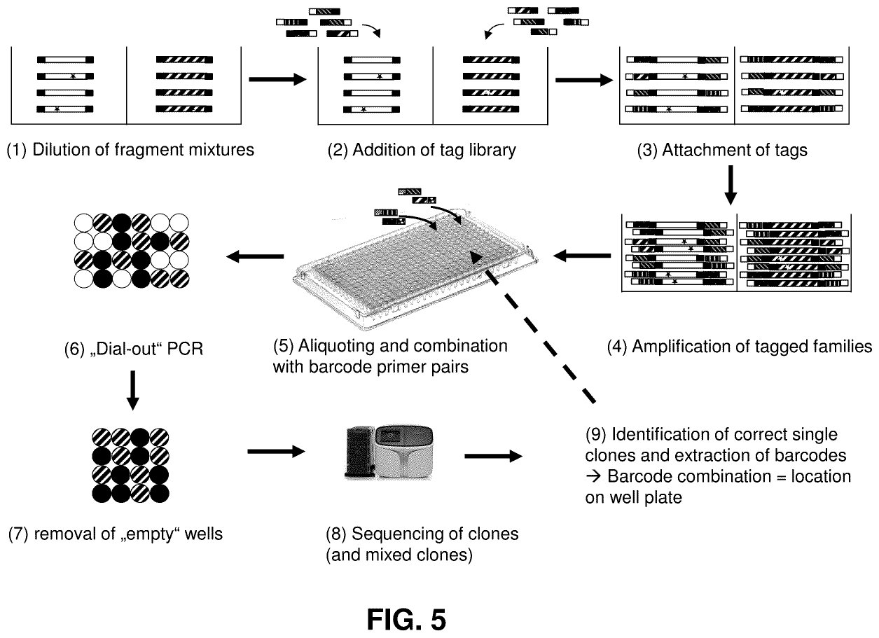

Step (d) of the method where molecules are first diluted and then tagged or step @ of the alternative method where molecules are first tagged and then diluted further comprises diluting each of the one or more mixtures of nucleic acid molecules in a separate first compartment to obtain diluted mixtures of nucleic acid molecules, wherein each diluted mixture comprises a predetermined amount of nucleic acid molecules. Once the concentration of optionally tagged nucleic acid molecules in each mixture has been determined©, a calculated volume of each mixture of nucleic acid molecules may be transferred into each first compartment, e.g., by pipetting or by using acoustic liquid handling devices such as an Echo™ Liquid Handler (Labcyte Inc.). This step is also illustrated by step (1) in FIG. 5.

A suitable dilution may also be obtained by generating serial dilutions of each mixture of nucleic acid molecules and selecting a dilution determined to comprise a desired number of nucleic acid molecules. One way of obtaining dilutions with a predetermined number of nucleic acid molecules is set out in Example 2 below.

In some examples, to obtain a predetermined amount of nucleic acid molecules, each mixture of nucleic acid molecules may be diluted by between about 1:10,000 and about 1:10,000,000,000. Thus, in some examples, serial dilutions of between about 10⁻⁴ and about 10⁻¹⁰ may be generated for each mixture to arrive at the predetermined number of molecules.

In some examples the predetermined amount of nucleic acid molecules in each first compartment may comprise between about 20 and about 1,000,000, preferably between about 20,000 and about 80,000 and more preferably between about 30,000 and about 50,000 molecules. In a preferred embodiment, the predetermined amount of nucleic acid molecules in each first compartment represents the number of barcode combinations of the first set of nucleic acid tags.

In step (e) of the method where molecules are first diluted and then tagged, the diluted mixtures of nucleic acid molecules in one or more of the first compartments are contacted with the set of nucleic acid tags. The contacting may comprise adding a portion of the first set of nucleic acid tags to each one of the one or more first compartments comprising a diluted mixture of nucleic acid molecules. A tag is then attached to both ends of substantially each nucleic acid molecule in the one or more first compartments to obtain tagged nucleic acid molecules having at least a barcode region and a handle. This step is also illustrated by (2) and (3) in FIG. 5.

Alternatively, in step (c) of the method where molecules are first tagged and then diluted, the one or more mixtures of nucleic acid molecules are contacted with the set of nucleic acid tags and a tag is attached to both ends of substantially each nucleic acid molecule to obtain tagged nucleic acid molecules having a barcode region and a handle or adapter region at both ends. The concentration of the tagged nucleic acid molecules in the one or more mixtures may then be determined before each of the one or more mixtures will be diluted in a separate first compartment to obtain diluted mixtures of tagged nucleic acid molecules, each diluted mixture comprising a predetermined amount of tagged nucleic acid molecules.

Tagging “substantially each nucleic acid molecule” means that a sufficient number of tag molecules is added to the mixtures of nucleic acid molecules such that statistically each nucleic acid molecule receives a tag. However, it cannot be completely excluded that few individual molecules may remain untagged.

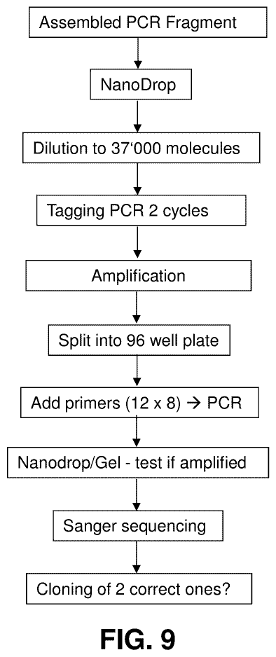

In some instances, the nucleic acid tags may further comprise an adapter (e.g., a universal sequencing adapter), such that the adapter would also be attached to the nucleic acid molecules during the tagging step as described e.g. in FIG. 9. In other instances, adapters may also be attached as part of the amplification primers (see step (f) below). In many instances the attaching may be performed by hybridizing the linker binding sequence of the nucleic acid tags to the linker regions within the target nucleic acid molecules and performing a PCR reaction. For PCR-based tagging, the number of PCR cycles may be limited to not more than five, preferably not more than two cycles to maintain the ratio of the input diversity of nucleic acid molecules. Alternatively, the attaching may be performed by ligation in the presence of a ligase as described above.

In subsequent optional step (f) one or more pairs of amplification primers are provided. The amplification primers are designed to hybridize to both ends of the tagged nucleic acid molecules (e.g., the handle region of a tag). In some instances, both primers of the pair may further comprise an adapter at the 5′ end as illustrated in FIG. 8. In other instances, the adapter may already be present by attachment as part of the nucleic acid tag (e.g. as shown in (3) of FIG. 5). As discussed above, adapters are typically required for downstream sample preparation for sequencing. Thus, an amplification primer containing a sequencing adapter may also serve as a sequencing primer.

The tagged nucleic acid molecules are then optionally amplified by PCR in the one or more first compartments in the presence of the one or more pairs of amplification primers. The amplification step results in families of tagged nucleic acid molecules, wherein all nucleic acid molecules in a family share the same combination of barcodes (and optionally QC sequences). This step is also illustrated by step (4) in FIG. 5.

In contrast to the tagging step, the amplification step typically includes more than 2 PCR cycles to generate sufficient amounts of nucleic acid molecules for subsequent analysis. In some examples, the number of PCR cycles comprises between about 15 and about 30, or between about 20 and about 30.

To limit the amount of errors introduced during PCR-based tagging (step (e)) and/or amplification (step (f)), a high fidelity polymerase with 3′-5′ exonuclease proofreading activity may be used as disclosed above.

In optional step (g) the amplified nucleic acid molecules may further be purified. The purification step may be useful to remove reagents of the PCR step such as dNTPs, primers, polymerase and the like. Purification of clonal amplicons can be achieved by various means including, for example, bead-based extraction (as described e.g., in Boom et al., J Clin Microbiol. (1990) or in DeAngelis et al., Nucleic Acids Res. (1995) 23 (22): 4742-3), by gel extraction, column purification or precipitation. Multiple methods and kits for purifying nucleic acids are available including the following: Thermo Scientific™ GeneJET™ PCR Purification Kit (Thermo Fisher Scientific, Cat. No. K0701), QIAquick™ PCR Purification Kit (Qiagen, Cat. No. 28104), NucleoSpin™ Gel and PCR Clean-up kit (Macherey-Nagel, Cat. No. 740609.10). Invitrogen™ PureLink™ PCR Micro Kit (Thermo Fisher Scientific, Cat. No. K310050), MinElute™ PCR Purification Kit (Qiagen, Cat. No. 28004), Invitrogen™ ChargeSwitch™ PCR Clean-Up Kit (Thermo Fisher Scientific, Cat. No. CS12000), MagAttract™ PowerClean DNA Kit (Qiagen, Cat. No. 27900-4-KF), Agencourt AMPure XP kit (Beckman Coulter, Cat. No. A63880).

Subsequent step (h) provides a set of barcode primers, wherein each barcode primer of the set comprises a barcode-specific region designed to hybridize to a specific barcode in the set of nucleic acid tags, or in other words: designed to hybridize to a specific barcode in the tagged (and amplified) nucleic acid molecules. Typically barcode primers are designed for efficient and specific hybridization with the specific target region and may be between about 15 and about 50 nucleotides in length. Further, the number of barcode primers in the set of barcode primers typically equals the number of barcodes in the first set of nucleic acid tags. For example, the set of barcode primers may comprise between about 100 and about 10,000 or between about 200 and about 2,000 different barcode-specific regions.

Further, the set of barcode primers may comprise a first subset of barcode primers designed to hybridize to barcodes located at the 5′ ends of the (tagged) nucleic acid molecules and a second subset of barcode primers designed to hybridize to the barcodes located at the 3′ ends of the (tagged) nucleic acid molecules. In some examples, the first and second subset of barcode primers may each comprise between about 10 and about 5,000 or between about 100 and about 1,000 different barcode-specific regions. Generally, the set of barcode primers may comprise the same diversity of different barcode sequences as provided in the tagged nucleic acid molecules.

In some examples, each barcode primer in the set may further comprise a label. The label may be positioned at the 5′ end of a barcode primer. The label may be used for subsequent processing steps, such as amplicon capture/enrichment during sample preparation prior to sequencing. In one example, the label may be biotin. For example, biotin-labelled amplicons may be captured and enriched using magnetic Streptavidin-coated beads such as e.g., Dynabeads™ MyOne™ Streptavidin magnetic beads (Thermo Fisher Scientific Inc.) In some instances, the label may comprise a sequence region designed to hybridize with a probe.

In step (i) one or more second compartments are provided. In many instances, more than one (i.e., two or more) second compartments will be provided. Typically, the number of second compartments may vary between about 5 and about 5,000, such as e.g., between 100 and 1,000. In many instances the number of second compartments may be less than or equal to the number of first compartments.

Then, at least a portion of each of the amplified and optionally purified tagged nucleic acid molecules is contacted with a defined pair of the set of barcode primers in such second compartment(s) followed by an amplification reaction as illustrated by step (5) in FIG. 5.

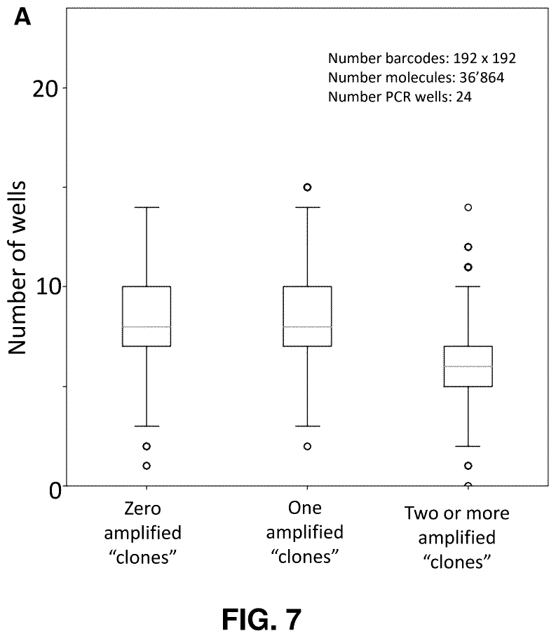

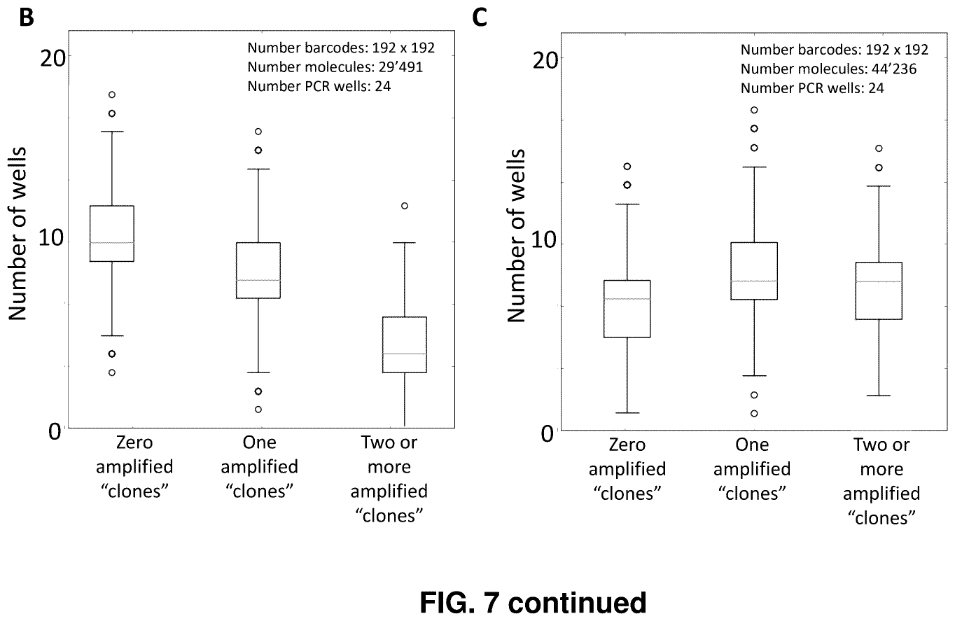

As a result of the amplification reactions the one or more (preferably two or more) second compartments may comprise: (i) one specific amplification product, (ii) no specific amplification product, and/or (iii) two or more specific amplification products, as illustrated in (6) of FIG. 5. In this context “one specific amplification product” means that all amplified nucleic acid molecules are derived from a single/the same template nucleic acid molecule or have substantially the same sequence (not taking amplification errors into account). This is expected when the provided barcode primer pair in a given second compartment specifically binds to (and amplifies) one target nucleic acid that comprises the complementary barcode combination. Where two or more nucleic acid molecules with the matching barcode combination are present in a second compartment, “two or more specific amplification products” are likely to be obtained in that compartment because the respective barcode primer pair binds to (and amplifies) all targets that contain the matching barcode combination. However, in some second compartments no amplification product may be present. This will be the case if none of the nucleic acid molecules present in said second compartment comprise a matching barcode combination for the provided barcode primer pair. As described in Example 1, the number and ratio of second compartments having one specific, multiple or no specific amplification products will be substantially determined by the initial dilution/concentration of nucleic acid fragments in step (d). In that example providing 223 5′ and 223 3′ barcodes (resulting in 49,729 different barcode combinations) a number of 50,000 copies per mixture of nucleic acid molecules resulted in similar distribution of second compartments having one specific amplification product (corresponding to “one amplified clone” in FIG. 7A), multiple specific amplification products (corresponding to “two or more amplified clones” in FIG. 7A), or no specific amplification product (corresponding to “zero amplified clones” in FIG. 7A), respectively. In contracts, if a lower copy number of 20,000 molecules was used, the majority of second compartments did not have any specific amplified clones (see FIG. 7B), whereas a setting with a higher copy number of 80,000 molecules resulted in a majority of second compartments with two or more amplified clones (FIG. 7C). It is thus important to determine the optimal number of starting nucleic acid molecules to obtain a sufficient number of second compartments that will only contain one specific amplification product, while reducing the number of second compartments with two or more amplification products.

In some instances, each second compartment may be preloaded with a different pair of the set of barcode primers before at least a portion of the amplified and optionally purified tagged nucleic acid molecules are added to each second compartment. In other instances, each second compartment may be preloaded with at least a portion of the amplified and optionally purified tagged nucleic acid molecules before a different pair of the set of barcode primers is added to each second compartment. Primer and template concentration may be chosen according to standard PCR protocols.

Further, each second compartment may be provided with (i) the same 5′ barcode primer and a different 3′ barcode primer, (ii) the same 3′ barcode primer and a different 5′ barcode primer, or (iii) a different 5′ primer and a different 3′ primer. If (i) or (ii) are employed, the same barcode primer can be reused for multiple samples, thereby reducing the overall number of barcodes needed. For example, if sequences in a mixture are known to be very different, one could assign either the same 5′ barcode primer or the same 3′ barcode primers to multiple compartments for subsequent retrieval, as sequences could be easily deconvoluted before calculating a consensus.

In step (i), any compartment more generally described above may be used as second compartment. Thus, the one or more second compartments may be selected from wells of a multiwell plate or a cartridge, chambers, vessels, columns or droplets. In some examples the one or more second compartments are wells of a 384-well plate or a 1536-well plate, optionally wherein the plates are configured for use with an acoustic liquid handling device.

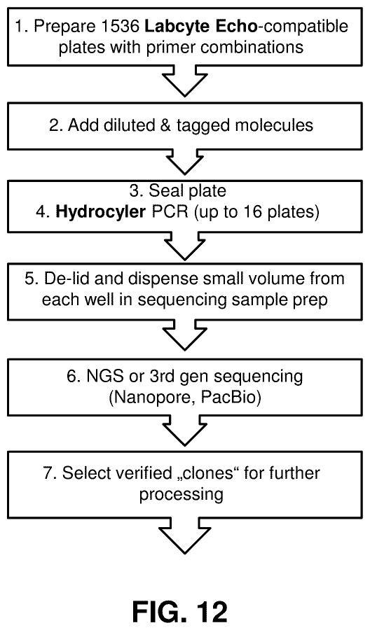

While sequencing can be done in bulk (many parallel reactions in one sequencing run), the PCR amplification step needs to be performed in separated wells, as the final product cannot be mixed with other “clones”. Based on a typical 1 kB nucleic acid fragment with a rather high error rate of 1/800 bp-1/1000 bp, simulations suggest that one would need about 64 PCR reactions to find a correct “clone” in 99% of all cases. If for example typical order volumes of nucleic acid fragments are considered, this can easily lead up to 10'000s of wells, which is not efficient in 96- or 384-well formats. Although efficiency can be increased by switching to a 1536-well plate format, commercial 1536-well plates are either good to handle but not compatible with most PCR-cyclers (such as 1536-well plates used in Echo® Acoustic Liquid Handler, Labcyte Inc.), or are difficult to handle but PCR cycler-compatible (e.g., plates for the Lightcycler®, Roche). To overcome this deficiency, the amplification step may be performed using a 1534-well plate compatible with an acoustic liquid handler (e.g., Labcyte Echo® or mosquito® HTS Nanolitre Liquid Handler by SPT Labtech) in combination with a Hydrocycler (LGC, Biosearch Technologies). The Hydrocycler can process 16×1534 well plates in the same time as a regular PCR cycler, thus providing increased throughput (see https://www.douglasscientific.com/content/pdfs/Hydrocycler2.pdf?y=161223.02). The method thus includes conducting step (i) in a Hydrocycler which embodiment is further described in FIG. 12.

An additional advantage of using acoustic liquid handler-compatible plates is that, after the amplification step, small droplets of nucleic acid material can be acoustically ejected into the sequencing preparation reaction vessel without any need for tips, or risk of contamination. Also, after correct clones (and their position in a first compartment) have been identified by sequencing as set out in step (1) below, the required sample volume can be ejected with the acoustic liquid handler and further processed (e.g., cloned into plasmid, used in higher-order assembly, directly used for transient protein expression etc., as further described below).

The method may further comprise a step (j) of identifying second compartments (ii) having no specific amplification product and/or second compartments (iii) having two or more specific amplification products and excluding such second compartments from subsequent steps. This may be useful to reduce the number of samples in downstream analysis. To identify second compartments having no specific amplification product, the concentration of nucleic acid molecules in each second compartment can be determined, using any of the techniques described above such as qPCR, optical measurement, spectrophotometry or fluorometric detection. For example, PicoGreen™ ds DNA assay (Thermo Fisher Scientific) may be used to distinguish a “real” amplification product from the (non-amplified) input nucleic acid molecules that are only present at very low amounts. Alternatively, second compartments that do not have specific amplification products may also be identified by gel analysis or capillary electrophoresis, e.g., where amplification products differ by length.

Following selection of second compartments containing desired amplification products for subsequent analysis, the method may further comprise (k) optionally pooling at least a portion of the amplification products from two or more of the second compartments. The pooling may comprise combining at least a portion of the amplification products from multiple second compartments. In cases where no sequencing adapters are present in the amplification products, required tags or adapters for subsequent sequencing may be attached to the pooled nucleic acid molecules. Thus, the combined amplification products may be contacted with a further set of nucleic acid tags to attach the further set of nucleic acid tags to both ends of substantially each amplification product using tags and techniques described above.

In step (1) amplification products of selected compartments are subject to sequencing to obtain sequence data as shown in step (8) of FIG. 5. Since the method is agnostic for every sequencing technique, the sequencing may comprise sequencing by synthesis (SBS), sequencing by ligation, Sanger sequencing, single molecule sequencing, third generation sequencing etc. In some instances, the amplification products may need to be fragmented prior to sequencing. In such case sequencing adapters or other sequence platform specific tags or moieties may need to be attached to the fragmented amplification products.

A number of nucleic acid sequencing methods are known in the art and include Maxam-Gilbert sequencing, chain-termination sequencing (e.g., Sanger sequencing), pyrosequencing, sequencing by synthesis and sequencing by ligation. Methods of the disclosure may use any type of sequencing platform suitable for the intended purpose, including any next-generation sequencing platform such as: sequencing by oligonucleotide probe ligation and detection (e.g., SOLID™ from Thermo Fisher Scientific, (see e.g., PCT Publication No. WO 2006/084131), probe-anchor ligation sequencing (e.g., COMPLETE GENOMICS™ or POLONATOR™), sequencing-by-synthesis (e.g., GENOME ANALYZER™ and HISEQ™, from Illumina), pyrophosphate sequencing (e.g., Genome Sequencer FLX from 454 Life Sciences), single molecule sequencing platforms (e.g., HELISCOPE™ from HELICOS™), and ion-sensitive sequencing (e.g., Personal Genome Machine, Proton and Ion S5 from ION TORRENT™ Systems, Inc.) as set out, e.g., in PCT Publication WO 2012/044847 or U.S. Pat. No. 7,948,015. For an overview, see, e.g., Mardis E. R., “Next-Generation Sequencing Platforms”, Annu. Rev. Anal. Chem. 6:287-303 (2013).

Although Sanger sequencing is highly accurate it may not be suitable for high throughput settings as a rather expensive technology. Alternatively, despite its low error rate and high throughput, the short-read length of Illumina or Ion Torrent platforms may restrict the use of such SBS technologies for longer nucleic acid fragments.

Thus, in some instances, sequencing techniques often referred to as third generation sequencing including nanopore sequencing offered by Oxford Nanopore Technologies (ONT) or PacBio single molecule real-time (SMRT) sequencing (such as a PacBio circular consensus sequencer) may be used, both of which allow for sequencing of long-read amplicons.

Nonlimiting examples of third-generation sequencers are PacBio RS II. PacBio RS II (Pacific Biosciences, Menlo Park, CA, USA). PacBio RS II is able to sequence single DNA molecules in real-time without means of amplification such as PCR, enabling direct observation of DNA synthesis by DNA polymerase. SMRT technology offers four major advantages compared to first- and second-generation platforms: (1) long read lengths (half of data in reads >20 kb and maximum read length>60 kb, for example read lengths between 10 kb on the low end of the range and IM kb, 500,000 kb, 250,000 kb, 100,000 kb, 90 kb, 80 kb, 75 kb, and 60 kb on the high end of the range, or between 20 kb on the low end of the range and IM kb, 500,000 kb, 250,000 kb, 100,000 kb, 90 kb, 80 kb, 75 kb, and 60 kb on the high end of the range, (2) high consensus accuracy (for example >99.999% at 30× in coverage depth, free of systematic elTors), (3) low degree of bias (even or relatively even coverage across G+C content), and (4) simultaneous epigenetic characterization (direct detection of DNA base modifications at one-base resolution). These advantages enable resolution and analysis of hard-to-sequence regions in complex genomes.

In some embodiments, a sequencing step according to methods of the disclosure can include nucleic acid molecules having between 3,000 and 50,000 bp or between 50,000 and 100,000 bp in length and can include sequencing 2,000, 1,000, 800, 600, 500, 400, 300, 200, 150,125, 100, 90, 80, 75, 70, 65, 60, 55, 50, 45, 40. 35, 30, 25, 20, 15, JO, 9, 8, 7, 6, 5. 4, 3, or 2 or fewer nucleic acid molecules.

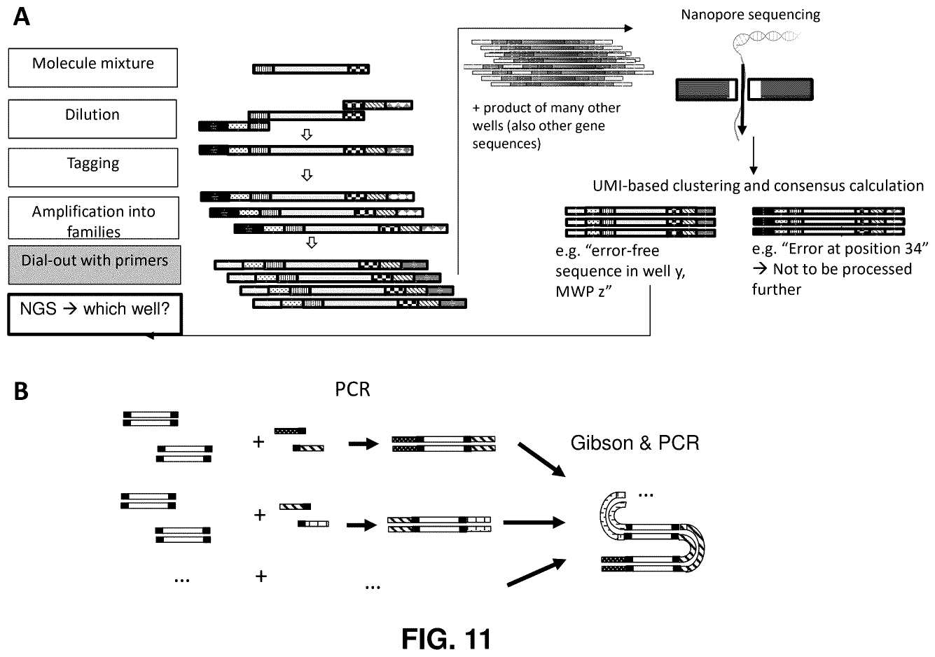

Although these platforms are more error-prone it has been shown that that accurate sequence information from long-read 3rd generation sequencing platforms can be obtained by using UMIs and calculating consensus sequences as described e.g., in Karst et al.; Nature Methods Vol 18 (2021), 165-169 or in Zurek et al, Nature Communications Vol 11, 6023 (2020). Thus, the present methods include embodiments where barcode sequences used for UMI cloning as described above are used for error correction in third generation sequencing. In contrast to the methods cited above, however, the amplification of the UMIs (i.e. specific barcodes) may take place at different steps of the present method, in particular if pre-designed barcodes of known sequence with a good Levenshtein distance as described above will be used. An exemplary UMI cloning workflow using third generation sequencing is described in FIG. 11A.

Further, it has been found that nanopore sequencing of short nucleic acid fragments (e.g., having lengths below ~200 bp) may result in significantly lower sequencing throughput due to inefficient pore utilization. In such instances, short amplification products may be further concatenated to generate longer template nucleic acids to ensure efficient throughput in sequencing as more data can be collected by sequencing one molecule.

In one example, Gibson Assembly® may be used to concatenate short nucleic acid amplicons into long concatemers as shown in FIG. 11B. Gibson Assembly® is an isothermal and single reaction exonuclease-based assembly method first described by Gibson et al., Nat. Methods 6, 343 (2009). Generation of long concatemers (i.e., covalently linked DNA molecules) is based on the presence of overlapping sequences at the ends of the amplicons (hereinafter “overhangs”) that that may be added by providing additional flanking sequences (e.g., appending overhang sequences to both ends of the nucleic acid fragments by PCR using suitable amplification primers). In some examples, suitable overhang sequences may be between about 15 and about 50, between about 20 and about 40 bp long and may have a GC-content of between about 35 and about 60%, preferably between about 30 and about 50%, such as e.g., 40% which has been reported to be optimal for Gibson assembly reactions (Casini, et al., Nucleic Acids Res. 42, e7 (2013)). Overhangs may also be designed using suitable optimization algorithms taking additional sequence parameters into account such as avoidance of self-binding/secondary structures, n-mer nucleotide repeats and cross-talk between orthogonal overhangs etc. Following PCR reactions with primers comprising the respective overhangs, amplicons carrying the flanking overhangs are then mixed at equimolar concentrations and incubated with a Gibson Assembly® master mix (e.g., Thermo Fisher Scientific, NEB) according to the manufacturer's instructions. Alternatively, other exonuclease-based assembly kits or methods may be used such as e.g., the NEBuilder® Assembly tool provided by NEB. In contrast to traditional uses of Gibson Assembly® where fragments are assembled in a specified order, concatenation in this approach occurs in a random fashion, wherein nucleic acid fragments are subsequently added to both ends of the growing chain resulting in vanishing of input monomers over time as concatemers of higher order will accumulate. Gibson Assembly products may be further purified (e.g., using gel-purification) prior to sequencing.

In another example, overlap extension PCR may be used to generate concatemers of multiple amplicons wherein overlapping sequences between fragments are generated by using primers containing 5′ overhangs complementary to the molecules they are to be joined to. The terminal primers may then be used to amplify the full-length of the fused molecules.

Once the sequencing has been performed using any of the methods outlined above the obtained sequence data are analysed in step (m). The analysis comprises obtaining the combinations of barcodes flanking the one or more nucleic acid molecules having a desired sequence. In some instances the combinations of barcodes are obtained by conducting the following steps: (a) aligning sequence reads of the one or more nucleic acid molecules having the same barcode combinations and obtaining one or more consensus sequence of the one or more nucleic acid molecules from the aligned sequence reads, (b) comparing the one or more obtained consensus sequence to the desired sequence of the one or more nucleic acid molecules and selecting the sequences of the one or more nucleic acid molecules having a desired sequence, and (c) determining the sequences of the barcodes flanking the desired nucleic acid molecule.

In one more specific example the obtained sequencing reads may be processed according to the following method: In a first step, the sequencing reads may be grouped according to one or more individual barcodes flanking the fragments. For example, where a fragment is flanked by a barcode pair, all reads having the same barcode pair are grouped into the same “family”, wherein a family represents all nucleic acid molecules that have been amplified from fragments tagged with the same barcode combination. Alternatively, in cases were more than two barcodes are present the reads may be grouped according to three or more barcodes or two or more barcode pairs. When two molecules are amplified with the same barcode combination (which cannot be avoided as exemplified in FIG. 7), it might be difficult to identify a synthesis error, depending on the sequencing technology and its quality. In such instances it may be useful to provide tags with quality control (“QC”) sequences as described above. Where QC sequences are present, reads may first be grouped according, the barcodes and the QC sequences may then be used to identify whether nucleic acid families with identical barcode sets comprise mixed clones. As illustrated in FIG. 14, sequencing data of mixed nucleic acid molecules may be processed by searching and grouping (known) barcode pairs. If present, the short random (and thus abundant) QC sequences may then be extracted from the grouped reads to calculate a QC consensus sequence. If a consensus of QC sequences can be obtained, the clone can be identified as a single clone. On the other hand, if no QC consensus can be calculated, the clone can be identified as mixed clone. Finally, the consensus of identified (single) clone sequences can be calculated and matched with the known target nucleic acid sequence to obtain those sequences that are error-free.

To reduce data complexity, those reads that do not comprise the predetermined set of barcodes and are therefore considered invalid may be discarded. An optional further step may seek to verify the presence of terminal linker regions (e.g., 5′ and 3′ linker regions comprising a restriction endonuclease cleavage site, a homologous region or other functional element for downstream processing) and reads not comprising those linker regions or functional elements may be discarded. All remaining reads belonging to a family are then aligned to generate a consensus sequence from the multiple fragment reads. The consensus for each nucleotide position may be defined based on a predetermined threshold of base calls for base identity. For example, a 70% or 80% or 90% threshold requires at least 70% or at least 80% or at least 90% of all bases at a given nucleotide position to be identical throughout the aligned reads. Those positions that deviate from the consensus (most likely representing “sequencing errors”) may then be corrected in silico. For example, where 80% of reads have an “A” at a defined nucleotide position of the alignment, an individual read indicating a C, T, G or a “gap” (often indicated by a dash in the sequence alignment) at this position may be corrected to “A”. The consensus computation and correction may then be reiterated several times (e.g., up to four times) along the alignment to arrive at a preliminary consensus sequence for each family.

In some instances, the preset threshold (e.g., at least 80%) may not be reached by a single base at certain positions-even after multiple iterations. Therefore, the following strategy may be applied to arrive at a final consensus: When the percentage of the most frequent base at a position is below the preset threshold, and the “gap dash” is among the two most frequent bases, then the percentage of the most frequent base and the percentage of positions with a gap dash are added. If the combined percentages reach the preset threshold and if there is an acceptable small number of such positions, (e.g., 1 or 2 per consensus sequence), they can be marked in the final consensus sequence (e.g., by lower case letters representing the most frequent base or other symbols) wherein such mark indicates either “no base” or the “most frequent base” given by the consensus. Thus, in a case where 2 such positions would be present in a final consensus sequence, the consensus describes a set of up to four different sub-fragment sequences. A consensus sequence that would still comprise undefined positions after the aforementioned correction maybe discarded by marking the associated barcode combination as invalid. One or more of the obtained consensus sequences may then be compared to a database of reference sequences. If there is a 100% match against one of the reference sequences, the amplification product is identified as error-free fragment and can be retrieved based on its individual barcode signature.

In subsequent step (n) one or more of the second compartments comprising one specific amplification product are identified to thereby locate the one or more nucleic acid molecules having the desired sequence. The identifying comprises determining the one or more second compartments comprising the combinations of barcodes that flank the desired nucleic acid molecule (see also step (9) in FIG. 5). In some instances, each barcode pair may be contained in a list assigning a respective well position. Alternatively, each plate-well or compartment may be identifiable based on a computer barcode using respective scanning means.

One or more of the amplification products may then be retrieved from the identified one or more second compartments, which may be achieved by various means including pipetting, ejecting or transferring at least a portion of the amplification products from said one or more second compartments. Alternatively, desired amplification products may also be retrieved by hybridizing the amplification products from said one or more second compartments to a probe, optionally wherein said probe may be immobilized on a solid support (e.g. magnetic beads). Suitable probes may comprise sequence regions complementary to the specific barcodes of the desired nucleic acid fragments.

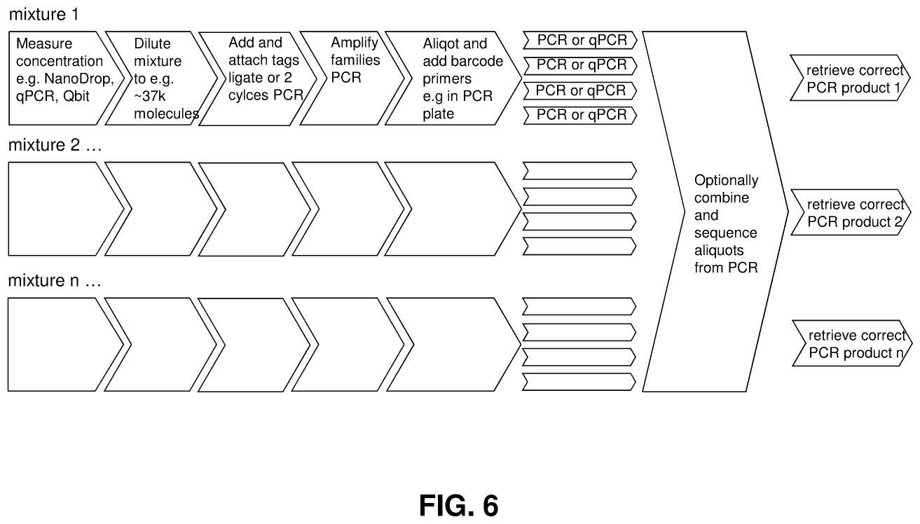

A summary of the workflow starting from 1 to n mixtures of nucleic acid molecules and resulting in 1 to n specific amplification products that can be retrieved for subsequent processing is shown in FIG. 6 for illustration purposes.

### Assembly of Sequence-Verified Fragments

In some instances, the retrieved amplification products comprising desired nucleic acid sequences may be subject to further processing. Such further processing may comprise higher order assembly of two or more of the retrieved amplification products. For example, depending on the length of a desired final nucleic acid molecule, two or more of the sequence-verified amplification products may be further assembled into longer nucleic acids. Therefore, the method may further comprise combining two or more of the identified nucleic acid fragments and assembling the combined fragments into longer fragments to generate the desired nucleic acid molecule. Assembly techniques may vary and may comprise any of the assembly methods known in the art including but not limited to restriction enzyme cleavage and ligation, fusion PCR, homologous recombination or exonuclease-based assembly many of which are described in PCT Publication WO 2006/044956 A1.

Where amplification-based errors are to be avoided, a non-PCR assembly method may be used including e.g., restriction enzyme-based assembly methods. In some instances, the two or more amplification products or nucleic acid fragments selected for further assembly may still comprise flanking regions (such as universal primer binding sites, barcodes, adapters etc.) including one or more restriction enzyme cleavage sites as described above. One subclass of restriction enzymes are restriction endonucleases of type IIs, which cleave a nucleic acid at a fixed position outside their recognition sequence. For example, nucleic acid amplification products having type Iis restriction enzyme cleavage sites incorporated into the flanking linker regions may be treated (either prior to or after combination) with one or more type Iis restriction endonucleases to remove the flanking tags from the 5′ and 3′ ends. Type Iis restriction enzyme recognition sites and type Iis restriction enzymes that are useful in the present cloning and assembly methods include, but are not limited to, AarI, BsaI, BbsI, BbvII, BsmAI, BspMI, Eco31I, BsmBI, BaeI, FokI, HgaI, MlyI, SfaNI and Sth132I. Type Iis restriction site mediated assembly methods may be used to assemble multiple fragments simultaneously (e.g., two, three, five, eight, ten, etc.) when larger constructs are desired (e.g., 5 to 100 kilobases), wherein at the same time any undesired flanking regions are removed. This cloning system also referred to as “Golden Gate cloning” is described in Engler et al. PloS ONE, Vol. 3 (11) e3647 (2008) and Weber et al., PloS One, Vol. 6 (2) e16765 (2011), and is set out in various forms in U.S Patent Publication No. 2010/0291633 A1, PCT Publication WO 2010/040531, U.S. Publication 2008/0287320, in Kotera and Nagai, J. Biotechnology 137, 1-7 (2008) or in U.S. Publication 2009/0155858A1.

Type Iis restriction enzyme based cleavage can generally be used to remove any sequence element (e.g. flanking elements including tags, adapters, linkers, handles, primer binding sites, barcodes and/or UMIs) that should not be included or be part of a desired target nucleic acid molecule (e.g., an assembly product or gene) and such sequence removal can be combined with assembling longer nucleic acid molecules by generating single-stranded overlaps for subsequent annealing of nucleic acid fragments. Alternatively, any undesired sequence elements may also be removed by other means.

For example, where flanking elements are included by amplification (e.g., during a tagging step) such amplification may be performed with uracil-containing primers such that the amplification products contain uracil. The amplification products can then be incubated with a uracil DNA glycosylase (UDG) and DNA glycosylase-lyase Endonuclease VIII such that the uracil bases are removed from the amplification products (e.g., following sequence verification/retrieval of a correct nucleic acid molecule). In some instances, multiple uracils may be included in the primers such that the resulting segments after the uracil bases are removed are short enough that the segments melt off, leaving a single-stranded overhang. The single-stranded overhang can then be truncated using methods known in the art, for example, using the Klenow fragment. Thus, in some examples the removing adapters and/or barcodes or other flanking sequence elements can include a uracil-base removing step including a uracil DNA 67lycolsylase (UDG) and DNA glycosylase-lyase Endonuclease VIII, and a removing a single-stranded overlap step including a nuclease, for example, the Klenow·fagment. A similar method can be used with deoxyinosine-containing primers and endonuclease V. In other embodiments unwanted sequence elements may be removed using incorporated photocleavable nucleotides. Photocleavable nucleotides include, for example, photocleavable fluorescent nucleotides and photocleavable biotinylated nucleotides. See, e.g., Li et al, PNAS, 2003, 100:414-419; Luo et al, Methods Enzymol, 2014, 549:115-131. A skilled person understands how to use any of the methods in the art to remove undesired sequence elements from target nucleic acid molecules compatible with the methods disclosed herein.

In other examples nucleic acid molecules may be assembled into a target vector using a method as described in Yang et al., Nucleic Acids Research 21:1889-1893 (1993) and U.S. Pat. No. 5,580,759. In the process described in Yang et al., a linear vector is mixed with double-stranded nucleic acid molecules which share sequence homology at the termini. An enzyme with exonuclease activity (i.e., T4 DNA polymerase, T5 exonuclease, T7 exonuclease, etc.) is added which generates single-stranded overhangs of all termini present in the mixture. The nucleic acid molecules having single stranded overhangs are then annealed and incubated with a DNA polymerase and deoxynucleotide triphosphates under condition which allow for the filling in of single-stranded gaps. Nicks in the resulting nucleic acid molecules may be repaired by introduction of the molecule into a cell or by the addition of ligase. Of course, depending on the application and workflow, the vector may be omitted. Further, the resulting nucleic acid molecules, or sub-portions thereof, may be amplified by polymerase chain reaction.

A method for the isothermal assembly of nucleic acid molecules is set out in U.S. Patent Publication No. 2012/0053087. In one aspect of this method, nucleic acid molecules for assembly are contacted with a thermolabile protein with exonuclease activity (e.g., T5 polymerase) and optionally, a thermostable polymerase, and/or a thermostable ligase under conditions where the exonuclease activity decreases with time (e.g., 50 °C). The exonuclease “chews back” one strand of the nucleic acid molecules and, If there is sequence complementarity, nucleic acid molecules will anneal with each other. In one example, a thermostable polymerase may be used to fill in gaps and a thermostable ligase may be provided to seal nicks. Alternatively, the annealed nucleic acid product may be directly used to transform a host cell and gaps and nicks will be repaired “in vivo” by endogenous enzymatic activities of the transformed cell. Single-stranded binding proteins, such as T4 gene 32 protein and RecA, as well as other nucleic acid binding or recombination proteins known in the art, may be included, for example, to facilitate the annealing of nucleic acid molecules.

Where nucleic acid molecules are assembled or cloned into a target vector, in many instances high-copy number vectors may be used for cloning to obtain high yields of a desired polynucleotide. Common high-copy number vectors include pUC (~500-700 copies), PBLUESCRIPT® or PGEM® (~300-500 copies, respectively) or derivatives thereof. In some instances, low-copy number vectors may be used, for example where high expression of a given insert may be toxic for the transformed cell. Such low-copy number vectors with copy numbers of between about 5 and about 30 include for example pBR322, various pET vectors, pGEX, pColE1, pR6K, pACYC or pSC101.

An exemplary list of vectors that can be used in any of the assembly or cloning methods disclosed herein, includes the following: BACULODIRECT™ Linear DIMA; BACULODIRECT™ Linear; DNA Cloning Fragment DNA; BACULODIRECT™ N-term Linear DNA_verA; BACULODIRECT™ C-Term Baculovirus Linear DNA; BACULODIRECT™ N-Term Baculovirus Linear DNA; CHAMPION™ pET100/D-TOPOR; CHAMPION™ pET 101/D-TOPO®; CHAMPION™ pET 102/D-TOPOR; CHAMPION™ pET 104/D-TOPOR; CHAMPION™ pET104-DEST; CHAMPION™ pET151/D-TOPOR; CHAMPION™ pET 160/D-TOPOR; CHAMPION™ pET 160-DEST; CHAMPION™ pET 161-DEST; CHAMPION™ pET200/D-TOPOR; pAc5.1/V5-His A, B, and C; pAd/CMVA/5 DEST; pAd/PL-DEST; pAO815; pBAD/glll A, B, and C; pBAD/His A, B, and C; pBAD/myc-His A, B, and C; pBAD/Thio-TOPOR; pBAD 102/D-TOPOR; pBAD20/D-TOPOR; pBAD202/D-TOPOR; pBAD DEST49; PBAD-TOPO; PBAD-TOPOR; pBlueBac4.5; pBlueBac4.5A/5-His TOPOR; pBlueBacHis2 A, B, and C; pBR322; pBudCE4.1; pcDN3.1A/5-His-TOPO; pcDNA3.1(−); pcDNA3.1(+); pcDNA3.1(+)/myc-HisA; pcDNA3.1(+)/myc-His A, B, C; pcDNA3.1(+)/myc-His B; pcDNA3.1(+)/myc-HisC; pcDNA3.1/His A; pcDNA3.1/His B; pcDNA3.1/His C; pcDNA3.1/Hygro (−); pcDNA3.1/Hygro (+); pcDNA3.1/NT-GFP-TOPO; pcDNA3.1/nV5-DEST; pcDNA3.1A/5-His A; pcDNA3.1A/5-His B; pcDNA3.1A/5-His C; pcDNA3.1/Zeo(−); pcDNA3.1/Zeo(+); pcDNA3.1/Zeo(+); pcDNA3.1DA/5-His-TOPO; pcDNA3.2/V5-DEST; pcDNA3.2A/5-GW/D-TOPO; pcDNA3.2-DEST; pcDNA4/His A; pcDNA4/His B; pcDNA4/His C; pcDNA4/HisMax-TOPO; pcDNA4/HisMax-TOPO; pcDNA4/myc-His A, B, and C; pcDNA4/TO; pcDNA4/TO; pcDNA4/TO/myc-His A; pcDNA4/TO/myc-His B; pcDNA4/TO/myc-His C; pcDNA4/V5-His A, B, and C; pcDNA5/FRT; pcDNA5/FRT; pcDNA5/FRT/TO/CAT; pcDNA5/FRT/TO-TOPO; pcDNA5/FRT/V5-His-TOPO; pcDNA5/TO; pcDNA6.2/cGeneBLAzer-DEST_verA_sz; pcDNA6 2/cGeneBLAzer-GW/D-TOPO pcDNA6; 2/cGeneBlazer-GW/D-TOPO_verA_sz pcDNA6.2/cLumio-DEST; pcDNA6 2/cLumio-DE sTverAsz; pcDNA6.2/GFP-DEST_verA_sz; pcDNA-DEST40; pcDNA-DEST47; pcDNA-DEST53; pCEP4; pCEP4/CAT; pCMV/myc/cyto; pCMV/myc/ER; pCMV/myc/mito; pCMV/myc/nuc; pCMVSPORT6 Notl-Sall Cut; pCoBlasi; pCR Blunt; pCR XL TOPO; pCR®T7/CT TOPOR; pCR®T7/NT TOPO®; pCR2.1-TOPO; pCR3.1; pCR3.1-Uni; pCR4BLUNT-TOPO; pCR4-TOPO; pCR8/GW/TOPO TA; pCR8/GW-TOPO_verA_sz; pCR-Blunt II-TOPO;-pCRII-TOPO; pDEST™ R4-R3; pDEST™10; pDEST™14; pDEST™15; pDEST™17; pDEST™20; pDEST™22; pDEST™24; pDEST™26; pDEST™27; pDEST™32; pDEST™8; pDEST™38; pDEST™39; pDisplay; PDONR™ P2R P3; PDONR™ P2R-P3; PDONR™ P4-P1R; PDONR™ P4-PIR; PDONR™/Zeo; PDONR™/Zeo; PDONR™201; PDONR™207; PDONR™221; PDONR™222; pEF/myc/cyto; pEF/myc/mito; pEF/myc/nuc; pEFi/His A, B, and C; pEF1/myc-His A, B, and C; pEF1/V5-HisA, B, and C; pEF4/myc-His A, B, and C; pEF4/V5-His A, B, and C; pEF5/FRT V5 D-TOPO; pEF5/FRT/V5-DEST™; pEF6/His A, B, and C; pEF6/myc-His A, B, and C; pEF6/V5-His A, B, and C; pEF6A/5-His-TOPO; pEF-DEST51; PENTR™ U6_verA_sz; PENTR™/HirTO_verA_sz; PENTR™-TEV/D-TOPO; PENTR™/D-TOPO; PENTR™/D-TOPO; pHybLex/Zeo; pHyBLex/Zeo-MS2; pIB/His A, B, and C; pIBA/5-His Topo; pIBA/5-His-DEST; plBA/5-His-TOPO; plZA/5-His; plZT/V5-His; plenti4 BLOCK-iT-DEST; pLenti4/BLOCK-iT-DEST; pLenti4/TOA/5-DEST; pThioHis A, B, and C; pTracer-CMV/Bsd; pTracer-CMV2; pTracer-EF A, B, and C; pTracer-EF/Bsd A, B, and C; pTracer-SV40; pTrcHis A, B. and C; pTrcHis2 A, B, and C; pTrcHis2-TOPOR; pTrcHis2-TOPO®; pTrcHis-TOPOR; pT-Rex-DEST30; pT-Rex-DEST30; pT-Rex-DEST™ 31; pT-rEx™-DEST31; pUB/BSD TOPO; pUB6A/5-His A, B, and C; pUC18; pUC19; pUni/V5 His TOPO; pVAX1; pVP22/myc-His TOPO®; pVP22/myc-His2 TOPOR; pYC2.1-E; pYC2/CT; pYC2/Nt A, B. C; pYC2-E; pYC6/CT; pYD1; pYES2; pYES2.1A/5-His-TOPO; pYES2/CT; pYES2/NT; pYES2/NT A, B, & C; pYES3/CT; pYES6/CT; pYES-DEST™ 52; pYESTrp; pYESTrp2; pYESTrp3; pZeoSV2; pZeoSV2(+); PZERO™-1; and PZERO™-2.

Where desirable, assembled nucleic acid molecules may be purified or directly inserted into vectors and host cells. PCR-based insertion into a target vector may be appropriate when the desired construct is fairly small (e.g., less than 5 kilobases). Thus, in some examples, a target vector may have a limited size to allow for PCR-mediated elongation of the full-length fusion construct as disclosed e.g., in PCT publication No. WO 2020/001783. Under certain conditions, full-length elongation and/or amplification of the fusion construct may not be required. In such circumstances, the size of the target vector may not be limiting. More generally suitable target vectors may have a size of between about 0.5 and about 20 kb, or between about 1 kb and about 10 kb, or between about 2 kb and about 5 kb.

In some examples, assembly products may be cloned into or assembled with a linearized vector. A vector may be linearized by any means including PCR amplification of a closed circular template vector molecule. Alternatively, the vector may be linearized by restriction enzyme cleavage with one or more enzymes producing either blunt or sticky ends. Such enzymes include restriction endonucleases of type II which cleave nucleic acid at fixed positions with respect to their recognition sequence. Restriction enzymes that can be selected to produce either “blunt” or “sticky” ends upon cleavage of a double-stranded nucleic acid are known to those skilled in the art and can be selected by the skilled person depending on the vector sequence and assembly requirements. In some instances, a vector may be linearized using a type II restriction endonuclease that generates blunt ends. Such endonucleases include, e.g., ScaI, SmaI, HpaI, HincII, HaeII and AluI. Other means to cleave or linearize a vector include the use of site-specific engineered nucleases that induce double-strand breaks in a given nucleic acid molecule, such as Transcription activator-like effector nuclease (often referred to as TALEN), CRISPR/Cas9 nuclease, Zinc-finger nuclease or Argonaute protein-nuclease.

Assembled nucleic acid molecules may also include functional elements which confer desirable properties. These elements may either be provided by the plurality of retrieved amplification products or by the target vector. Examples of such elements include origins of replication, long terminal repeats, resistance markers (such as antibiotic resistance genes), selectable markers and antidote coding sequences (e.g., ccdA coding sequences for counter-acting toxic effects of ccdB), promoters, enhancers, polyadenylation signal coding sequences, 5′ and 3′ UTRs and other components suitable for the particular use(s) of the nucleic acid molecules (e.g., enhancing mRNA or protein production efficiency). In examples where nucleic acid molecules are assembled to form an operon, the assembled nucleic acid products will often contain promoter and terminator sequences. Furthermore, assembled nucleic acid molecules may contain multiple cloning sites, such as, e.g., type II or type IIs cleavage sites and/or GATEWAY® recombination sites, as well as other sites for the connection of nucleic acid molecules to each other or subsequent higher order or hierarchical assembly.

In some aspects of the disclosure, assembled nucleic acid molecule length will vary from about 20 base pairs to about 10,000 base pairs, from about 100 base pairs to about 5,000 base pairs, from about 150 base pairs to about 5,000 base pairs, from about 200 base pairs to about 5,000 base pairs, from about 250 base pairs to about 5,000 base pairs, from about 300 base pairs to about 5,000 base pairs, from about 350 base pairs to about 5,000 base pairs, from about 400 base pairs to about 5,000 base pairs, from about 500 base pairs to about 5,000 base pairs, from about 700 base pairs to about 5,000 base pairs, from about 800 base pairs to about 5,000 base pairs, from about 1,000 base pairs to about 5,000 base pairs, from about 100 base pairs to about 4,000 base pairs, from about 150 base pairs to about 4,000 base pairs, from about 200 base pairs to about 4,000 base pairs, from about 300 base pairs to about 4,000 base pairs, from about 500 base pairs to about 4,000 base pairs, from about 50 base pairs to about 3,000 base pairs, from about 100 base pairs to about 3,000 base pairs, from about 200 base pairs to about 3,000 base pairs, from about 250 base pairs to about 3,000 base pairs, from about 300 base pairs to about 3,000 base pairs, from about 400 base pairs to about 3,000 base pairs, from about 600 base pairs to about 3,000 base pairs, from about 800 base pairs to about 3,000 base pairs, from about 100 base pairs to about 2,000 base pairs, from about 200 base pairs to about 2,000 base pairs, from about 300 base pairs to about 1,500 base pairs, etc.

In some aspects, the length of nucleic acid molecules generated by methods of the disclosure having a desired sequence (including amplified and/or assembled nucleic acid fragments) is at least 100, 200, 300, 400, 500, or 750 bases or 1 kb, 2.5 kb, 5 kb, 7 kb, 10 kb, 100 kb, 250 kb, 500 kb, 750 kb, 1 Mb, 2.5 Mb, 5 Mb, 10 Mb, 20 Mb, 30 Mb, 40 Mb, 50 Mb, 75 Mb, 100 Mb, 200 Mb, 300 Mb or 500 Mb. In some aspects, the length of the polynucleotide is from about 0.1 kb to about 500 Mb, 250 Mb, 100 Mb, 75 Mb, 50 Mb, 25 Mb, 20 Mb, 15 Mb, 10 Mb, 5 Mb, 500 kb, 250 kb, 100 kb, 75 kb, 50 kb, 25 kb, 20 kb, 15 kb, 10 kb, 5 kb, or from about 0.2 kb to about 100 kb, from about 0.1 kb to about 30 kb, or from about 0.5 kb to about 30 kb, 20 kb, 10 kb, 5 kb, or 4 kb. In some embodiments, the size ranges for nucleic acid molecules produced according to methods disclosed herein are any of the ranges provided herein. A skilled person will recognize that methods provided herein are flexible and can produce desired nucleic acid molecules of a wide range of lengths depending on the number and length of the nucleic acid molecules in the starting mixtures.

In many instances, nucleic acid molecules prepared by methods of the disclosure will be replicable. Further, many of these replicable nucleic acid molecules will be circular (e.g., plasmids). Replicable nucleic acid molecules, regardless of whether they are circular, will generally be formed from the assembly of two or more (e.g., three, four, five, eight, ten, twelve, etc.) nucleic acid fragments. In some instances, methods of the disclosure employ selection based upon the reconstitution of one or more (e.g., two, three, four, etc.) selection marker or one or more (e.g., two, three, four, etc.) origin of replication resulting from the linking of different nucleic acid fragments. Further selection may result from the formation of a circular nucleic acid molecule, in instances where circularity is required for replication.

Functional elements may be used to select for correctly assembled fusion constructs. For example, the target vector may provide one or more truncated and therefore inactive versions of certain elements (such as, e.g., a portion of an origin of replication or a portion of a selection marker) which need to be completed by flanking sequences provided by complementary oligonucleotides during the assembly procedure. Such positive selection approach which renders only clones comprising correctly assembled functional elements is described in Baek et al., Analytical Biochemistry 476, p. 1-4 (2015).

Error-free target nucleic acid molecules identified based on the in vitro cloning procedures disclosed herein may be further combined with other assembly products or nucleic acid molecules obtained from other sources to assemble even larger nucleic acid molecules (e.g., genes, operons, pathways etc.). For example, one or more error-free nucleic acid molecules obtained from a first UMI-cloning method may be combined/assembled with one or more error-free nucleic acid molecules obtained from a second UMI-cloning method using anyone of the various assembly technologies disclosed herein.

Larger nucleic acid molecules may also be assembled in vivo. In in vivo assembly methods, a mixture of all of the fragments to be assembled is often used to transfect the host cell using standard transfection techniques. The ratio of the number of molecules of fragments in the mixture to the number of cells in the culture to be transfected should be high enough to permit at least some of the cells to take up more molecules of sub-fragments than there are different sub-fragments in the mixture. Thus, in most instances, the higher the efficiency of transfection, the larger number of cells will be present which contain all of the nucleic acid fragments required to form the final desired assembly product. Technical parameters along these lines are set out in U.S. Patent Publication No. 2009/0275086 A1.

Large nucleic acid molecules will typically be 20 kb or larger (e.g., larger than 25 kb, larger than 35 kb, larger than 50 kb, larger than 70 kb, larger than 85 kb, larger than 100 kb, larger than 200 kb, larger than 500 kb, larger than 700 kb, larger than 900 kb, etc.). Methods for producing and even analyzing large nucleic acid molecules are known in the art. For example, Karas et al., Journal of Biological Engineering 7:30 (2013) shows the assembly of an algal chromosome in yeast and pulse-field gel analysis of such large nucleic acid molecules.

One group of organisms known to perform homologous recombination fairly efficient is yeasts (e.g., Saccharomyces cerevisiae, Schizosaccharomyces pombe, Pichia, pastoris, etc.). Yeast hosts are particularly suitable for manipulation of donor genomic material because of their unique set of genetic manipulation tools. The natural capacities of yeast cells, and decades of research have created a rich set of tools for manipulating DNA in yeast. These advantages are well known in the art. For example, yeast, with their rich genetic systems, can assemble and re-assemble nucleotide sequences by homologous recombination, a capability not shared by many readily available organisms. Yeast cells can be used to clone larger pieces of DNA, for example, entire cellular, organelle, and viral genomes that are not able to be cloned in other organisms. Thus, in some examples, the disclosure employs the enormous capacity of yeast genetics generate large nucleic acid molecules (e.g., synthetic genomics) by using yeast as host cells for assembly and maintenance.

Exemplary of the yeast host cells are yeast strain VL6-48N, developed for high transformation efficiency parent strain: VL6-48 (ATCC Number MYA-3666™)), the W303a strain, the MaV203 strain (Thermo Fisher Scientific, cat. no. 11281-011), and recombination-deficient yeast strains, such as the RAD54 gene-deficient strain, VL6-48-Δ 54G (MAT α his3-Δ 200 trp 1-Δ 1 ura3-52 lys2 ade2-101 met14 rad54-Δ 1::kanMX), which can decrease the occurrence of a variety of recombination events in yeast artificial chromosomes (YACs).

In some instances, error-free target nucleic acid molecules identified based on the in vitro cloning procedures disclosed herein may be further assembled into synthetic circular or linear nucleic acid molecules. Assembled synthetic circular or linear nucleic acid molecules may further be amplified wherein such amplifying may comprise rolling circle amplification, strand displacement amplification, or polymerase chain reaction.

An exemplary process for generating synthetic circular DNA vectors may comprise the following steps: a) amplification of a supercoiled monomeric nucleic acid template using phi29 polymerase to generate linear concatemeric DNA having a restriction site that defines the boundaries between repeated DNA fragments. The concatamers can then be digested using a restriction enzyme that cleaves the DNA into unit-length fragments. Next, DNA ligase is added to induce self-ligation of the DNA fragments, generating a mixture of DNA structures including open relaxed circles and supercoiled DNA monomers. This mixture can be column-purified using a thiophilic aromatic adsorption chromatography resin (e.g, Plasmidselect Xtra, GE Healthcare 28-4024-01), which has a selectivity that allows supercoiled covalently closed circular forms of plasmid DNA to be separated from open circular forms. Supercoiled DNA monomer obtained from this purification can be recovered and used in therapeutic workflows or, alternatively, may serve as a template for additional amplification.

In some examples, the assembly products may be linear covalently closed structures also referred to as “doggybone DNA” or dbDNA™ according to methods described e.g., in U.S. Pat. No. 9,109,250. In this cell-free procedure a circular DNA template (e.g., containing one or more of the nucleic acid molecules generated as described herein) that comprises at least one protelomerase target sequence is denatured and replicated by rolling circle amplification in the presence of a phi 29 DNA polymerase and one or more primers. The amplified DNA product is then contacted with a protelomerase under conditions promoting production of closed linear DNA. In examples of closed linear DNA molecules hairpin loops flank complementary base-paired DNA strands, forming a “doggy-bone” shaped structure.-target backbone sequences may then be removed and degraded using restriction enzymes and exonucleases. dbDNA is particularly suitable for therapeutic uses because it does not comprise problematic bacterial sequences such as antibiotic resistance genes.

In some examples, the synthetic circular or linear nucleic acid molecule may be a DNA vector.

### APPLICATIONS AND USES

Generally, methods of the invention allow for the de novo production of completely “synthetic” nucleic acid molecules thereby circumventing traditional cloning steps that require cells. Thus, the nucleic acids generated by methods of the disclosure may be used in workflows that are subject to high quality standards regarding sequence correctness and absence of any cell-derived products (such as e.g., endotoxin-free). In addition, in vitro production of nucleic acid molecules according to methods of the invention allows for a high degree of automation and standardization making the methods particularly suitable for therapeutic nucleic acids production platforms.

In many instances, a nucleic acid molecule obtained by any method disclosed herein may be used as template in an in vitro transcription reaction, in nucleic acid sequencing molecular cloning, medical diagnostics, a nucleic acid vaccine, a self-replicating system, the assembly of a DNA nanostructure, a diagnostic assay, or a biosensor and the like.

In some embodiments, the nucleic acids obtained by the disclosed methods may be used as templates for production of mRNA molecules or polypeptides. In some embodiments, the mRNA molecules are produced using cell-free methods (e.g., in vitro transcription or cell-free RNA synthesis as described e.g., in US Patent Publications No. 2017/0292138 or 2018/0087045 or 2019/0144489). In some embodiments, a nucleic acid obtained by methods of the invention used as template for RNA production may comprise a 5′ UTR, a nucleic acid sequence encoding one or more antigens or therapeutic targets, and a 3′ UTR, in addition to RNA production elements (e.g., a T7 promoter, digestion sites, etc.), and optionally, one or more restriction digestion sites for template linearization.

In some embodiments a nucleic acid template comprising sequence-verified nucleic acid molecules obtained from methods of the invention may encode one or more therapeutic molecules or portions thereof (i.e., nucleic acids, peptides, proteins, neoantigens etc.). Such therapeutic molecules may be used to, for example, replace a gene, mRNA or protein that is deficient or abnormal, augment an existing biological pathway, provide a novel function or activity, in a cell, tissue, organ, or subject. Therapeutic molecules may also be used to elicit an immune response. Exemplary types of therapeutic templates include, but are not limited to, sequences encoding enzymes, co factors, carrier proteins, transport proteins, cytokines, signaling proteins, suicide gene products, drug resistance proteins, tumor suppressor protein hormones, peptides with immunomodulatory properties, tolerogenic peptides, i peptides, antigens, neoantigens, antibodies and antigen-binding fragments thereof, anti-oxidant molecules, engineered immunoglobulin-like molecules, fusion proteins, immune co-stimulatory molecules, immunomodulatory molecules, chimeric antigen receptors, toxins, tumor suppressor proteins, growth factors, membrane proteins, receptors, vasoactive proteins, ligand proteins, antiviral proteins, ribozymes, RNAs, riboswitches, mRNA, RNA interference (e.g., shRNA, siRNA, microRNA) molecules, and derivatives thereof.

Gene therapy involves transduction of heterologous genes into target cells to correct a genetic defect underlying a disorder in a subject. A variety of transduction approaches have been developed for use in gene therapy over the past several decades. For example, traditional bacterial plasmid DNA vectors represent a versatile tool in gene delivery but can present limitations owing to their bacterial origin. Plasmid DNA vectors include bacterial genes, such as antibiotic resistance genes and origins of replication. Additionally, plasmid DNA vectors include bacterial signatures, such as CpG motifs. In addition, the use of bacterial expression systems for producing plasmid DNA vectors involves the risk of introducing contaminating impurities from the bacterial host, such as endotoxins or bacterial genomic DNA and RNA, which can lead to loss of gene expression in vivo, e.g., by transcriptional silencing. Thus, in some embodiments a DNA vector comprising one or more nucleic acid molecules obtained by methods disclosed herein lacks one or more of these bacterial elements such as an origin of replication, a drug or antibiotic resistance gene, CpG motifs and/or a site-specific recombination recognition site.

### EXAMPLES

### Example 1: Monte Carlo Simulation of In Vitro Cloning Concept

A Monte Carlo simulation was conducted to determine the optimal number of starting nucleic acid molecules resulting in a sufficient amount of compartments with single families (i.e., having only one specific amplification product) while restricting the amount of compartments with two or more families. The simulation was based on the following assumptions: nucleic acid fragment lengths of 1,000 bp and an estimated error rate of 1 in 3,000 bp. Under these assumptions it is expected that about 3 clones would need to be “picked” to find a correct one in 98% of cases. Further, the simulation used the following parameters: a set of 192 5′ tags and 192 3′ tags each having a different barcode, thus yielding a total of 384 different barcodes which results in approximately 37,000 different 5′/3′ barcode combinations. Furthermore, it was assumed that 24 wells of a 96-well plate are pre-loaded with respective barcode primer pairs. FIG. 7A shows a simulation with exactly 36,864 molecules per well which yields a comparable number of 8 “empty” wells having no amplification product (where the pre-loaded primer pair does not match with any of the barcode combinations present in the well) and 8 wells having only one specific amplification product (where the pre-loaded primer pair matches with exactly one tagged nucleic acid molecule present in a well), whereas 6 wells would have two or more amplification products (where the pre-loaded primer pair matches with two or more tagged nucleic acid molecules present in a well). Thus, after removing the “empty” wells, approximately 60% of picked samples would be single clones (i.e., 8/14). FIG. 7B shows a simulation with a dilution to 20% less molecules resulting in too many empty wells, whereas FIG. 7C shows a simulation with too many molecules (+20%) resulting in too many wells with more than one specific amplification product. This simulation demonstrates that with the correct dilution (to achieve a predetermined number of nucleic acid molecules per well) almost all experiments will yield 3 or more single clones (with only outliers having less than 3).

### Example 2: In Vitro Cloning with Sanger Sequencing

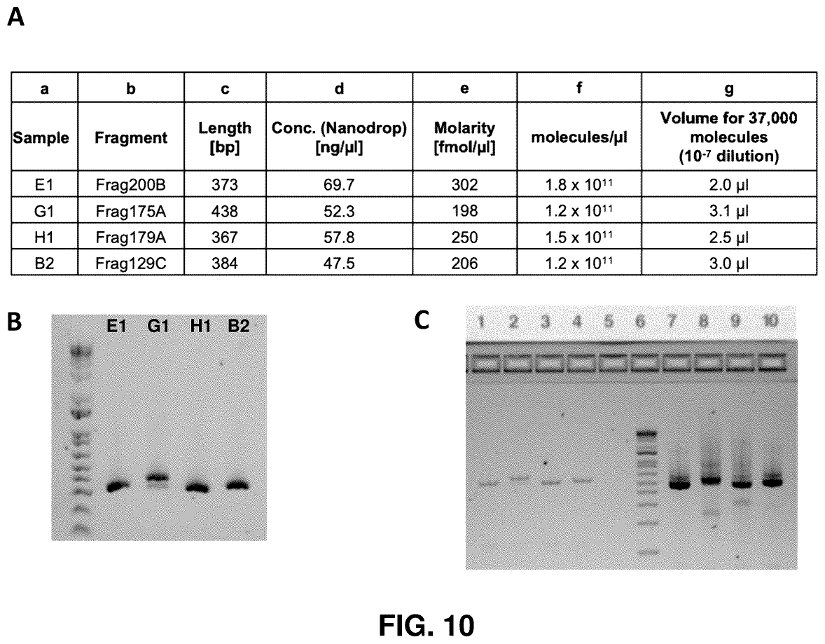

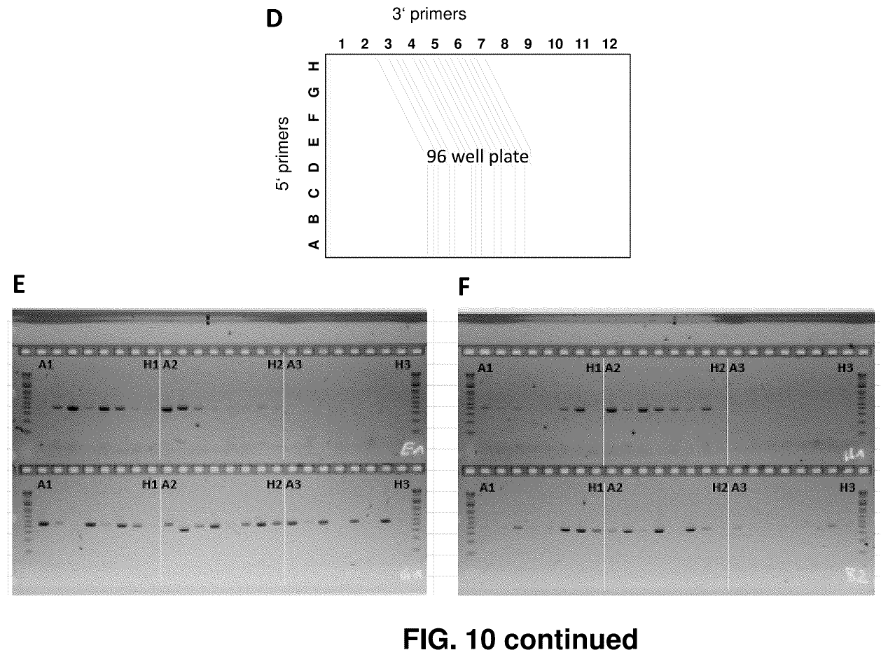

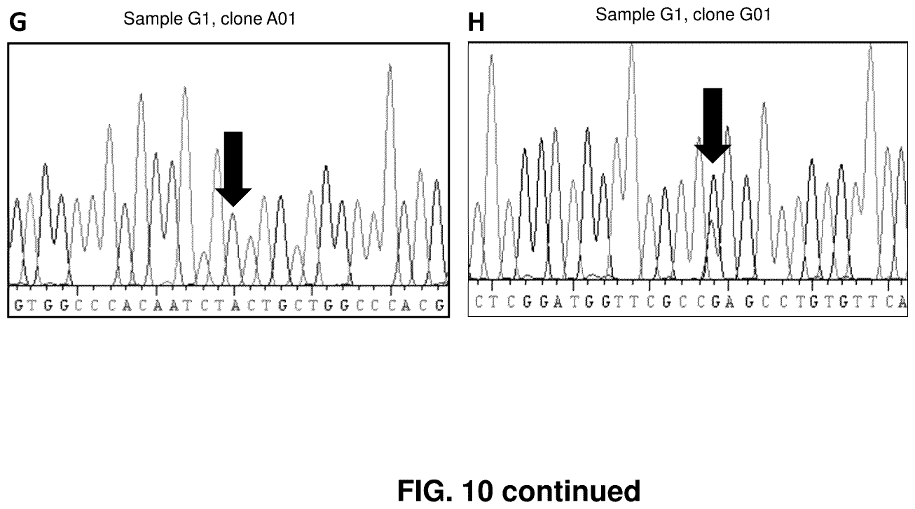

An overview of the workflow used in this Example is shown in FIG. 9. Four nucleic acid fragments having sizes of 373 bp, (Frag200B), 438 bp (Frag175A), 367 bp (Frag179A) and 384 bp (Frag129C) referred to as samples E1, G1, H1 and B2, respectively (see FIG. 10A, col. a-c), were assembled from 50 to 60-mer oligonucleotides by overlap extension PCR and purified with Ampure XP beads (Beckman Coulter). All fragments were designed with universal 18-bp linkers at both termini. 1 μl of purified fragment DNA was analysed on an E-Gel™ 1% agarose gel (Thermo Fisher Scientific Inc.) to validate fragment sizes (FIG. 10B; lane 1:5 μl E-Gel™ 1 kb Plus DNA Ladder; Thermo Fisher Scientific Inc.). The concentration of all four nucleic acid samples was then measured using a NanoDrop™ 8000 instrument (Thermo Fisher Scientific) and molarities of samples were determined (FIG. 10A, col. d-e), yielding approx. 1.2-1.8×10¹¹ molecules per μl sample (FIG. 10A, col. f). Thus, a 1:10¹¹ dilution of the samples was expected to contain on average 1 nucleic acid molecule per μl. To verify the concentration measurement, 1:10⁸ to 1:10¹² dilution series of all four samples were obtained and validated by qPCR. A random distribution of amplification versus no amplification was achieved at a dilution of 1:10¹¹ indicating accuracy of concentration measurements by NanoDrop™ 8000.

In vitro cloning was then conducted with fragment dilutions in PCR cups adjusted to obtain approx. 37,000 nucleic acid molecules per cup (FIG. 10A, col. g), which was expected to provide a nearly equal distribution of empty wells (no amplification product), clonal wells (single amplification product) and non-clonal wells (multiple amplification products) upon amplification of tagged molecules with barcode primers. Accordingly, 192 5′ barcode tags and 192 3′ barcode tags (yielding 192×192=36,864 calculated barcode combinations) were designed with 5′ amplification handles and 3′ linker-complementary regions and were provided HPLC-purified (ELLA Biotech). A 2-cycle PCR reaction with the 192×192 barcode tags was conducted to tag the approx. 37′000 nucleic acid molecules using Platinum SuperFi™ DNA polymerase (Thermo Fisher Scientific). Tagged samples were then amplified into families in a 25-cycle PCR reaction with amplification primers designed to hybridize to the amplification handles at both ends of the tagged nucleic acid molecules. 5 μl of the tagged and amplified samples were analysed on a 2% E-Gel™ (Thermo Fisher Scientific) and compared to positive controls using 1:100 sample dilutions with approximately 3.7×10⁹ template nucleic acid molecules in the tagging reaction. (FIG. 10C, lanes 1-4: tagged and amplified samples of 37,000 molecule dilutions of E1, G1, H1 and B1; lane 5: no template control; lane 6:5 μl of E-Gel™ 1 Kb Plus DNA Ladder; lanes 7-10: tagged and amplified samples of 3,7×10⁹ molecule dilutions of E1, G1, H1 and B1, respectively). An aliquot of each amplification product was then transferred into wells of a 96-well plate pre-loaded with PCR mastermix and defined 5′ and 3′ barcode primers (FIG. 10D) and amplified using SuperFi™ polymerase (Thermo Fisher Scientific). 5 μl of each PCR reaction was then analysed on a 2% E-Gel (FIGS. 10E and F, columns 1-3 from each PCR plate) indicating that those wells that contained a “matching” barcode primer pair yielded amplification products, whereas some wells were tested negative. In sum, amplification products were obtained in 58% of the wells for fragment E1, 79% of the wells for fragment G1, 54% of the wells for fragment H1 and 79% of the wells for fragment B2, respectively. Samples were then Sanger-sequenced using primers binding to the universal 18-bp linkers at both termini of the original fragments revealing samples that were likely clonal and samples that were clearly not clonal. FIG. 10G shows exemplary well A01 of sample G1 comprising a likely clonal sequence: The arrow indicates a sequence error compared to the expected sequence of the synthesized fragment. The sequence is likely clonal because a second sequence type present in the well would have a high probability of showing a wild-type peak at the position of the error. (Sanger sequencing technology does not allow detection of contaminations at concentrations lower than approximately 5% of the total DNA amount, however). FIG. 10H shows exemplary well G01 of sample G1 comprising a mixed sequence as indicated by the double peak in the chromatogram indicated by the arrow.

In order to determine clonality of the reaction products, the PCR products from 8 wells per original fragment were cloned into pCRTMBlunt II-TOPO™ vector (Thermo Fisher Scientific) and 8 TOPO-clones were sequenced for each of the 32 wells. As shown in Table 1, clonal wells were identified for all original fragments. The high number of non-clonal wells and the low number of wells yielding no valid insert indicate that the actual number of template molecules used in the tagging reaction was slightly too high due to inaccurate dilution, which confirms that obtaining the predetermined number of nucleic acid molecules as illustrated in FIG. 7 is critical. To obtain the predetermined number of molecules, the final dilution could be measured by digital PCR.

TABLE 1

| Sample/Fragment | Well | clonal? | comment |
|---|---|---|---|
| E1/Frag200B | A2 | no | 5 different variants in 8 clones |
| | B2 | likely | all correct |
| | C2 | no | 2 different variants in 8 clones |
| | D2 | yes | all with same substitution |
| | E2 | no | 4 different variants in 8 clones |
| | F2 | no | 3 different variants in 8 clones |
| | G2 | poor sequence quality | |
| | H2 | no colonies | |
| G1/Frag175A | A2 | no | 3 different variants in 8 clones |
| | B2 | no | 2 different variants in 7 clones |
| | C2 | no | 3 different variants in 8 clones |
| | D2 | yes | all with same substitution |
| | E2 | no | 4 different variants in 8 clones |
| | F2 | likely | 7 clones correct, 1 too short in cPCR |
| | G2 | no | 2 different variants in 7 clones |
| | H2 | no | 6 different variants in 8 clones |
| H1/Frag179A | A2 | no | 3 different variants in 8 clones |
| | B2 | no | 2 different variants in 8 clones |
| | C2 | no | 2 different variants in 8 clones |
| | D2 | no | 2 different variants in 8 clones |
| | E2 | likely | all correct |
| | F2 | no | 3 different variants in 8 clones |
| | G2 | no | 2 different variants in 6 clones |
| | H2 | yes | all with same 2 substitutions |
| B2/Frag129C | A2 | no | 5 different variants in 8 clones |
| | B2 | no | 2 different variants in 8 clones |
| | C2 | no | 2 different variants in 8 clones |
| | D2 | no | 2 different variants in 8 clones |
| | E2 | likely | all correct |
| | F2 | No | 3 different variants in 3 clone |
| | G2 | No | 2 different variants in 6 clones |
| | H2 | likely | all correct |

Further aspects of the disclosure are exemplified by the following numbered clauses:
Clause 1. A method for identifying a nucleic acid molecule having a desired sequence in a mixture of nucleic acid molecules comprising the following steps: (a) providing one or more mixtures of nucleic acid molecules, each mixture comprising a plurality of nucleic acid molecules designed to have a desired sequence, wherein each nucleic acid molecule optionally comprises a linker at the 5′ end and at the 3′ end; (b) providing a set of nucleic acid tags, each tag comprising at least (i) a barcode, and (ii) a handle or adapter at the 5′ end; (c) optionally determining the concentration of nucleic acid molecules in each of the one or more mixtures; (d) providing one or more first compartments and diluting each of the one or more mixtures of nucleic acid molecules in a separate first compartment to obtain diluted mixtures of nucleic acid molecules, each diluted mixture comprising a predetermined amount of nucleic acid molecules; (e) contacting the diluted mixtures of nucleic acid molecules in one or more of the first compartments with the set of nucleic acid tags and attaching a tag to both ends of substantially each nucleic acid molecule in the one or more first compartments to obtain tagged nucleic acid molecules having a barcode region and a handle or adapter region at both ends; (f) optionally providing one or more pairs of amplification primers designed to hybridize to both ends of the tagged nucleic acid molecules and amplifying the tagged nucleic acid molecules in the one or more first compartments; (g) optionally purifying the amplified nucleic acid molecules; (h) providing a set of barcode primers, each barcode primer of the set comprising a barcode-specific region designed to hybridize to a specific barcode in the set of nucleic acid tags; (i) providing one or more second compartments and contacting at least a portion of each of the optionally amplified and optionally purified tagged nucleic acid molecules with a defined pair of the set of barcode primers in a second compartment and performing an amplification reaction in each second compartment to obtain one or more second compartments comprising: (i) one specific amplification product, (ii) no specific amplification product, and/or (iii) two or more specific amplification products; (j) optionally identifying second compartments (ii) having no specific amplification product and/or second compartments (iii) having two or more specific amplification products and excluding such second compartments from subsequent steps; (k) optionally pooling at least a portion of the amplification products from two or more of the second compartments; (l) sequencing the amplification products to obtain sequence data; (m) analysing the sequence data, and (n) identifying one or more second compartments comprising one specific amplification product, thereby identifying one or more nucleic acid molecules having the desired sequence.
Clause 2. A method for identifying a nucleic acid molecule having a desired sequence in a mixture of nucleic acid molecules comprising the following steps: (a) providing one or more mixtures of nucleic acid molecules, each mixture comprising a plurality of nucleic acid molecules designed to have a desired sequence, wherein each nucleic acid molecule optionally comprises a linker at the 5′ end and at the 3′ end; (b) providing a set of nucleic acid tags, each tag comprising at least (i) a barcode, and (ii) a handle or adapter at the 5′ end; (c) contacting the one or more mixtures of nucleic acid molecules with the set of nucleic acid tags and attaching a tag to both ends of substantially each nucleic acid molecule to obtain tagged nucleic acid molecules having a barcode region and a handle or adapter region at both ends; (d) optionally determining the concentration of tagged nucleic acid molecules in one or more mixtures; (e) providing one or more first compartments and diluting each of the one or more mixtures of tagged nucleic acid molecules in a separate first compartment to obtain diluted mixtures of tagged nucleic acid molecules, each diluted mixture comprising a predetermined amount of tagged nucleic acid molecules; (f) optionally providing one or more pairs of amplification primers designed to hybridize to both ends of the tagged nucleic acid molecules and amplifying the tagged nucleic acid molecules in the one or more first compartments; (g) optionally purifying the amplified nucleic acid molecules; (h) providing a set of barcode primers, each barcode primer of the set comprising a barcode-specific region designed to hybridize to a specific barcode in the set of nucleic acid tags; (i) providing one or more second compartments and contacting at least a portion of each of the optionally amplified and optionally purified tagged nucleic acid molecules with a defined pair of the set of barcode primers in a second compartment and performing an amplification reaction in each second compartment to obtain one or more second compartments comprising: (i) one specific amplification product, (ii) no specific amplification product, and/or (iii) two or more specific amplification products; (j) optionally identifying second compartments (ii) having no specific amplification product and/or second compartments (iii) having two or more specific amplification products and excluding such second compartments from subsequent steps; (k) optionally pooling at least a portion of the amplification products from two or more of the second compartments; (l) sequencing the amplification products to obtain sequence data; (m) analysing the sequence data, and (n) identifying one or more second compartments comprising one specific amplification product, thereby identifying one or more nucleic acid molecules having the desired sequence.
Clause 3. The method of any previous clause, wherein the nucleic acid molecules are double-stranded or at least partially double-stranded.
Clause 4. The method of clause 1 or 2, wherein the nucleic acid molecules are linear double-stranded nucleic acid molecules.
Clause 5. The method of any previous clause, wherein the nucleic acid molecules comprise a GC content of from about 35% to about 70%.
Clause 6. The method of any previous clause, wherein the nucleic acid molecules in each mixture have an error rate of less than 1 in 400 bp, preferably less than 1 in 800 bp, more preferably less than 1 in 1,000 bp, less than 1 in 1,500, or less than 1 in 5,000.
Clause 7. The method of any previous clause, wherein the nucleic acid molecules are between about 200 and about 5,000 nucleotides in length.
Clause 8. The method of any previous clause, wherein the nucleic acid molecules are assembled from a plurality of oligonucleotides.
Clause 9. The method of any previous clause, wherein the nucleic acid molecules are obtained by PCR assembly of overlapping single-stranded oligonucleotides.
Clause 10. The method of anyone of clauses 1-8, wherein the nucleic acid molecules are obtained by ligation of double-stranded oligonucleotides.
Clause 11. The method of anyone of clauses 8-10, wherein the oligonucleotides are synthesized on a solid support, optionally wherein the solid support is the surface of a microchip or a microarray and/or wherein the solid support is particles or beads.
Clause 12. The method of anyone of clauses 8-11, wherein the oligonucleotides are between about 20 and about 200 nucleotides in length.
Clause 13. The method of anyone of clauses 8-13, wherein the oligonucleotides are synthesized by chemical, electrochemical, photochemical or enzymatic synthesis.
Clause 14. The method of any previous clause, wherein the linker at the 5′ end and the 3′ end of each nucleic acid molecule is between about 10 and about 50 nucleotides in length.
Clause 15. The method of any previous clause, wherein the linker at the 5′ end and the 3′ end of each nucleic acid molecule further comprises: (i) a universal primer binding site, and/or (ii) a restriction enzyme cleavage site.
Clause 16. The method of clause 15, wherein the universal primer binding site of the linker is between about 10 and about 30 nucleotides in length.
Clause 17. The method of anyone of clauses 15 or 16, wherein the universal primer binding site of the linker at the 5′ end differs in sequence from the universal primer binding site of the linker at the 3′ end.
Clause 18. The method of anyone of clauses 15 to 17, wherein the restriction enzyme cleavage site of the linker is positioned to allow removal of the universal primer binding site from the nucleic acid molecule.
Clause 19. The method of anyone of clauses 15 to 18, wherein the restriction enzyme cleavage site of the linker is a type IIS restriction enzyme cleavage site.
Clause 20. The method of any previous clause, wherein the number of mixtures of nucleic acid molecules is between 2 and about 1,000, between about 10 and about 500 or between about 50 and about 200.
Clause 21. The method of any previous clauses, wherein the tags in the set of nucleic acid tags are single stranded, double-stranded or partially double-stranded.
Clause 22. The method of any previous clauses, wherein the tags in the set of nucleic acid tags are phosphorylated at the 5′ end.
Clause 23. The method of any previous clauses, wherein each tag in the set of nucleic acid tags is between about 30 and about 200 nucleotides in length, or between about 45 and about 90 nucleotides in length.
Clause 24. The method of any previous clauses, wherein each tag in the set of nucleic acid tags further comprises a linker binding region at the 3′ end.
Clause 25. The method of clause 24, wherein the linker binding region is designed to hybridize to the linker at the 5′ end and/or the 3′ end of each nucleic acid molecule.
Clause 26. The method of anyone of clause 24 or 25, wherein the linker binding region is between about 10 and about 50 nucleotides in length.
Clause 27. The method of any previous clause, wherein the set of nucleic acid tags comprises a first subset of tags for attachment to the 5′ end of the nucleic acid molecules and a second subset of tags for attachment to the 3′ end of the nucleic acid molecules.
Clause 28. The method of clause 27, wherein the first and second subset of nucleic acid tags each comprise between about 100 and about 1,000 different barcodes or between about 200 and about 500 different barcodes.
Clause 29. The method of clause 27 or 28, wherein a combination of the first and second subset of tags results in between about 10,000 and about 100,000 different barcode combinations.
Clause 30. The method of any one of clauses 27 to 29, wherein the first and second subset of nucleic acid tags result in between about 10×10 and about 200×200 different barcode combinations, or result in about 192×192 different barcode combinations.
Clause 31. The method of any previous clause, wherein the barcode of each tag is between about 5 and about 30 nucleotides in length.
Clause 32. The method of any previous clause, wherein the plurality of barcodes of the set of nucleic acid tags have a Levenshtein distance of minimum 2 to 5 and/or have a melting temperature of between 50 °C and 55 °C.
Clause 33. The method of any one of clauses 27 to 32, wherein each barcode of at least one subset of nucleic acid tags comprises at least one, at least 2, or between about 3 and about 10 degenerate nucleotide positions, optionally wherein the degenerate nucleotide positions are located at the 3′ end of the barcode.
Clause 34. The method of any previous clause, wherein the handle comprises a universal primer binding site.
Clause 35. The method of any previous clause, wherein the handle is between about 10 and about 30 nucleotides in length.
Clause 36. The method of any previous clause, wherein the adapter is between about 20 and about 50 nucleotides in length.
Clause 37. The method of clause 1 or anyone of clauses 3 to 6, wherein in step (c) the concentration of nucleic acid molecules in each mixture is determined by qPCR, digital PCR, optical measurement, spectrophotometry or fluorometric detection.
Clause 38. The method of any previous clause, wherein determining the concentration of nucleic acid molecules in one or more mixtures comprises qPCR, digital PCR, optical measurement, spectrophotometry or fluorometric detection.
Clause 39. The method of any previous clause, wherein the one or more first compartments are selected from wells of a microwell plate, chambers of a cartridge, vessels, or columns.
Clause 40. The method of any previous clause, wherein the one or more first compartments have a volume of between about 0.1 μl and 1 ml, preferably between about 10 μl and about 200 μl.
Clause 41. The method of any previous clause, wherein the one or more first compartments are 96-well plates or 384-well plates.
Clause 42. The method of any previous clause, wherein the diluting comprises generating serial dilutions of each mixture of nucleic acid molecules and selecting a dilution determined to comprise a desired number of nucleic acid molecules.
Clause 43. The method of any previous clause, wherein the diluting comprises transferring or pipetting a calculated volume of each mixture of nucleic acid molecules into each first compartment, optionally wherein the transferring is conducted using an acoustic liquid handling device.
Clause 44. The method of any previous clause, wherein the diluting comprises diluting each mixture of nucleic acid molecules by about 1:10,000 and about 1:10,000,000,000 or generating serial dilutions of between about 10⁻⁴ and about 10⁻¹⁰ of each mixture.
Clause 45. The method of any previous clause, wherein the predetermined amount of nucleic acid molecules in each first compartment comprises between about 20 and about 1,000,000, preferably between about 20,000 and about 80,000 and more preferably between about 30,000 and about 50,000 molecules.
Clause 46. The method of any previous clause, wherein the predetermined amount of nucleic acid molecules in each first compartment represents the number of barcode combinations of the first set of nucleic acid tags.
Clause 47. The method of clause 1 or anyone of clauses 3 to 46, wherein the contacting comprises adding a portion of the first set of nucleic acid tags to each one of the one or more first compartments comprising a diluted mixture of nucleic acid molecules.
Clause 48. The method of any previous clause, wherein the attaching is performed by PCR, optionally wherein the number of PCR cycles is limited to 2.
Clause 49. The method of anyone of clauses 1 to 46, wherein the attaching is performed by ligation in the presence of a ligase.
Clause 50. The method of any previous clause, wherein the amplifying comprises conducting a PCR reaction, wherein the number of PCR cycles comprises between about 15 and about 30.
Clause 51. The method of clause 50, wherein the PCR reaction is conducted in a Hydrocycler.
Clause 52. The method of any previous clause, wherein the amplifying results in families of nucleic acid molecules, wherein all nucleic acid molecules in a family share the same combination of barcodes.
Clause 53. The method of any previous clause, wherein the pair of amplification primers is designed to hybridize to the handles at both ends of the tagged nucleic acid molecules and wherein both primers of the pair further comprise an adapter at the 5′ end.
Clause 54. The method of any previous clause, wherein the purifying comprises bead-based extraction, gel extraction, column purification or precipitation.
Clause 55. The method of any previous clause, wherein the barcode primers in the set of barcode primers are between 15 and 50 nucleotides in length.
Clause 56. The method of any previous clause, wherein the number of barcode primers in the set of barcode primers equals the number of barcodes in the first set of nucleic acid tags.
Clause 57. The method of any previous clause, wherein each barcode primer in the set of barcode primers further comprises a label.
Clause 58. The method of clause 57, wherein the label is positioned at the 5′ end of the barcode primer.
Clause 59. The method of anyone of clauses 57 or 58, wherein the label is biotin.
Clause 60. The method of anyone of clauses 57 to 59, wherein the label comprises a sequence region designed to hybridize with a probe.
Clause 61. The method of any previous clause, wherein the set of barcode primers comprises between about 100 and about 10,000 or between about 200 and about 2,000 different barcode-specific regions.
Clause 62. The method of any previous clause, wherein the set of barcode primers comprises a first subset of barcode primers designed to hybridize to barcodes located at the 5′ ends of the nucleic acid molecules and a second subset of barcode primers designed to hybridize to the barcodes located at the 3′ ends of the nucleic acid molecules.
Clause 63. The method of any previous clause, wherein the first and second subset of barcode primers each comprise between about 10 and about 5,000 or between about 100 and about 1,000 different barcode-specific regions.
Clause 64. The method of any previous clause wherein the contacting in step (i) comprises: preloading each second compartment with a different pair of the set of barcode primers and adding at least a portion of the amplified and optionally purified tagged nucleic acid molecules to each second compartment, or preloading each second compartment with at least a portion of the amplified and optionally purified tagged nucleic acid molecules and adding a different pair of the set of barcode primers to each second compartment.
Clause 65. The method of clause 64, wherein each second compartment is provided with (i) the same 5′ barcode primer and a different 3′ barcode primer, (ii) the same 3′ barcode primer and a different 5′ barcode primer, or (iii) a different 5′ primer and a different 3′ primer.
Clause 66. The method of any previous clause wherein the one or more second compartments are selected from wells of a multiwell plate or a cartridge, chambers, vessels, columns or droplets.
Clause 67. The method of anyone of clauses 1 to 65, wherein the one or more second compartments are wells of a 384-well plate or a 1536-well plate, optionally wherein the plates are configured for use with an acoustic liquid handling device.
Clause 68. The method of any previous clause, wherein the identifying in step (j) comprises determining the concentration of nucleic acid molecules in each second compartment, optionally wherein the concentration is determined by qPCR, optical measurement, spectrophotometry or fluorometric detection.
Clause 69. The method of any previous clause, wherein the identifying in step (j) comprises analyzing the amplification products by gel or capillary electrophoresis.
Clause 70. The method of any previous clause, wherein the pooling in step (k) comprises combining at least a portion of the amplification products from multiple second compartments, and optionally contacting the combined amplification products with a further set of nucleic acid tags and attaching the further set of nucleic acid tags to both ends of substantially each amplification product.
Clause 71. The method of any previous clause, wherein the sequencing of the amplification products comprises sequencing by synthesis, sequencing by ligation, Sanger sequencing, single molecule sequencing, third generation sequencing.
Clause 72. The method of clause 71, wherein the sequencing comprises nanopore sequencing or PacBio sequencing.
Clause 73. The method of clause 72, wherein concatemers are formed of the amplification products prior to the sequencing.
Clause 74. The method of any previous clause, wherein the analysing further comprises obtaining the combinations of barcodes flanking the one or more nucleic acid molecules having a desired sequence.
Clause 75. The method of clause 74, wherein the obtaining the combinations of barcodes comprises (a) aligning sequence reads of the one or more nucleic acid molecules having the same barcode combinations and obtaining one or more consensus sequence of the one or more nucleic acid molecules from the aligned sequence reads, (b) comparing the one or more obtained consensus sequence to the desired sequence of the one or more nucleic acid molecules and selecting the sequences of the one or more nucleic acid molecules having a desired sequence, and (c) determining the sequences of the barcodes flanking the desired nucleic acid molecule.
Clause 76. The method of any previous clause, wherein the identifying comprises determining the one or more second compartments comprising the obtained combinations of barcodes.
Clause 77. The method of any previous clause, wherein the method further comprises prior to step (a): subjecting the nucleic acid molecules to an error reduction or error correction step, optionally wherein the error correction or reduction is achieved during or after assembling the nucleic acid molecules, optionally wherein the error correction step comprises one or more heat-stable mismatch cleavage endonuclease, optionally wherein two or more heat-stable mismatch cleavage endonuclease are used with different mismatch specificities.
Clause 78. The method of clause 1 or anyone of clauses 3 to 36 or 38 to 46 or 48 to 77, wherein the method further comprises after step (c) validating the predetermined amount of nucleic acid molecules in the diluted mixtures by qPCR.
Clause 79. The method of any previous clause further comprising: validating a predetermined amount of optionally tagged nucleic acid molecules in one or more diluted mixtures by qPCR.
Clause 80. The method of any previous clause, wherein the method further comprises retrieving one or more amplification products from the identified one or more second compartments.
Clause 81. The method of clause 80, wherein the retrieving comprises pipetting, ejecting or transferring at least a portion of the amplification products from said one or more second compartments.
Clause 82. The method of clause 80, wherein the retrieving comprises hybridizing the amplification products from said one or more second compartments to a probe, optionally wherein said probe is immobilized on a solid support.
Clause 83. The method of any previous clause, wherein the method further comprises prior to step (1): fragmenting the amplification products and/or attaching one or more sequencing adapters to the optionally fragmented amplification products.
Clause 84. The method of any previous clause, wherein the method further comprises combining and assembling two or more of the retrieved amplification products.
Clause 85. The method of clause 84, wherein the assembling comprises restriction enzyme cleavage and ligation, fusion PCR, homologous recombination or exonuclease-based assembly.
Clause 86. The method of clause 84 or 85, wherein prior to the assembling the combined two or more amplification products are cleaved with one or more restriction enzymes at the restriction enzyme cleavage sites of the linkers to remove the tags from the 5′ and 3′ ends and wherein the cleaved amplification products are optionally purified.
Clause 87. the method of clause 86, wherein the one or more restriction enzymes are type IIS restriction endonuclease(s).
Clause 88. The method of anyone of clauses 84 to 87 wherein the assembling results in a synthetic circular or linear nucleic acid molecule, optionally wherein the synthetic linear nucleic acid molecule is covalently closed.
Clause 89. The method of clause 88 further comprising amplifying the synthetic circular or linear nucleic acid molecule wherein optionally the amplifying comprises: rolling circle amplification, strand displacement amplification, or polymerase chain reaction.
Clause 90. The method of any previous clause, wherein the obtained nucleic acid molecule encodes a therapeutic polypeptide or a portion thereof.
Clause 91. The method of clause 88, wherein the synthetic circular or linear nucleic acid molecule is a DNA vector, optionally wherein the DNA vector lacks an origin of replication, a drug resistance gene, CpG motifs, and/or a site-specific recombination recognition site.
Clause 92. Use of a nucleic acid molecule obtained by anyone of the previous clauses as template in an in vitro transcription reaction, in nucleic acid sequencing molecular cloning, medical diagnostics, a nucleic acid vaccine, a self-replicating system, the assembly of a DNA nanostructure, a diagnostic assay, or a biosensor.
Clause 93. An isolated DNA vector comprising one or more nucleic acid molecules obtained by performing a method of anyone of clauses 1-90.

While specific embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### INCORPORATION BY REFERENCE

All publications, patents, and patent applications mentioned in this specification are herein incorporated by reference to the same extent as if each individual publication, patent, or patent application was specifically and individually indicated to be incorporated by reference. This includes the following U.S. Pat. Nos. 8,224,578; 7,164,992; 8,808,989; 4,652,639; 6,083,726; 6,110,668; 7,838,210; 7,833,759; 5,624,827; 6,521,427; 5,869,644; 6,472,184; 6,495,318; 7,704,690; 8,173,368; 7,948,015; 5,580,759; 9,109,250; U.S. Patent Publication Nos. 2007/0141557; 2010/0062495 A1; 2007/0292954 A1; 2003/0152984 AA; 2006/0115850 AA; 2008/0145913; 2006/0127920; 2007/0231805; 2010/0216648; 2011/0124049; 2014/0155297; 2012/0283110; 2012/0322681; 2014/0141982; 2010/0291633 A1; 2008/0287320; 2009/0155858A1; 2012/0053087; 2009/0275086; 2017/0292138; 2018/0087045; 2019/0144489 and PCT Publication Nos. WO 2011/102802; WO 2012/044847; WO 2020/212391; WO 2010/040531; WO 2016/094512; WO 2005/095605 A1; WO 2008/112683; WO 2021/178809; WO 2021/187554; WO 2006/084131; WO 2006/044956 A1 and WO 2020/001783.

1. (canceled)

2. A method for identifying a nucleic acid molecule having a desired sequence in a mixture of nucleic acid molecules comprising the following steps:
(a) providing one or more mixtures of nucleic acid molecules, each mixture comprising a plurality of nucleic acid molecules designed to have a desired sequence;
(b) providing a set of nucleic acid tags, each tag comprising at least
(i) a barcode, and
(ii) a handle or adapter at the 5′ end;
(c) contacting the one or more mixtures of nucleic acid molecules with the set of nucleic acid tags and attaching a tag to both ends of substantially each nucleic acid molecule to obtain tagged nucleic acid molecules having a barcode region and a handle or adapter region at both ends;
(d) providing one or more first compartments and diluting each of the one or more mixtures of tagged nucleic acid molecules in a separate first compartment to obtain diluted mixtures of tagged nucleic acid molecules, each diluted mixture comprising a predetermined amount of tagged nucleic acid molecules;
(e) providing one or more pairs of amplification primers designed to hybridize to both ends of the tagged nucleic acid molecules and amplifying the tagged nucleic acid molecules in the one or more first compartments;
(f) providing a set of barcode primers, each barcode primer of the set comprising a barcode-specific region designed to hybridize to a specific barcode in the set of nucleic acid tags;
(g) providing one or more second compartments and contacting at least a portion of each of the amplified tagged nucleic acid molecules with a defined pair of the set of barcode primers in a second compartment and performing an amplification reaction in each second compartment to obtain one or more second compartments comprising:
(i) one specific amplification product,
(ii) no specific amplification product, and/or
(iii) two or more specific amplification products;
(h) sequencing the amplification products to obtain sequence data;
(i) analysing the sequence data, and
(j) identifying one or more second compartments comprising one specific amplification product, thereby identifying one or more nucleic acid molecules having the desired sequence.

3. (canceled)

4. The method of claim 2, wherein the nucleic acid molecules are linear double-stranded nucleic acid molecules.

5. (canceled)

6. The method of claim 2, wherein the nucleic acid molecules in each mixture have an error rate of less than 1 in 1,000 bp.

7. The method of claim 2, wherein the nucleic acid molecules are between about 200 and about 5,000 nucleotides in length.

8. (canceled)

9. (canceled)

10. (canceled)

11. (canceled)

12. (canceled)

13. (canceled)

14. (canceled)

15. The method of claim 2, wherein each nucleic acid molecule comprises a linker at the 5′ end and the 3′ end and wherein the linker of each nucleic acid molecule further comprises:
(i) a universal primer binding site, and/or
(ii) a restriction enzyme cleavage site.

16. (canceled)

17. (canceled)

18. The method of claim 15, wherein the restriction enzyme cleavage site of the linker is positioned to allow removal of the universal primer binding site from the nucleic acid molecule.

19. (canceled)

20. The method of claim 2, wherein the number of mixtures of nucleic acid molecules is 10 and 500.

21. (canceled)

22. (canceled)

23. (canceled)

24. The method of claim 15, wherein each tag in the set of nucleic acid tags further comprises a linker binding region at the 3′ end, wherein the linker binding region is designed to hybridize to the linker at the 5′ end and/or the 3′ end of each nucleic acid molecule.

25. (canceled)

26. (canceled)

27. The method of claim 2, wherein the set of nucleic acid tags comprises a first subset of tags for attachment to the 5′ end of the nucleic acid molecules and a second subset of tags for attachment to the 3′ end of the nucleic acid molecules and wherein the first and second subset of nucleic acid tags each comprise between 100 and 1,000 different barcodes.

28. (canceled)

29. (canceled)

30. (canceled)

31. (canceled)

32. (canceled)

33. (canceled)

34. (canceled)

35. (canceled)

36. (canceled)

37. (canceled)

38. The method of claim 2, wherein further comprising determining the concentration of nucleic acid molecules in one or more mixtures comprises qPCR, digital PCR, optical measurement, spectrophotometry or fluorometric detection.

39. The method of claim 2, wherein the one or more first or second compartments are selected from wells of a microwell plate, chambers of a cartridge, vessels, or columns.

40. (canceled)

41. (canceled)

42. The method of claim 2, wherein the diluting comprises generating serial dilutions of each mixture of nucleic acid molecules and selecting a dilution determined to comprise a desired number of nucleic acid molecules.

43. (canceled)

44. The method of claim 2, wherein the diluting comprises diluting each mixture of nucleic acid molecules by about 1:10,000 and about 1:10,000,000,000 or generating serial dilutions of between about 10⁻⁴ and about 10⁻¹⁰ of each mixture.

45. (canceled)

46. The method of claim 2, wherein the predetermined amount of nucleic acid molecules in each first compartment represents the number of barcode combinations of the first set of nucleic acid tags.

47. (canceled)

48. (canceled)

49. (canceled)

50. (canceled)

51. (canceled)

52. (canceled)

53. The method of claim 2, wherein the handle comprises a universal primer binding site and wherein the pair of amplification primers is designed to hybridize to the universal primer binding site of the handles at both ends of the tagged nucleic acid molecules and wherein both primers of the pair further comprise an adapter at the 5′ end.

54. (canceled)

55. (canceled)

56. (canceled)

57. (canceled)

58. (canceled)

59. (canceled)

60. (canceled)

61. (canceled)

62. The method of claim 2, wherein the set of barcode primers comprises a first subset of barcode primers designed to hybridize to barcodes located at the 5′ ends of the nucleic acid molecules and a second subset of barcode primers designed to hybridize to the barcodes located at the 3′ ends of the nucleic acid molecules.

63. (canceled)

64. The method of claim 2, wherein the contacting comprises:
preloading each second compartment with a different pair of the set of barcode primers and adding at least a portion of the amplified tagged nucleic acid molecules to each second compartment, or
preloading each second compartment with at least a portion of the amplified tagged nucleic acid molecules and adding a different pair of the set of barcode primers to each second compartment.

65. (canceled)

66. (canceled)

67. (canceled)

68. (canceled)

69. (canceled)

70. (canceled)

71. (canceled)

72. (canceled)

73. (canceled)

74. The method of claim 2, wherein the analysing further comprises obtaining the combinations of barcodes flanking the one or more nucleic acid molecules having a desired sequence comprising:
(a) aligning sequence reads of the one or more nucleic acid molecules having the same barcode combinations and obtaining one or more consensus sequence of the one or more nucleic acid molecules from the aligned sequence reads,
(b) comparing the one or more obtained consensus sequence to the desired sequence of the one or more nucleic acid molecules and selecting the sequences of the one or more nucleic acid molecules having a desired sequence, and
(c) determining the sequences of the barcodes flanking the desired nucleic acid molecule.

75. (canceled)

76. (canceled)

77. (canceled)

78. (canceled)

79. (canceled)

80. The method of claim 2, wherein the identifying comprises determining the one or more second compartments comprising the obtained combination of barcodes and wherein the method further comprises retrieving one or more amplification products from the identified one or more second compartments.

81. (canceled)

82. (canceled)

83. (canceled)

84. The method of claim 80, wherein the method further comprises combining and assembling two or more of the retrieved amplification products.

85. (canceled)

86. (canceled)

87. (canceled)

88. (canceled)

89. (canceled)

90. (canceled)

91. (canceled)

92. (canceled)

93. (canceled)

94. The method of claim 2, further comprising: identifying second compartments (ii) having no specific amplification product and/or second compartments (iii) having two or more specific amplification products and excluding such second compartments from subsequent steps.
